(19) United States
(12) Patent Application Publication
(10) Pub. No.: US 2009/0215801 A9
(43) Pub. Date: Aug. 27, 2009

(54) Novel 2-Aminopyrimidinone Derivatives And Their Use

(75) Inventors: Jeffrey Albert, Wilmington, DE (US); Donald Andisik, Wilmington, DE (US); Phil Edwards, Wilmington, DE (US); Mark Sylvester, Wilmington, DE (US)

Correspondence Address: PEPPER HAMILTON LLP, 400 BERWYN PARK, 899 CASSATT ROAD, BERWYN, PA, 19312-1183, US

(73) Assignee: ASTRAZENECA AB, SODERTAIJE, SWEDENASTEX THEREAPEUTICS LTD, CAMBRIDGE (GB)

(21) Appl. No.: 12/093,670

(22) PCT Filed: Nov. 13, 2006
(86) PCT No.: PCT/SE06/01280

§ 371 Date: Jun. 5, 2008

Related U.S. Application Data
(60) Provisional application No. 60/737,326, filed on Nov. 15, 2005.

(51) Int. Cl. A61K 31/513; C07D 239/22; C07D 239/88; A61K 31/517; C12N 9/99; A61P 25/28

(52) U.S. Cl. 514/266.3; 544/321; 514/272; 544/253; 435/184

(57) ABSTRACT

This invention relates to novel compounds having the structural formula I below: (I) and to their pharmaceutically acceptable salts, compositions and methods of use. These novel compounds provide a treatment or prophylaxis of cognitive impairment, Alzheimer Disease, neurodegeneration and dementia.

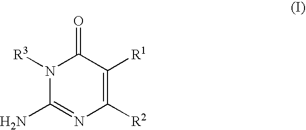

The present invention relates to novel compounds, their pharmaceutical compositions. In addition, the present invention relates to therapeutic methods for the treatment and/or prevention of Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration.

### BACKGROUND OF THE INVENTION

Several groups have identified and isolated aspartate proteinases that have β-secretase activity (Hussain et al., 1999; Lin et. al, 2000; Yan et. al, 1999; Sinha et. al., 1999 and Vassar et. al., 1999). β-secretase is also known in the literature as Asp2 (Yan et. al, 1999), Beta site APP Cleaving Enzyme (BACE) (Vassar et. al., 1999) or memapsin-2 (Lin et al., 2000). BACE was identified using a number of experimental approaches such as EST database analysis (Hussain et al. 1999); expression cloning (Vassar et al. 1999); identification of human homologs from public databases of predicted C. elegans proteins (Yan et al. 1999) and finally utilizing an inhibitor to purify the protein from human brain (Sinha et al. 1999). Thus, five groups employing three different experimental approaches led to the identification of the same enzyme, making a strong case that BACE is a β-secretase. Mention is also made of the patent literature: WO96/40885, EP871720, U.S. Pat. Nos. 5,942,400 and 5,744,346, EP855444, U.S. Pat. No. 6,319,689, WO99/64587, WO99/31236, EP1037977, WO00/17369, WO01/23533, WO0047618, WO00/58479, WO00/69262, WO01/00663, WO01/00665, U.S. Pat. No. 6,313,268.

BACE was found to be a pepsin-like aspartic proteinase, the mature enzyme consisting of the N-terminal catalytic domain, a transmembrane domain, and a small cytoplasmic domain. BACE has an optimum activity at pH 4.0-5.0 (Vassar et al, 1999)) and is inhibited weakly by standard pepsin inhibitors such as pepstatin. It has been shown that the catalytic domain minus the transmembrane and cytoplasmic domain has activity against substrate peptides (Lin et al, 2000). BACE is a membrane bound type 1 protein that is synthesized as a partially active proenzyme, and is abundantly expressed in brain tissue. It is thought to represent the major β-secretase activity, and is considered to be the rate-limiting step in the production of amyloid-β-protein (Aβ). It is thus of special interest in the pathology of Alzheimer's disease, and in the development of drugs as a treatment for Alzheimer's disease.

Aβ or amyloid-β-protein is the major constituent of the brain plaques which are characteristic of Alzheimer's disease (De Strooper et al, 1999). Aβ is a 39-42 residue peptide formed by the specific cleavage of a class I transmembrane protein called APP, or amyloid precursor protein. Aβ-secretase activity cleaves this protein between residues Met671 and Asp672 (numbering of 770aa isoform of APP) to form the N-terminus of Aβ. A second cleavage of the peptide is associated with γ-secretase to form the C-terminus of the Aβ peptide.

Alzheimer's disease (AD) is estimated to afflict more than 20 million people worldwide and is believed to be the most common form of dementia. Alzheimer's disease is a progressive dementia in which massive deposits of aggregated protein breakdown products—amyloid plaques and neurofibrillary tangles accumulate in the brain. The amyloid plaques are thought to be responsible for the mental decline seen in Alzheimer's patients.

The likelihood of developing Alzheimer's disease increases with age, and as the aging population of the developed world increases, this disease becomes a greater and greater problem. In addition to this, there is a familial link to Alzheimer's disease and consequently any individuals possessing the double mutation of APP known as the Swedish mutation (in which the mutated APP forms a considerably improved substrate for BACE) have a much greater chance of developing AD, and also of developing it at an early age (see also U.S. Pat. No. 6,245,964 and U.S. Pat. No. 5,877,399 pertaining to transgenic rodents comprising APP-Swedish). Consequently, there is also a strong need for developing a compound that can be used in a prophylactic fashion for these individuals.

The gene encoding APP is found on chromosome 21, which is also the chromosome found as an extra copy in Down's syndrome. Down's syndrome patients tend to acquire Alzheimer's disease at an early age, with almost all those over 40 years of age showing Alzheimer's-type pathology (Oyama et al., 1994). This is thought to be due to the extra copy of the APP gene found in these patients, which leads to overexpression of APP and therefore to increased levels of APPβ causing the high prevalence of Alzheimer's disease seen in this population. Thus, inhibitors of BACE could be useful in reducing Alzheimer's-type pathology in Down's syndrome patients.

Drugs that reduce or block BACE activity should therefore reduce Aβ levels and levels of fragments of Aβ in the brain, or elsewhere where Aβ or fragments thereof deposit, and thus slow the formation of amyloid plaques and the progression of AD or other maladies involving deposition of Aβ or fragments thereof (Yankner, 1996; De Strooper and Konig, 1999). BACE is therefore an important candidate for the development of drugs as a treatment and/or prophylaxis of Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration.

It would therefore be useful to inhibit the deposition of Aβ and portions thereof by inhibiting BACE through inhibitors such as the compounds provided herein.

The therapeutic potential of inhibiting the deposition of Aβ has motivated many groups to isolate and characterize secretase enzymes and to identify their potential inhibitors (see, e.g., WO01/23533 A2, EP0855444, WO00/17369, WO00/58479, WO00/47618, WO00/77030, WO01/00665, WO01/00663, WO01/29563, WO02/25276, U.S. Pat. No. 5,942,400, U.S. Pat. No. 6,245,884, U.S. Pat. No. 6,221,667, U.S. Pat. No. 6,211,235, WO02/02505, WO02/02506, WO02/02512, WO02/02518, WO02/02520, WO02/14264, WO05/058311, WO 05/097767, US2005/0282826).

The compounds of the present invention show improved properties compared to the potential inhibitors known in the art, e.g. improved hERG selectivity.

### DISCLOSURE OF THE INVENTION

Provided herein are novel compounds of structural formula I:

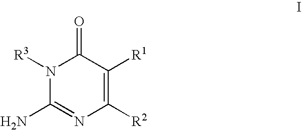

or a pharmaceutically acceptable salt, tautomer or in vivo-hydrolysable precursors thereof, wherein:
R¹ is halo, CN, OR^{a}, SR^{a}, C(O)R^{b}, C(O)NR^{c}R^{d}, C(O)OR^{a}, OC(O)R^{b}, OC(O)NR^{c}R^{d}, S(O)R^{b}, S(O)NR^{c}R^{d}, S(O)₂R^{b}, S(O)₂NR^{c}R^{d}, C₁₋₆ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl, wherein each of the C₁₋₆ alkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl is optionally substituted by 1, 2, 3, 4 or 5 A¹;

### R² is (CR^{2a}R^{2b})₂-Q;

R³ is H, C(O)R^{a}, C(O)OR^{b}, C(O)NR^{c}R^{d}, S(O)R^{a}, S(O)₂R^{a}, C₁₋₁₀ alkyl, C₂₋₁₀ alkenyl, C₂₋₁₀ alkynyl, cycloalkyl, heterocycloalkyl, arylalkyl, heteroarylalkyl, cycloalkylalkyl or heterocycloalkylalkyl, wherein the C₁₋₁₀ alkyl, C₂₋₁₀ alkenyl, C₂₋₁₀ alkynyl, cycloalkyl, heterocycloalkyl, arylalkyl, heteroarylalkyl, cycloalkylalkyl or heterocycloalkylalkyl are each optionally substituted with 1, 2, 3, 4 or 5 A²;
R^{2a} and R^{2b} are each, independently, H, halo, C₁₋₄ alkyl, C₁₋₄ haloalkyl, C₂₋₆ alkenyl, C₂₋₆ alkynyl, aryl, cycloalkyl, heteroaryl, heterocycloalkyl, CN, NO₂, OR^{a′}, SR^{a′}, C(O)R^{b′}, C(O)NR^{c′}R^{d′}, C(O)OR^{a′}, OC(O)R^{b′}, OC(O)NR^{c′}R^{d′}, NR^{c′}R^{d′}, NR^{c′}C(O)R^{d′}, NR^{c′}C(O)OR^{a′}, NR^{c′}S(O)₂R^{b′}, S(O)R^{b′}, S(O)NR^{c′}R^{d′}, S(O)₂R^{b′}, or S(O)₂NR^{c′}R^{d′};
Q is aryl, cycloalkyl, heteroaryl or heterocycloalkyl, each optionally substituted by 1, 2, 3, 4 or 5 Cy¹ or R^{Q};
Cy¹ is aryl, heteroaryl, cycloalkyl, or heterocycloalkyl, each optionally substituted with 1, 2, 3, 4 or 5 A³;
A¹, A², and A³ are each, independently, halo, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, NR^{c}S(O)R^{b}, NR^{c}S(O)₂R^{b}, S(O)R^{b}, S(O)NR^{c}R^{d}, S(O)₂R^{b}, S(O)₂NR^{c}R^{d}, C₁₋₄ alkoxy, C₁₋₄haloalkoxy, amino, C₁₋₄ alkylamino, C₂₋₈ dialkylamino, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl or heterocycloalkyl, wherein each of the C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl or heterocycloalkyl is optionally substituted by 1, 2, 3, 4 or 5 halo, C₁₋₆ alkyl, C₂₋₆ alkenyl, C₂₋₆ alkynyl, C₁₋₄ haloalkyl, aryl, cycloalkyl, heteroaryl, heterocycloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, NR^{c}S(O)R^{b}, NR^{c}S(O)₂R^{b}, S(O)R^{b}, S(O)NR^{c}R^{d}, S(O)₂R^{b}, or S(O)₂NR^{c}R^{d};
R^{Q} is halo, CN, NO₂, OR^{a}, SR^{a}, C(O)R^{b}, C(O)NR^{c}R^{d}, C(O)OR^{a}, OC(O)R^{b}, OC(O)NR^{c}R^{d}, S(O)R^{b}, S(O)NR^{c}R^{d}, S(O)₂R^{b}, S(O)₂NR^{c}R^{d}, C₁₋₄ alkoxy, C₁₋₄ haloalkoxy, C₁₋₆ alkyl, C₂₋₆ alkenyl, C₂₋₆ alkynyl, arylalkyl, cycloalkylalkyl, heteroarylalkyl or heterocycloalkylalkyl, wherein each of the C₁₋₆ alkyl, C₂₋₆ alkenyl, C₂₋₆ alkynyl, arylalkyl, cycloalkylalkyl, heteroarylalkyl or heterocycloalkylalkyl is optionally substituted by 1, 2, 3, 4 or 5 halo, C₁₋₆ alkyl, C₂₋₆ alkenyl, C₂₋₆ alkynyl, C₁₋₄ haloalkyl, aryl, cycloalkyl, heteroaryl, heterocycloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, NR^{c}S(O)R^{b}, NR^{c}S(O)₂R^{b}, S(O)R^{b}, S(O)NR^{c}R^{d}, S(O)₂R^{b}, or S(O)₂NR^{c}R^{d};
R^{a} and R^{a′} are each, independently, H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl, wherein the C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl is optionally substituted with OH, amino, halo, C₁₋₆ alkyl, C₁₋₆ haloalkyl, aryl, arylalkyl, heteroaryl, heteroarylalkyl, cycloalkyl or heterocycloalkyl;
R^{b} and R^{b′} are each, independently, H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl, wherein the C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl is optionally substituted with OH, amino, halo, C₁₋₆ alkyl, C₁₋₆ haloalkyl, C₁₋₆haloalkyl, aryl, arylalkyl, heteroaryl, heteroarylalkyl, cycloalkyl or heterocycloalkyl;
R^{c} and R^{d} are each, independently, H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl, wherein the C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl is optionally substituted with OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;
or R^{c} and R^{d} together with the N atom to which they are attached form a 4-, 5-, 6- or 7-membered heterocycloalkyl group; and
R^{c′} and R^{d′} are each, independently, H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl, wherein the C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl is optionally substituted with OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;
or R^{c′} and R^{d′} together with the N atom to which they are attached form a 4-, 5-, 6- or 7-membered heterocycloalkyl group.

In some embodiments, R¹ is halo, C₁₋₆ alkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl, wherein each of the C₁₋₆ alkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl is optionally substituted by 1, 2 or 3 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl.

In some embodiments, R¹ is halo, C₁₋₆ alkyl, aryl, heteroaryl, cycloalkyl, heterocycloalkyl, arylalkyl, heteroarylalkyl, cycloalkylalkyl or heterocycloalkylalkyl.

In some embodiments, R^{2a} and R^{2b} are each, independently, H, halo, C₁₋₄ alkyl, C₁₋₄ haloalkyl, C₂₋₆ alkenyl, C₂₋₆ alkynyl, aryl, cycloalkyl, heteroaryl, heterocycloalkyl, NO₂, OR^{a′}, SR^{a′}, OC(O)R^{b′}, OC(O)NR^{c′}R^{d′}, S(O)R^{b′}, S(O)NR^{c′}R^{d′}, S(O)₂R^{b′}, or S(O)₂NR^{c′}R^{d′}.

In some embodiments, R^{2a} and R^{2b} are each, independently, H, halo, C₁₋₄ alkyl, C₁₋₄ haloalkyl, C₂₋₆ alkenyl, C₂₋₆ alkynyl, aryl, cycloalkyl, heteroaryl or heterocycloalkyl.

In some embodiments, R^{2a} and R^{2b} are each, independently, H or C₁₋₄ alkyl.

In some embodiments, R^{2a} and R^{2b} are both H.

In some embodiments, Q is aryl or heteroaryl, each optionally substituted by 1, 2, 3, 4 or 5 Cy¹ or R^{Q}.

In some embodiments, Q is aryl optionally substituted by 1, 2, 3, 4 or 5 Cy¹ or R^{Q}.

In some embodiments, Q is aryl optionally substituted by 1, 2 or 3 R^{Q}.

In some embodiments, Q is aryl substituted by Cy¹ and optionally substituted by 1, 2 or 3 R^{Q}.

In some embodiments, Q is aryl substituted by Cy¹ and optionally substituted by 1, 2 or 3 R^{Q}; and Cy¹ is aryl or heteroaryl, each optionally substituted by 1, 2, 3, 4 or 5 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl.

In some embodiments, Q is phenyl wherein the phenyl is meta-substituted by Cy¹; and Cy¹ is aryl or heteroaryl, each optionally substituted by 1, 2, 3, 4 or 5 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl.

In some embodiments, Q is phenyl wherein the phenyl is meta-substituted by Cy¹; and Cy¹ is aryl optionally substituted by 1, 2, 3, 4 or 5 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl.

In some embodiments, R³ is C₁₋₁₀ alkyl, C₂₋₁₀ alkenyl, C₂₋₁₀ alkynyl, cycloalkyl, heterocycloalkyl, arylalkyl, heteroarylalkyl, cycloalkylalkyl or heterocycloalkylalkyl, wherein the C₁₋₁₀ alkyl, C₂₋₁₀ alkenyl, C₂₋₁₀ alkynyl, cycloalkyl, heterocycloalkyl, arylalkyl, heteroarylalkyl, cycloalkylalkyl or heterocycloalkylalkyl are each optionally substituted with 1, 2, 3, 4 or 5 A².

In some embodiments, R³ is C₁₋₁₀ alkyl, arylalkyl, heteroarylalkyl, cycloalkylalkyl or heterocycloalkylalkyl, wherein the C₁₋₁₀ alkyl, arylalkyl, heteroarylalkyl, cycloalkylalkyl or heterocycloalkylalkyl are each optionally substituted with 1, 2, 3, 4 or 5 A².

In some embodiments, R³ is C₁₋₁₀ alkyl, arylalkyl, heteroarylalkyl, cycloalkylalkyl or heterocycloalkylalkyl, wherein the C₁₋₁₀ alkyl, arylalkyl, heteroarylalkyl, cycloalkylalkyl or heterocycloalkylalkyl are each optionally substituted with 1, 2 or 3 A²; and A² is halo, CN, NO₂, OR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, NR^{c}S(O)₂R^{b}, S(O)₂R^{b}, S(O)₂NR^{c}R^{d}, C₁₋₄ alkoxy, C₁₋₄ haloalkoxy, amino, C₁₋₄ alkylamino, C₂₋₈ dialkylamino, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl or heterocycloalkyl, wherein each of the C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl or heterocycloalkyl is optionally substituted by 1, 2, 3, 4 or 5 halo, C₁₋₆ alkyl, C₂₋₆ alkenyl, C₂₋₆ alkynyl, C₁₋₄ haloalkyl, aryl, cycloalkyl, heteroaryl, heterocycloalkyl, CN, NO₂, OR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, NR^{c}S(O)₂R^{b}, S(O)R^{b}, S(O)₂R^{b}, or S(O)₂NR^{c}R^{d}.

In some embodiments, R³ is C₁₋₁₀ alkyl, arylalkyl, heteroarylalkyl, cycloalkylalkyl or heterocycloalkylalkyl.

In some embodiments, R³ is C₁₋₁₀ alkyl.

In some embodiments, R¹ is halo, C₁₋₆ alkyl, aryl, heteroaryl, cycloalkyl, heterocycloalkyl, arylalkyl, heteroarylalkyl, cycloalkylalkyl or heterocycloalkylalkyl; R^{2a} and R^{2b} are each, independently, H or C₁₋₄ alkyl; Q is aryl optionally substituted by 1, 2 or 3 R^{Q}; and R³ is C₁₋₁₀ alkyl, arylalkyl, heteroarylalkyl, cycloalkylalkyl or heterocycloalkylalkyl, wherein the C₁₋₁₀ alkyl, arylalkyl, heteroarylalkyl, cycloalkylalkyl or heterocycloalkylalkyl are each optionally substituted with 1, 2, 3, 4 or 5 A².

In some embodiments, R¹ is halo, C₁₋₆ alkyl, aryl, heteroaryl, cycloalkyl, heterocycloalkyl, arylalkyl, heteroarylalkyl, cycloalkylalkyl or heterocycloalkylalkyl; R^{2a} and R^{2b} are each, independently, H or C₁₋₄ alkyl; Q is phenyl optionally substituted by 1, 2 or 3 halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, or heterocycloalkylalkyl; and R³ is C₁₋₁₀ alkyl, arylalkyl, heteroarylalkyl, cycloalkylalkyl or heterocycloalkylalkyl, wherein the C₁₋₁₀ alkyl, arylalkyl, heteroarylalkyl, cycloalkylalkyl or heterocycloalkylalkyl are each optionally substituted with 1, 2, 3, 4 or 5 halo, CN, NO₂, OR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, NR^{c}S(O)₂R^{b}, S(O)₂R^{b}, S(O)₂NR^{c}R^{d}, C₁₋₄ alkoxy, C₁₋₄ haloalkoxy, amino, C₁₋₄ alkylamino, C₂₋₈ dialkylamino, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl or heterocycloalkyl.

In some embodiments, Q is phenyl meta-substituted by halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, or heterocycloalkylalkyl.

Also provided herein are novel compounds of structural formula II:

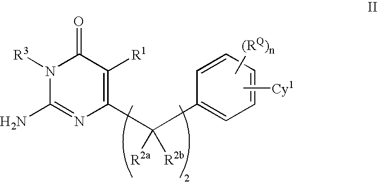

wherein:
R¹ is halo, C₁₋₆ alkyl, aryl, heteroaryl, cycloalkyl, heterocycloalkyl, arylalkyl, heteroarylalkyl, cycloalkylalkyl or heterocycloalkylalkyl;
R^{2a} and R^{2b} are each, independently, H or C₁₋₄ alkyl;
R³ is C₁₋₁₀ alkyl, arylalkyl, heteroarylalkyl, cycloalkylalkyl or heterocycloalkylalkyl, wherein the C₁₋₁₀ alkyl, arylalkyl, heteroarylalkyl, cycloalkylalkyl or heterocycloalkylalkyl are each optionally substituted with 1, 2, 3, 4 or 5 halo, CN, NO₂, OR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, NR^{c}S(O)₂R^{b}, S(O)₂R^{b}, S(O)₂NR^{c}R^{d}, C₁₋₄ alkoxy, C₁₋₄ haloalkoxy, amino, C₁₋₄ alkylamino, C₂₋₈ dialkylamino, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl or heterocycloalkyl;
R^{Q} is halo, CN, C₁₋₄ alkoxy, C₁₋₄ haloalkoxy, C₁₋₆ alkyl, C₂₋₆ alkenyl, C₂₋₆ alkynyl, arylalkyl, cycloalkylalkyl, heteroarylalkyl or heterocycloalkylalkyl;
Cy¹ is aryl or heteroaryl, each optionally substituted by 1, 2, 3, 4 or 5 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl; and
n is 0 or 1.

Also provided herein are novel compounds of structural formula III:

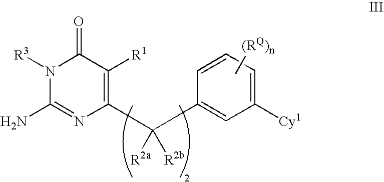

wherein:
R¹ is halo, C₁₋₆ alkyl, aryl, heteroaryl, cycloalkyl, heterocycloalkyl, arylalkyl, heteroarylalkyl, cycloalkylalkyl or heterocycloalkylalkyl;
R^{2a} and R^{2b} are each, independently, H or C₁₋₄ alkyl;
R³ is C₁₋₁₀ alkyl, arylalkyl, heteroarylalkyl, cycloalkylalkyl or heterocycloalkylalkyl, wherein the C₁₋₁₀ alkyl, arylalkyl, heteroarylalkyl, cycloalkylalkyl or heterocycloalkylalkyl are each optionally substituted with 1, 2, 3, 4 or 5 halo, CN, NO₂, OR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, NR^{c}S(O)₂R^{b}, S(O)₂R^{b}, S(O)₂NR^{c}R^{d}, C₁₋₄ alkoxy, C₁₋₄haloalkoxy, amino, C₁₋₄ alkylamino, C₂₋₈ dialkylamino, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl or heterocycloalkyl;
R^{Q} is halo, CN, C₁₋₄ alkoxy, C₁₋₄ haloalkoxy, C₁₋₆ alkyl, C₂₋₆ alkenyl, C₂₋₆ alkynyl, arylalkyl, cycloalkylalkyl, heteroarylalkyl or heterocycloalkylalkyl;
Cy¹ is aryl or heteroaryl, each optionally substituted by 1, 2, 3, 4 or 5 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl; and
n is 0 or 1.

In some embodiments, n is 0.

In some embodiments, n is 0; Cy¹ is phenyl optionally substituted by 1, 2, 3, 4 or 5 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl.

Also provided herein are novel compounds of structural formula IV:

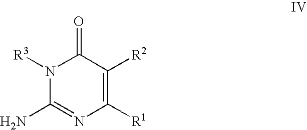

or a pharmaceutically acceptable salt, tautomer or in vivo-hydrolysable precursors thereof,
wherein:
R¹ is H, halo, CN, OR^{a}, SR^{a}, C(O)R^{b}, C(O)NR^{c}R^{d}, C(O)OR^{a}, OC(O)R^{b}, OC(O)NR^{c}R^{d}, S(O)R^{b}, S(O)NR^{c}R^{d}, S(O)₂R^{b}, S(O)₂NR^{c}R^{d}, C₁₋₆ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl, wherein each of the C₁₋₆ alkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl is optionally substituted by 1, 2, 3, 4 or 5 A¹;

### R² is (CR^{2a}R^{2b})₂-Q;

R³ is C(O)R^{a}, C(O)OR^{b}, C(O)NR^{c}R^{d}, S(O)R^{a}, S(O)₂R^{a}, C₁₋₁₀ alkyl, C₂₋₁₀ alkenyl, C₂₋₁₀ alkynyl, cycloalkyl, heterocycloalkyl, arylalkyl, heteroarylalkyl, cycloalkylalkyl or heterocycloalkylalkyl, wherein the C₁₋₁₀ alkyl, C₂₋₁₀ alkenyl, C₂₋₁₀ alkynyl, cycloalkyl, heterocycloalkyl, arylalkyl, heteroarylalkyl, cycloalkylalkyl or heterocycloalkylalkyl are each optionally substituted with 1, 2, 3, 4 or 5 A²;
R^{2a} and R^{2b} are each, independently, H, halo, C₁₋₄ alkyl, C₁₋₄ haloalkyl, C₂₋₆ alkenyl, C₂₋₆ alkynyl, aryl, cycloalkyl, heteroaryl, heterocycloalkyl, CN, NO₂, OR^{a′}, SR^{a′}, C(O)R^{b′}, C(O)NR^{c′}R^{d′}, C(O)OR^{a′}, OC(O)R^{b′}, OC(O)NR^{c′}R^{d′}, NR^{c′}R^{d′}, NR^{c′}C(O)R^{d′}, NR^{c′}C(O)OR^{a′}, NR^{c′}S(O)₂R^{b′}, S(O)R^{b′}, S(O)NR^{c′}R^{d′}, S(O)₂R^{b′}, or S(O)₂NR^{c′}R^{d′};
Q is aryl, heteroaryl or cycloalkyl, each optionally substituted by 1, 2, 3, 4 or 5 Cy¹ or R^{Q};
Cy¹ is aryl, heteroaryl or cycloalkyl, each optionally substituted with 1, 2, 3, 4 or 5 A³;
A¹, A², and A³ are each, independently, halo, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, NR^{c}S(O)R^{b}; NR^{c}S(O)₂R^{b}, S(O)R^{b}, S(O)NR^{c}R^{d}, S(O)₂R^{b}, S(O)₂NR^{c}R^{d}, C₁₋₄ alkoxy, C₁₋₄ haloalkoxy, amino, C₁₋₄ alkylamino, C₂₋₈ dialkylamino, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl or heterocycloalkyl, wherein each of the C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl or heterocycloalkyl is optionally substituted by 1, 2, 3, 4 or 5 halo, C₁₋₆ alkyl, C₂₋₆ alkenyl, C₂₋₆ alkynyl, C₁₋₄ haloalkyl, aryl, cycloalkyl, heteroaryl, heterocycloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, NR^{c}S(O)R^{b}, NR^{c}S(O)₂R^{b}, S(O)R^{b}, S(O)NR^{c}R^{d}, S(O)₂R^{b}, or S(O)₂NR^{c}R^{d};
R^{Q} is halo, CN, NO₂, OR^{a}, SR^{a}, C(O)R^{b}, C(O)NR^{c}R^{d}, C(O)OR^{a}, OC(O)R^{b}, OC(O)NR^{c}R^{d}, S(O)R^{b}, S(O)NR^{c}R^{d}, S(O)₂R^{b}, S(O)₂NR^{c}R^{d}, C₁₋₄ alkoxy, C₁₋₄haloalkoxy, C₁₋₆ alkyl, C₂₋₆ alkenyl, C₂₋₆ alkynyl, arylalkyl, cycloalkylalkyl, heteroarylalkyl or heterocycloalkylalkyl, wherein each of the C₁₋₆ alkyl, C₂₋₆ alkenyl, C₂₋₆ alkynyl, arylalkyl, cycloalkylalkyl, heteroarylalkyl or heterocycloalkylalkyl is optionally substituted by 1, 2, 3, 4 or 5 halo, C₁₋₆ alkyl, C₂₋₆ alkenyl, C₂₋₆ alkynyl, C₁₋₄ haloalkyl, aryl, cycloalkyl, heteroaryl, heterocycloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, NR^{c}S(O)R^{b}, NR^{c}S(O)₂R^{b}, S(O)R^{b}, S(O)NR^{c}R^{d}, S(O)₂R^{b}, or S(O)₂NR^{c}R^{d};
R^{a} and R^{a′} are each, independently, H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl, wherein the C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl is optionally substituted with OH, amino, halo, C₁₋₆ alkyl, C₁₋₆ haloalkyl, aryl, arylalkyl, heteroaryl, heteroarylalkyl, cycloalkyl or heterocycloalkyl;
R^{b} and R^{b′} are each, independently, H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl, wherein the C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl is optionally substituted with OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;
R^{c} and R^{d} are each, independently, H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl, wherein the C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl is optionally substituted with OH, amino, halo, C₁₋₆ alkyl, C₁₋₆ haloalkyl, C₁₋₆haloalkyl, aryl, arylalkyl, heteroaryl, heteroarylalkyl, cycloalkyl or heterocycloalkyl;
or R^{c} and R^{d} together with the N atom to which they are attached form a 4-, 5-, 6- or 7-membered heterocycloalkyl group; and
R^{c′} and R^{d′} are each, independently, H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl, wherein the C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl is optionally substituted with OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;
or R^{c′} and R^{d′} together with the N atom to which they are attached form a 4-, 5-, 6- or 7-membered heterocycloalkyl group.

In some embodiments, R¹ is H, C₁₋₆ alkyl, aryl, heteroaryl, cycloalkyl, heterocycloalkyl, arylalkyl, heteroarylalkyl, cycloalkylalkyl or heterocycloalkylalkyl.

In some embodiments, R^{2a} and R^{2b} are each, independently, H, halo, C₁₋₄ alkyl, C₁₋₄ haloalkyl, C₂₋₆ alkenyl, C₂₋₆ alkynyl, aryl, cycloalkyl, heteroaryl, heterocycloalkyl, NO₂, OR^{a′}, SR^{a′}, OC(O)R^{b′}, OC(O)NR^{c′}R^{d′}, S(O)R^{b′}, S(O)NR^{c′}R^{d′}, S(O)₂R^{b′}, or S(O)₂NR^{c′}R^{d′}.

In some embodiments, R^{2a} and R^{2b} are each, independently, H, halo, C₁₋₄ alkyl, C₁₋₄ haloalkyl, C₂₋₆ alkenyl, C₂₋₆ alkynyl, aryl, cycloalkyl, heteroaryl or heterocycloalkyl.

In some embodiments, R^{2a} and R^{2b} are each, independently, H or C₁₋₄ alkyl.

In some embodiments, R^{2a} and R^{2b} are both H.

In some embodiments, Q is aryl or heteroaryl, each optionally substituted by 1, 2, 3, 4 or 5 Cy¹ or R^{Q}.

In some embodiments, Q is aryl optionally substituted by 1, 2, 3, 4 or 5 Cy¹ or R^{Q}.

In some embodiments, Q is aryl optionally substituted by 1, 2 or 3 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl or heterocycloalkylalkyl.

In some embodiments, Q is aryl substituted by Cy¹ and optionally substituted by 1, 2 or 3 R^{Q}.

In some embodiments, Q is aryl substituted by Cy¹ and optionally substituted by 1, 2 or 3 R^{Q}; Cy¹ is aryl or heteroaryl, each optionally substituted by 1, 2, 3, 4 or 5 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl.

In some embodiments, Q is phenyl wherein the phenyl is meta-substituted by Cy¹; and Cy¹ is aryl or heteroaryl, each optionally substituted by 1, 2, 3, 4 or 5 substituents independently selected from halo, CN, OH, C₁₋₆ alkoxy, C₁₋₆haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl.

In some embodiments, Q is phenyl wherein the phenyl is meta-substituted by Cy¹; and Cy¹ is aryl optionally substituted by 1, 2, 3, 4 or 5 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl.

In some embodiments, R³ is C₁₋₁₀ alkyl, C₂₋₁₀ alkenyl, C₂₋₁₀ alkynyl, cycloalkyl, heterocycloalkyl, arylalkyl, heteroarylalkyl, cycloalkylalkyl or heterocycloalkylalkyl, wherein the C₁₋₁₀ alkyl, C₂₋₁₀ alkenyl, C₂₋₁₀ alkynyl, cycloalkyl, heterocycloalkyl, arylalkyl, heteroarylalkyl, cycloalkylalkyl or heterocycloalkylalkyl are each optionally substituted with 1, 2, 3, 4 or 5 A².

In some embodiments, R³ is C₁₋₁₀ alkyl, arylalkyl, heteroarylalkyl, cycloalkylalkyl or heterocycloalkylalkyl, wherein the C₁₋₁₀ alkyl, arylalkyl, heteroarylalkyl, cycloalkylalkyl or heterocycloalkylalkyl are each optionally substituted with 1, 2, 3, 4 or 5 A².

In some embodiments, R³ is C₁₋₁₀ alkyl, arylalkyl, heteroarylalkyl, cycloalkylalkyl or heterocycloalkylalkyl, wherein the C₁₋₁₀ alkyl, arylalkyl, heteroarylalkyl, cycloalkylalkyl or heterocycloalkylalkyl are each optionally substituted with 1, 2 or 3 A²; and A² is halo, CN, NO₂, OR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, NR^{c}S(O)₂R^{b}, S(O)₂R^{b}, S(O)₂NR^{c}R^{d}, C₁₋₄ alkoxy, C₁₋₄haloalkoxy, amino, C₁₋₄ alkylamino, C₂₋₈ dialkylamino, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl or heterocycloalkyl, wherein each of the C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl or heterocycloalkyl is optionally substituted by 1, 2, 3, 4 or 5 halo, C₁₋₆ alkyl, C₂₋₆ alkenyl, C₂₋₆ alkynyl, C₁₋₄ haloalkyl, aryl, cycloalkyl, heteroaryl, heterocycloalkyl, CN, NO₂, OR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, NR^{c}S(O)₂R^{b}, S(O)R^{b}, S(O)₂R^{b}, or S(O)₂NR^{c}R^{d}.

In some embodiments, R³ is C₁₋₁₀ alkyl, arylalkyl, heteroarylalkyl, cycloalkylalkyl or heterocycloalkylalkyl.

In some embodiments, R³ is C₁₋₁₀ alkyl.

Also provided herein are novel compounds of structural formula V:

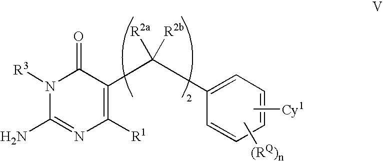

wherein:
R¹ is H, C₁₋₆ alkyl, aryl, heteroaryl, cycloalkyl, heterocycloalkyl, arylalkyl, heteroarylalkyl, cycloalkylalkyl or heterocycloalkylalkyl;
R^{2a} and R^{2b} are each, independently, H or C₁₋₄ alkyl;
R³ is C₁₋₁₀ alkyl, arylalkyl, heteroarylalkyl, cycloalkylalkyl or heterocycloalkylalkyl, wherein the C₁₋₁₀ alkyl, arylalkyl, heteroarylalkyl, cycloalkylalkyl or heterocycloalkylalkyl are each optionally substituted with 1, 2, 3, 4 or 5 halo, CN, NO₂, OR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, NR^{c}S(O)₂R^{b}, S(O)₂R^{b}, S(O)₂NR^{c}R^{d}, C₁₋₄ alkoxy, C₁₋₄ haloalkoxy, amino, C₁₋₄ alkylamino, C₂₋₈ dialkylamino, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl or heterocycloalkyl;
R^{Q} is halo, CN, C₁₋₄ alkoxy, C₁₋₄ haloalkoxy, C₁₋₆ alkyl, C₂₋₆ alkenyl, C₂₋₆ alkynyl, arylalkyl, cycloalkylalkyl, heteroarylalkyl or heterocycloalkylalkyl;
Cy¹ is aryl or heteroaryl, each optionally substituted by 1, 2, 3, 4 or 5 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl; and
n is 0 or 1.

Also provided herein are novel compounds of structural formula VI:

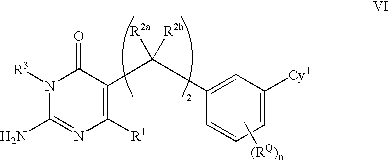

wherein:
R¹ is H, C₁₋₆ alkyl, aryl, heteroaryl, cycloalkyl, heterocycloalkyl, arylalkyl, heteroarylalkyl, cycloalkylalkyl or heterocycloalkylalkyl;
R^{2a} and R^{2b} are each, independently, H or C₁₋₄ alkyl;
R³ is C₁₋₁₀ alkyl, arylalkyl, heteroarylalkyl, cycloalkylalkyl or heterocycloalkylalkyl, wherein the C₁₋₁₀ alkyl, arylalkyl, heteroarylalkyl, cycloalkylalkyl or heterocycloalkylalkyl are each optionally substituted with 1, 2, 3, 4 or 5 halo, CN, NO₂, OR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, NR^{c}S(O)₂R^{b}, S(O)₂R^{b}, S(O)₂NR^{c}R^{d}, C₁₋₄ alkoxy, C₁₋₄haloalkoxy, amino, C₁₋₄ alkylamino, C₂₋₈ dialkylamino, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl or heterocycloalkyl;
R^{Q} is halo, CN, C₁₋₄ alkoxy, C₁₋₄ haloalkoxy, C₁₋₆ alkyl, C₂₋₆ alkenyl, C₂₋₆ alkynyl, arylalkyl, cycloalkylalkyl, heteroarylalkyl or heterocycloalkylalkyl;
Cy¹ is aryl or heteroaryl, each optionally substituted by 1, 2, 3, 4 or 5 substituents independently selected from halo, CN, OH, C₁₋₆ alkoxy, C₁₋₆haloalkoxy, C₁₋₆haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl; and
n is 0 or 1.

In some embodiments, n is 0.

In some embodiments, n is 0; and Cy¹ is phenyl optionally substituted by 1, 2, 3, 4 or 5 substituents independently selected from halo, CN, OH, C₁₋₆ alkoxy, C₁₋₆haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl.

Also provided herein are novel compounds of structural formula VII:

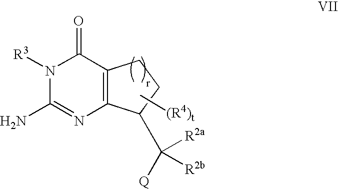

or a pharmaceutically acceptable salt, tautomer or in vivo-hydrolysable precursors thereof, wherein:
R³ is H, C(O)R^{a}, C(O)OR^{b}, C(O)NR^{c}R^{d}, S(O)R^{a}, S(O)₂R^{a}, C₁₋₁₀ alkyl, C₂₋₁₀ alkenyl, C₂₋₁₀ alkynyl, cycloalkyl, heterocycloalkyl, arylalkyl, heteroarylalkyl, cycloalkylalkyl or heterocycloalkylalkyl, wherein the C₁₋₁₀ alkyl, C₂₋₁₀ alkenyl, C₂₋₁₀ alkynyl, cycloalkyl, heterocycloalkyl, arylalkyl, heteroarylalkyl, cycloalkylalkyl or heterocycloalkylalkyl are each optionally substituted with 1, 2, 3, 4 or 5 A²;
R⁴ is halo, CN, OR^{a}, SR^{a}, C(O)R^{b}, C(O)NR^{c}R^{d}, C(O)OR^{a}, OC(O)R^{b}, OC(O)NR^{c}R^{d}, S(O)R^{b}, S(O)NR^{c}R^{d}, S(O)₂R^{b}, S(O)₂NR^{c}R^{d}, C₁₋₆ alkyl, C₁₋₆ haloalkyl, aryl, heteroaryl, cycloalkyl, heterocycloalkyl, arylalkyl, heteroarylalkyl, cycloalkylalkyl or heterocycloalkylalkyl, wherein the C₁₋₆ alkyl, aryl, heteroaryl, cycloalkyl, heterocycloalkyl, arylalkyl, heteroarylalkyl, cycloalkylalkyl or heterocycloalkylalkyl is optionally substituted by 1, 2, 3, 4 or 5 A¹;
R^{2a} and R^{2b} are each, independently, H, halo, C₁₋₄ alkyl, C₁₋₄ haloalkyl, C₂₋₆ alkenyl, C₂₋₆ alkynyl, aryl, cycloalkyl, heteroaryl, heterocycloalkyl, CN, NO₂, OR^{a′}, SR^{a′}, C(O)R^{b′}, C(O)NR^{c′}R^{d′}, C(O)OR^{a′}, OC(O)R^{b′}, OC(O)NR^{c′}R^{d′}, NR^{c′}R^{d′}, NR^{c′}C(O)R^{d′}, NR^{c′}C(O)OR^{a′}, NR^{c′}S(O)₂R^{b′}, S(O)R^{b′}, S(O)NR^{c′}R^{d′}, S(O)₂R^{b′}, or S(O)₂NR^{c′}R^{d′};
r is 0, 1, 2 or 3;
t is 0, 1, 2, 3, 4 or 5;
Q is aryl, cycloalkyl, heteroaryl or heterocycloalkyl, each optionally substituted by 1, 2, 3, 4 or 5 Cy¹ or R^{Q};
Cy¹ is aryl, heteroaryl, cycloalkyl, or heterocycloalkyl, each optionally substituted with 1, 2, 3, 4 or 5 A³;
A¹, A², and A³ are each, independently, halo, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, NR^{c}S(O)R^{b}, NR^{c}S(O)₂R^{b}, S(O)R^{b}, S(O)NR^{c}R^{d}, S(O)₂R^{b}, S(O)₂NR^{c}R^{d}, C₁₋₄ alkoxy, C₁₋₄haloalkoxy, amino, C₁₋₄ alkylamino, C₂₋₈ dialkylamino, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl or heterocycloalkyl, wherein each of the C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl or heterocycloalkyl is optionally substituted by 1, 2, 3, 4 or 5 halo, C₁₋₆ alkyl, C₂₋₆ alkenyl, C₂₋₆ alkynyl, C₁₋₄ haloalkyl, aryl, cycloalkyl, heteroaryl, heterocycloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, NR^{c}S(O)R^{b}, NR^{c}S(O)₂R^{b}, S(O)R^{b}, S(O)NR^{c}R^{d}, S(O)₂R^{b}, or S(O)₂NR^{c}R^{d};
R^{Q} is halo, CN, NO₂, OR^{a}, SR^{a}, C(O)R^{b}, C(O)NR^{c}R^{d}, C(O)OR^{a}, OC(O)R^{b}, OC(O)NR^{c}R^{d}, S(O)R^{b}, S(O)NR^{c}R^{d}, S(O)₂R^{b}, S(O)₂NR^{c}R^{d}, C₁₋₄ alkoxy, C₁₋₄ haloalkoxy, C₁₋₆ alkyl, C₂₋₆ alkenyl, C₂₋₆ alkynyl, arylalkyl, cycloalkylalkyl, heteroarylalkyl or heterocycloalkylalkyl, wherein each of the C₁₋₆ alkyl, C₂₋₆ alkenyl, C₂₋₆ alkynyl, arylalkyl, cycloalkylalkyl, heteroarylalkyl or heterocycloalkylalkyl is optionally substituted by 1, 2, 3, 4 or 5 halo, C₁₋₆ alkyl, C₂₋₆ alkenyl, C₂₋₆ alkynyl, C₁₋₄ haloalkyl, aryl, cycloalkyl, heteroaryl, heterocycloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, NR^{c}S(O)R^{b}, NR^{c}S(O)₂R^{b}, S(O)R^{b}, S(O)NR^{c}R^{d}, S(O)₂R^{b}, or S(O)₂NR^{c}R^{d};
R^{a} and R^{a′} are each, independently, H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl, wherein the C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl is optionally substituted with OH, amino, halo, C₁₋₆ alkyl, C₁₋₆ haloalkyl, aryl, arylalkyl, heteroaryl, heteroarylalkyl, cycloalkyl or heterocycloalkyl;
R^{b} and R^{b′} are each, independently, H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl, wherein the C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl is optionally substituted with OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;
R^{c} and R^{d} are each, independently, H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl, wherein the C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl is optionally substituted with OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;
or R^{c} and R^{d} together with the N atom to which they are attached form a 4-, 5-, 6- or 7-membered heterocycloalkyl group; and
R^{c′} and R^{d′} are each, independently, H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl, wherein the C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl is optionally substituted with OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;
or R^{c′} and R^{d′} together with the N atom to which they are attached form a 4-, 5-, 6- or 7-membered heterocycloalkyl group.

In some embodiments, R⁴ is halo, C₁₋₆ alkyl, aryl, heteroaryl, cycloalkyl, heterocycloalkyl, arylalkyl, heteroarylalkyl, cycloalkylalkyl or heterocycloalkylalkyl, wherein the C₁₋₆ alkyl, aryl, heteroaryl, cycloalkyl, heterocycloalkyl, arylalkyl, heteroarylalkyl, cycloalkylalkyl or heterocycloalkylalkyl is optionally substituted by 1, 2 or 3 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl.

In some embodiments, R⁴ is C₁₋₆ alkyl, aryl, heteroaryl, cycloalkyl, heterocycloalkyl, arylalkyl, heteroarylalkyl, cycloalkylalkyl or heterocycloalkylalkyl.

In some embodiments, R^{2a} and R^{2b} are each, independently, H, halo, C₁₋₄ alkyl, C₁₋₄ haloalkyl, C₂₋₆ alkenyl, C₂₋₆ alkynyl, aryl, cycloalkyl, heteroaryl, heterocycloalkyl, NO₂, OR^{a′}, SR^{a′}, OC(O)R^{b′}, OC(O)NR^{c′}R^{d′}, S(O)R^{b′}, S(O)NR^{c′}R^{d′}, S(O)₂R^{b′}, or S(O)₂NR^{c′}R^{d′}.

In some embodiments, R^{2a} and R^{2b} are each, independently, H, halo, C₁₋₄ alkyl, C₁₋₄ haloalkyl, C₂₋₆ alkenyl, C₂₋₆ alkynyl, aryl, cycloalkyl, heteroaryl or heterocycloalkyl.

In some embodiments, R^{2a} and R^{2b} are each, independently, H or C₁₋₄ alkyl.

In some embodiments, R^{2a} and R^{2b} are both H.

In some embodiments, Q is aryl or heteroaryl, each optionally substituted by 1, 2, 3, 4 or 5 Cy¹ or R^{Q}.

In some embodiments, Q is aryl optionally substituted by 1, 2, 3, 4 or 5 Cy¹ or R^{Q}.

In some embodiments, Q is aryl optionally substituted by 1, 2 or 3 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl or heterocycloalkylalkyl.

In some embodiments, Q is aryl substituted by Cy¹ and optionally substituted by 1, 2 or 3 R^{Q}.

In some embodiments, Q is aryl substituted by Cy¹ and optionally substituted by 1, 2 or 3 R^{Q}; and Cy¹ is aryl or heteroaryl, each optionally substituted by 1, 2, 3, 4 or 5 substituents independently selected from halo, CN, OH, C₁₋₆ alkoxy, C₁₋₆haloalkoxy, C₁₋₆haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl.

In some embodiments, Q is phenyl wherein the phenyl is meta-substituted by Cy¹; and Cy¹ is aryl or heteroaryl, each optionally substituted by 1, 2, 3, 4 or 5 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl.

In some embodiments, Q is phenyl wherein the phenyl is meta-substituted by Cy¹; and Cy¹ is aryl optionally substituted by 1, 2, 3, 4 or 5 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl.

In some embodiments, R³ is C₁₋₁₀ alkyl, C₂₋₁₀ alkenyl, C₂₋₁₀ alkynyl, cycloalkyl, heterocycloalkyl, arylalkyl, heteroarylalkyl, cycloalkylalkyl or heterocycloalkylalkyl, wherein the C₁₋₁₀ alkyl, C₂₋₁₀ alkenyl, C₂₋₁₀ alkynyl, cycloalkyl, heterocycloalkyl, arylalkyl, heteroarylalkyl, cycloalkylalkyl or heterocycloalkylalkyl are each optionally substituted with 1, 2, 3, 4 or 5 A².

In some embodiments, R³ is C₁₋₁₀ alkyl, arylalkyl, heteroarylalkyl, cycloalkylalkyl or heterocycloalkylalkyl, wherein the C₁₋₁₀ alkyl, arylalkyl, heteroarylalkyl, cycloalkylalkyl or heterocycloalkylalkyl are each optionally substituted with 1, 2, 3, 4 or 5 A².

In some embodiments, R³ is C₁₋₁₀ alkyl, arylalkyl, heteroarylalkyl, cycloalkylalkyl or heterocycloalkylalkyl, wherein the C₁₋₁₀ alkyl, arylalkyl, heteroarylalkyl, cycloalkylalkyl or heterocycloalkylalkyl are each optionally substituted with 1, 2 or 3 A²; and A² is halo, CN, NO₂, OR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, NR^{c}S(O)₂R^{b}, S(O)₂R^{b}, S(O)₂NR^{c}R^{d}, C₁₋₄ alkoxy, C₁₋₄ haloalkoxy, amino, C₁₋₄ alkylamino, C₂₋₈ dialkylamino, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl or heterocycloalkyl, wherein each of the C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl or heterocycloalkyl is optionally substituted by 1, 2, 3, 4 or 5 halo, C₁₋₆ alkyl, C₂₋₆ alkenyl, C₂₋₆ alkynyl, C₁₋₄ haloalkyl, aryl, cycloalkyl, heteroaryl, heterocycloalkyl, CN, NO₂, OR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, NR^{c}S(O)₂R^{b}, S(O)R^{b}, S(O)₂R^{b}, or S(O)₂NR^{c}R^{d}.

In some embodiments, R³ is C₁₋₁₀ alkyl, arylalkyl, heteroarylalkyl, cycloalkylalkyl or heterocycloalkylalkyl.

In some embodiments, R³ is C₁₋₁₀ alkyl.

Also provided herein are novel compounds of structural formula VIII:

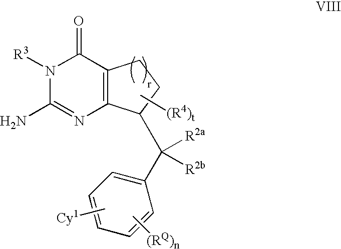

wherein:
R⁴ is C₁₋₆ alkyl, aryl, heteroaryl, cycloalkyl, heterocycloalkyl, arylalkyl, heteroarylalkyl, cycloalkylalkyl or heterocycloalkylalkyl;
R^{2a} and R^{2b} are each, independently, H or C₁₋₄ alkyl;
R³ is C₁₋₁₀ alkyl, arylalkyl, heteroarylalkyl, cycloalkylalkyl or heterocycloalkylalkyl, wherein the C₁₋₁₀ alkyl, arylalkyl, heteroarylalkyl, cycloalkylalkyl or heterocycloalkylalkyl are each optionally substituted with 1, 2, 3, 4 or 5 halo, CN, NO₂, OR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, NR^{c}S(O)₂R^{b}, S(O)₂R^{b}, S(O)₂NR^{c}R^{d}, C₁₋₄ alkoxy, C₁₋₄ haloalkoxy, amino, C₁₋₄ alkylamino, C₂₋₈ dialkylamino, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl or heterocycloalkyl;
R^{Q} is halo, CN, C₁₋₄ alkoxy, C₁₋₄ haloalkoxy, C₁₋₆ alkyl, C₂₋₆ alkenyl, C₂₋₆ alkynyl, arylalkyl, cycloalkylalkyl, heteroarylalkyl or heterocycloalkylalkyl;
Cy¹ is aryl or heteroaryl, each optionally substituted by 1, 2, 3, 4 or 5 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl;
n is 0 or 1;
r is 1 or 2; and
t is 0, 1, 2 or 3.

Also provided herein are novel compounds of structural formula IX:

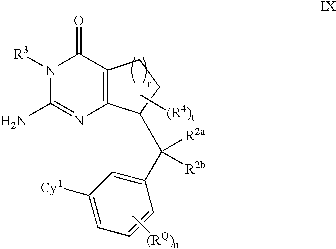

wherein:
R⁴ is C₁₋₆ alkyl, aryl, heteroaryl, cycloalkyl, heterocycloalkyl, arylalkyl, heteroarylalkyl, cycloalkylalkyl or heterocycloalkylalkyl;
R^{2a} and R^{2b} are each, independently, H or C₁₋₄ alkyl;
R³ is C₁₋₁₀ alkyl, arylalkyl, heteroarylalkyl, cycloalkylalkyl or heterocycloalkylalkyl, wherein the C₁₋₁₀ alkyl, arylalkyl, heteroarylalkyl, cycloalkylalkyl or heterocycloalkylalkyl are each optionally substituted with 1, 2, 3, 4 or 5 halo, CN, NO₂, OR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, NR^{c}S(O)₂R^{b}, S(O)₂R^{b}, S(O)₂NR^{c}R^{d}, C₁₋₄ alkoxy, C₁₋₄haloalkoxy, amino, C₁₋₄ alkylamino, C₂₋₈ dialkylamino, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl or heterocycloalkyl;
R^{Q} is halo, CN, C₁₋₄ alkoxy, C₁₋₄ haloalkoxy, C₁₋₆ alkyl, C₂₋₆ alkenyl, C₂₋₆ alkynyl, arylalkyl, cycloalkylalkyl, heteroarylalkyl or heterocycloalkylalkyl;
Cy¹ is aryl or heteroaryl, each optionally substituted by 1, 2, 3, 4 or 5 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl;
n is 0 or 1;
r is 1 or 2; and
t is 0, 1, 2 or 3.

In some embodiments, n is 0.

In some embodiments, n is 0; and Cy¹ is phenyl optionally substituted by 1, 2, 3, 4 or 5 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl.

Also provided herein are novel compounds of structural formula X:

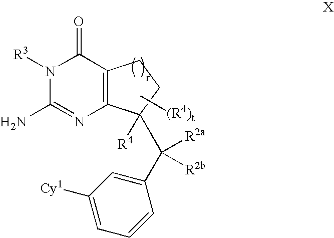

wherein:
R⁴ is C₁₋₆ alkyl, aryl, heteroaryl, cycloalkyl, heterocycloalkyl, arylalkyl, heteroarylalkyl, cycloalkylalkyl or heterocycloalkylalkyl;
R^{2a} and R^{2b} are each, independently, H or C₁₋₄ alkyl;
R³ is C₁₋₁₀ alkyl, arylalkyl, heteroarylalkyl, cycloalkylalkyl or heterocycloalkylalkyl, wherein the C₁₋₁₀ alkyl, arylalkyl, heteroarylalkyl, cycloalkylalkyl or heterocycloalkylalkyl are each optionally substituted with 1, 2, 3, 4 or 5 halo, CN, NO₂, OR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, NR^{c}S(O)₂R^{b}, S(O)₂R^{b}, S(O)₂NR^{c}R^{d}, C₁₋₄alkoxy, C₁₋₄haloalkoxy, amino, C₁₋₄ alkylamino, C₂₋₈ dialkylamino, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl or heterocycloalkyl;
Cy¹ is aryl or heteroaryl, each optionally substituted by 1, 2, 3, 4 or 5 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl;
r is 1 or 2; and
t is 0, 1 or 2.

In some embodiments, Cy¹ is phenyl optionally substituted by 1, 2, 3, 4 or 5 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl.

Also provided herein are novel compounds of structural formula XI:

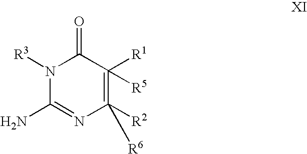

or a pharmaceutically acceptable salt, tautomer or in vivo-hydrolysable precursors thereof, wherein:
R¹ is C₁₋₆ alkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl, each optionally substituted by 1, 2, 3, 4 or 5 A¹;
R² is —(CR^{2a}R^{2b})ₘ-Q;
R³ is H, C(O)R^{a}, C(O)OR^{b}, C(O)NR^{c}R^{d}, S(O)R^{a}, S(O)₂R^{a}, C₁₋₁₀ alkyl, C₂₋₁₀ alkenyl, C₂₋₁₀ alkynyl, cycloalkyl, heterocycloalkyl, arylalkyl, heteroarylalkyl, cycloalkylalkyl or heterocycloalkylalkyl, wherein the C₁₋₁₀ alkyl, C₂₋₁₀ alkenyl, C₂₋₁₀ alkynyl, cycloalkyl, heterocycloalkyl, arylalkyl, heteroarylalkyl, cycloalkylalkyl or heterocycloalkylalkyl are each optionally substituted with 1, 2, 3, 4 or 5 A²;
R⁵ is H, C₁₋₆ alkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl, wherein each of the C₁₋₆ alkyl, aryl, heteroaryl, cycloalkyl, heterocycloalkyl, arylalkyl, heteroarylalkyl, cycloalkylalkyl or heterocycloalkylalkyl is optionally substituted by 1, 2, 3, 4 or 5 A⁴;
R⁶ is H, C₁₋₆ alkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl, wherein each of the C₁₋₆ alkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl is optionally substituted by 1, 2, 3, 4 or 5 A⁵;
R^{2a} and R^{2b} are each, independently, H, halo, C₁₋₄ alkyl, C₁₋₄ haloalkyl, C₂₋₆ alkenyl, C₂₋₆ alkynyl, aryl, cycloalkyl, heteroaryl, heterocycloalkyl, CN, NO₂, OR^{a′}, SR^{a′}, C(O)R^{b′}, C(O)NR^{c′}R^{d′}, C(O)OR^{a′}, OC(O)R^{b′}, OC(O)NR^{c′}R^{d′}, NR^{c′}R^{d′}, NR^{c′}C(O)R^{d′}, NR^{c′}C(O)OR^{a′}, NR^{c′}S(O)₂R^{b′}, S(O)R^{b′}, S(O)NR^{c′}R^{d′}, S(O)₂R^{b′}, or S(O)₂NR^{c′}R^{d′};
m is 0, 1, 2, 3 or 4;
Q is aryl, cycloalkyl, heteroaryl or heterocycloalkyl, each optionally substituted by 1, 2, 3, 4 or 5 Cy¹ or R^{Q};
Cy¹ is aryl, heteroaryl, cycloalkyl, or heterocycloalkyl, each optionally substituted with 1, 2, 3, 4 or 5 A³;
A¹, A², A³, A⁴, and A⁵ are each, independently, halo, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, NR^{c}S(O)R^{b}, NR^{c}S(O)₂R^{b}, S(O)R^{b}, S(O)NR^{c}R^{d}, S(O)₂R^{b}, S(O)₂NR^{c}R^{d}, C₁₋₄ alkoxy, C₁₋₄ haloalkoxy, amino, C₁₋₄ alkylamino, C₂₋₈ dialkylamino, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl or heterocycloalkyl, wherein each of the C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl or heterocycloalkyl is optionally substituted by 1, 2, 3, 4 or 5 halo, C₁₋₆ alkyl, C₂₋₆ alkenyl, C₂₋₆ alkynyl, C₁₋₄ haloalkyl, aryl, cycloalkyl, heteroaryl, heterocycloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, NR^{c}S(O)R^{b}, NR^{c}S(O)₂R^{b}, S(O)R^{b}, S(O)NR^{c}R^{d}, S(O)₂R^{b}, or S(O)₂NR^{c}R^{d};
R^{Q} is halo, CN, NO₂, OR^{a}, SR^{a}, C(O)R^{b}, C(O)NR^{c}R^{d}, C(O)OR^{a}, OC(O)R^{b}, OC(O)NR^{c}R^{d}, S(O)R^{b}, S(O)NR^{c}R^{d}, S(O)₂R^{b}, S(O)₂NR^{c}R^{d}, C₁₋₄ alkoxy, C₁₋₄haloalkoxy, C₁₋₆ alkyl, C₂₋₆ alkenyl, C₂₋₆ alkynyl, arylalkyl, cycloalkylalkyl, heteroarylalkyl or heterocycloalkylalkyl, wherein each of the C₁₋₆ alkyl, C₂₋₆ alkenyl, C₂₋₆ alkynyl, arylalkyl, cycloalkylalkyl, heteroarylalkyl or heterocycloalkylalkyl is optionally substituted by 1, 2, 3, 4 or 5 halo, C₁₋₆ alkyl, C₂₋₆ alkenyl, C₂₋₆ alkynyl, C₁₋₄ haloalkyl, aryl, cycloalkyl, heteroaryl, heterocycloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, NR^{c}S(O)R^{b}, NR^{c}S(O)₂R^{b}, S(O)R^{b}, S(O)NR^{c}R^{d}, S(O)₂R^{b}, or S(O)₂NR^{c}R^{d};
R^{a} and R^{a′} are each, independently, H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl, wherein the C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl is optionally substituted with OH, amino, halo, C₁₋₆ alkyl, C₁₋₆ haloalkyl, aryl, arylalkyl, heteroaryl, heteroarylalkyl, cycloalkyl or heterocycloalkyl;
R^{b} and R^{b′} are each, independently, H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl, wherein the C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl is optionally substituted with OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;
R^{c} and R^{d} are each, independently, H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl, wherein the C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl is optionally substituted with OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;
or R^{c} and R^{d} together with the N atom to which they are attached form a 4-, 5-, 6- or 7-membered heterocycloalkyl group; and
R^{c′} and R^{d′} are each, independently, H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl, wherein the C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl is optionally substituted with OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;
or R^{c′} and R^{d′} together with the N atom to which they are attached form a 4-, 5-, 6- or 7-membered heterocycloalkyl group.

In some embodiments, R¹ is C₁₋₆ alkyl, cycloalkyl, heterocycloalkyl, arylalkyl, heteroarylalkyl, cycloalkylalkyl or heterocycloalkylalkyl, each optionally substituted by 1, 2 or 3 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl.

In some embodiments, R¹ is C₁₋₆ alkyl, cycloalkyl, heterocycloalkyl, arylalkyl, heteroarylalkyl, cycloalkylalkyl or heterocycloalkylalkyl.

In some embodiments, R^{2a} and R^{2b} are each, independently, H, halo, C₁₋₄ alkyl, C₁₋₄, haloalkyl, C₂₋₆ alkenyl, C₂₋₆ alkynyl, aryl, cycloalkyl, heteroaryl or heterocycloalkyl.

In some embodiments, R^{2a} and R^{2b} are each, independently, H or C₁₋₄ alkyl.

In some embodiments, R^{2a} and R^{2b} are both H.

In some embodiments, m is 0.

In some embodiments, Q is aryl or heteroaryl, each optionally substituted by 1, 2, 3, 4 or 5 Cy¹ or R^{Q}.

In some embodiments, Q is aryl optionally substituted by 1, 2, 3, 4 or 5 Cy¹ or R^{Q}.

In some embodiments, Q is aryl optionally substituted by 1, 2 or 3 R^{Q}.

In some embodiments, Q is aryl substituted by Cy¹ and optionally substituted by 1, 2 or 3 R^{Q}.

In some embodiments, Q is aryl substituted by Cy¹ and optionally substituted by 1, 2 or 3 R^{Q}; and Cy¹ is aryl or heteroaryl, each optionally substituted by 1, 2, 3, 4 or 5 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl.

In some embodiments, R^{Q} is halo, CN, OH, C₁₋₆ alkoxy, C₁₋₆ haloalkoxy, C₁₋₆haloalkyl, C₁₋₆ alkyl, C₂₋₆ alkenyl, C₂₋₆ alkynyl, arylalkyl, cycloalkylalkyl, heteroarylalkyl or heterocycloalkylalkyl.

In some embodiments, Q is phenyl wherein the phenyl is meta-substituted by Cy¹; and Cy¹ is aryl or heteroaryl, each optionally substituted by 1, 2, 3, 4 or 5 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl.

In some embodiments, Q is phenyl wherein the phenyl is meta-substituted by Cy¹; and Cy¹ is aryl optionally substituted by 1, 2, 3, 4 or 5 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alynyl, arylalkyl, cycloalkylalkyl, heteroarylalkyl, heterocycloalkylalkyl, aryl, cycloalkyl, heteroaryl and heterocycloalkyl.

In some embodiments, R³ is H, C₁₋₁₀ alkyl, C₂₋₁₀ alkenyl, C₂₋₁₀ alkynyl, cycloalkyl, heterocycloalkyl, arylalkyl, heteroarylalkyl, cycloalkylalkyl or heterocycloalkylalkyl, wherein the C₁₋₁₀ alkyl, C₂₋₁₀ alkenyl, C₂₋₁₀ alkynyl, cycloalkyl, heterocycloalkyl, arylalkyl, heteroarylalkyl, cycloalkylalkyl or heterocycloalkylalkyl are each optionally substituted with 1, 2, 3, 4 or 5 A².

In some embodiments, R³ is H, C₁₋₁₀ alkyl, arylalkyl, heteroarylalkyl, cycloalkylalkyl or heterocycloalkylalkyl, wherein the C₁₋₁₀ alkyl, arylalkyl, heteroarylalkyl, cycloalkylalkyl or heterocycloalkylalkyl are each optionally substituted with 1, 2, 3, 4 or 5 A².

In some embodiments, R³ is H, C₁₋₁₀ alkyl, arylalkyl, heteroarylalkyl, cycloalkylalkyl or heterocycloalkylalkyl, wherein each of the C₁₋₁₀ alkyl, arylalkyl, heteroarylalkyl, cycloalkylalkyl or heterocycloalkylalkyl are each optionally substituted with 1, 2 or 3 A²; and A² is halo, CN, NO₂, OR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, NR^{c}S(O)₂R^{b}, S(O)₂R^{b}, S(O)₂NR^{c}R^{d}, C₁₋₄ alkoxy, C₁₋₄ haloalkoxy, amino, C₁₋₄ alkylamino, C₂₋₈ dialkylamino, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl or heterocycloalkyl, wherein each of the C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl or heterocycloalkyl is optionally substituted by 1, 2, 3, 4 or 5 halo, C₁₋₆ alkyl, C₂₋₆ alkenyl, C₂₋₆ alkynyl, C₁₋₄ haloalkyl, aryl, cycloalkyl, heteroaryl, heterocycloalkyl, CN, NO₂, OR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, NR^{c}S(O)₂R^{b}, S(O)R^{b}, S(O)₂R^{b}, or S(O)₂NR^{c}R^{d}.

In some embodiments, R³ is H, C₁₋₁₀ alkyl, arylalkyl, heteroarylalkyl, cycloalkylalkyl or heterocycloalkylalkyl.

In some embodiments, R³ is H or C₁₋₁₀ alkyl.

In some embodiments, R⁵ is H.

In some embodiments, R⁶ is C₁₋₁₀ alkyl.

In some embodiments, R¹ is C₁₋₆ alkyl, cycloalkyl, heterocycloalkyl, arylalkyl, heteroarylalkyl, cycloalkylalkyl or heterocycloalkylalkyl; R^{2a} and R^{2b} are each, independently, H or C₁₋₄ alkyl; Q is aryl optionally substituted by 1, 2 or 3 R^{Q}; m is 0, 1 or 2; R³ is H, C₁₋₁₀ alkyl, arylalkyl, heteroarylalkyl, cycloalkylalkyl or heterocycloalkylalkyl, wherein the C₁₋₁₀ alkyl, arylalkyl, heteroarylalkyl, cycloalkylalkyl or heterocycloalkylalkyl are each optionally substituted with 1, 2, 3, 4 or 5 A²; R⁵ is H; and R⁶ is C₁₋₁₀ alkyl.

In some embodiments, R¹ is C₁₋₆ alkyl, cycloalkyl, heterocycloalkyl, arylalkyl, heteroarylalkyl, cycloalkylalkyl or heterocycloalkylalkyl; R^{2a} and R^{2b} are each, independently, H or C₁₋₄ alkyl; Q is phenyl optionally substituted by 1, 2 or 3 halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, or heterocycloalkylalkyl; m is 0, 1 or 2; R³ is C₁₋₁₀ alkyl, arylalkyl, heteroarylalkyl, cycloalkylalkyl or heterocycloalkylalkyl, wherein the C₁₋₁₀ alkyl, arylalkyl, heteroarylalkyl, cycloalkylalkyl or heterocycloalkylalkyl are each optionally substituted with 1, 2, 3, 4 or 5 halo, CN, NO₂, OR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, NR^{c}S(O)₂R^{b}, S(O)₂R^{b}, S(O)₂NR^{c}R^{d}, C₁₋₄ alkoxy, C₁₋₄ haloalkoxy, amino, C₁₋₄ alkylamino, C₂₋₈ dialkylamino, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl or heterocycloalkyl; R⁵ is H; and R⁶ is C₁₋₁₀ alkyl.

In some embodiments, m is 0.

Also provided herein are novel compounds of structural formula XII:

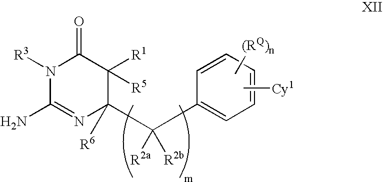

wherein:
R¹ is C₁₋₆ alkyl, cycloalkyl, heterocycloalkyl, arylalkyl, heteroarylalkyl, cycloalkylalkyl or heterocycloalkylalkyl;
R^{2a} an R^{2b} are each, independently, H or C₁₋₄ alkyl;
R³ is C₁₋₁₀ alkyl, arylalkyl, heteroarylalkyl, cycloalkylalkyl or heterocycloalkylalkyl, wherein the C₁₋₁₀ alkyl, arylalkyl, heteroarylalkyl, cycloalkylalkyl or heterocycloalkylalkyl are each optionally substituted with 1, 2, 3, 4 or 5 halo, CN, NO₂, OR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, NR^{c}S(O)₂R^{b}, S(O)₂R^{b}, S(O)₂NR^{c}R^{d}, C₁₋₄ alkoxy, C₁₋₄ haloalkoxy, amino, C₁₋₄ alkylamino, C₂₋₈ dialkylamino, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl or heterocycloalkyl;

### R⁵ is H;

R⁶ is C₁₋₁₀ alkyl;
R^{Q} is halo, CN, C₁₋₄ alkoxy, C₁₋₄ haloalkoxy, C₁₋₆ alkyl, C₂₋₆ alkenyl, C₂₋₆ alkynyl, arylalkyl, cycloalkylalkyl, heteroarylalkyl or heterocycloalkylalkyl;
Cy¹ is aryl or heteroaryl, each optionally substituted by 1, 2, 3, 4 or 5 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl;
m is 0, 1, or 2; and
n is 0 or 1.

Also provided herein are novel compounds of structural formula XIII:

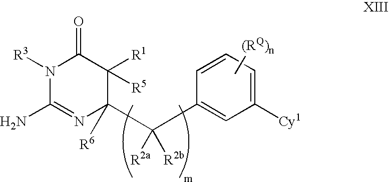

wherein:
R¹ is C₁₋₆ alkyl, cycloalkyl, heterocycloalkyl, arylalkyl, heteroarylalkyl, cycloalkylalkyl or heterocycloalkylalkyl;
R^{2a} and R^{2b} are each, independently, H or C₁₋₄ alkyl;
R³ is C₁₋₁₀ alkyl, arylalkyl, heteroarylalkyl, cycloalkylalkyl or heterocycloalkylalkyl, wherein the C₁₋₁₀ alkyl, arylalkyl, heteroarylalkyl, cycloalkylalkyl or heterocycloalkylalkyl are each optionally substituted with 1, 2, 3, 4 or 5 halo, CN, NO₂, OR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, NR^{c}S(O)₂R^{b}, S(O)₂R^{b}, S(O)₂NR^{c}R^{d}, C₁₋₄ alkoxy, C₁₋₄ haloalkoxy, amino, C₁₋₄ alkylamino, C₂₋₈ dialkylamino, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl or heterocycloalkyl;

### R⁵ is H;

R⁶ is C₁₋₁₀ alkyl;
R^{Q} is halo, CN, C₁₋₄ alkoxy, C₁₋₄ haloalkoxy, C₁₋₆ alkyl, C₂₋₆ alkenyl, C₂₋₆ alkynyl, arylalkyl, cycloalkylalkyl, heteroarylalkyl or heterocycloalkylalkyl;
Cy¹ is aryl or heteroaryl, each optionally substituted by 1, 2, 3, 4 or 5 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl;
m is 0, 1, or 2; and
n is 0 or 1.

The present invention further provides compositions comprising a compound of any of the formulas described herein, or a pharmaceutically acceptable salt, tautomer or in vivo-hydrolysable precursor thereof, and at least one pharmaceutically acceptable carrier, diluent or excipient.

The present invention further provides methods of modulating activity of BACE comprising contacting the BACE with a compound of any of the formulas described herein, or a pharmaceutically acceptable salt, tautomer or in vivo-hydrolysable precursor thereof.

The present invention further provides methods of treating or preventing an Aβ-related pathology in a patient, comprising administering to the patient a therapeutically effective amount of a compound of any of the formulas described herein, or a pharmaceutically acceptable salt, tautomer or in vivo-hydrolysable precursor thereof.

The present invention further provides a compound of any of the formulas described herein, or a pharmaceutically acceptable salt, tautomer or in vivo-hydrolysable precursor thereof, described herein for use as a medicament.

The present invention further provides a compound of any of the formulas described herein, or a pharmaceutically acceptable salt, tautomer or in vivo-hydrolysable precursor thereof, described herein for the manufacture of a medicament.

### DETAILED DESCRIPTION OF THE INVENTION

Provided herein are novel compounds of structural formula I:

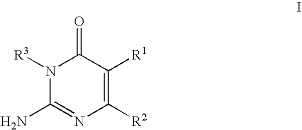

or a pharmaceutically acceptable salt, tautomer or in vivo-hydrolysable precursor thereof.

In some embodiments, R¹ is halo, CN, OR^{a}, SR^{a}, C(O)R^{b}, C(O)NR^{c}R^{d}, C(O)OR^{a}, OC(O)R^{b}, OC(O)NR^{c}R^{d}, S(O)R^{b}, S(O)NR^{c}R^{d}, S(O)₂R^{b}, S(O)₂NR^{c}R^{d}, C₁₋₆ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl, or any subgroup thereof, wherein each of the C₁₋₆ alkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl is optionally substituted by 1, 2, 3, 4 or 5 A¹, or any subgroup thereof. In some embodiments, R¹ is halo, C₁₋₆ alkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl, wherein each of the C₁₋₆ alkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl is optionally substituted by 1, 2 or 3 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl. In some embodiments, R¹ is halo, C₁₋₆ alkyl, aryl, heteroaryl, cycloalkyl, heterocycloalkyl, arylalkyl, heteroarylalkyl, cycloalkylalkyl or heterocycloalkylalkyl. In some embodiments, R¹ is halo, C₁₋₆ alkyl, aryl, heteroaryl, cycloalkyl, heterocycloalkyl, arylalkyl, heteroarylalkyl, cycloalkylalkyl or heterocycloalkylalkyl.

In some embodiments, R^{2a} and R^{2b} are each, independently, H, halo, C₁₋₄ alkyl, C₁₋₄ haloalkyl, C₂₋₆ alkenyl, C₂₋₆ alkynyl, aryl, cycloalkyl, heteroaryl, heterocycloalkyl, NO₂, OR^{a′}, SR^{a′}, OC(O)R^{b′}, OC(O)NR^{c′}R^{d′}, S(O)R^{b′}, S(O)NR^{c′}R^{d′}, S(O)₂R^{b′}, or S(O)₂NR^{c′}R^{d′}, or any subgroup thereof.

In some embodiments, R² is —(CR^{2a}R^{2b})₂-Q.

In some embodiments, R³ is H, C(O)R^{a}, C(O)OR^{b}, C(O)NR^{c}R^{d}, S(O)R^{a}, S(O)₂R^{a}, C₁₋₁₀ alkyl, C₂₋₁₀ alkenyl, C₂₋₁₀ alkynyl, cycloalkyl, heterocycloalkyl, arylalkyl, heteroarylalkyl, cycloalkylalkyl or heterocycloalkylalkyl, or any subgroup thereof, wherein the C₁₋₁₀ alkyl, C₂₋₁₀ alkenyl, C₂₋₁₀ alkynyl, cycloalkyl, heterocycloalkyl, arylalkyl, heteroarylalkyl, cycloalkylalkyl or heterocycloalkylalkyl are each optionally substituted with 1, 2, 3, 4 or 5 A². In some embodiments, R³ is C₁₋₁₀ alkyl, C₂₋₁₀ alkenyl, C₂₋₁₀ alkynyl, cycloalkyl, heterocycloalkyl, arylalkyl, heteroarylalkyl, cycloalkylalkyl or heterocycloalkylalkyl, wherein the C₁₋₁₀ alkyl, C₂₋₁₀ alkenyl, C₂₋₁₀ alkynyl, cycloalkyl, heterocycloalkyl, arylalkyl, heteroarylalkyl, cycloalkylalkyl or heterocycloalkylalkyl are each optionally substituted with 1, 2, 3, 4 or 5 A². In some embodiments, R³ is C₁₋₁₀ alkyl, arylalkyl, heteroarylalkyl, cycloalkylalkyl or heterocycloalkylalkyl, wherein the C₁₋₁₀ alkyl, arylalkyl, heteroarylalkyl, cycloalkylalkyl or heterocycloalkylalkyl are each optionally substituted with 1, 2, 3, 4 or 5 A². In some embodiments, R³ is C₁₋₁₀ alkyl, arylalkyl, heteroarylalkyl, cycloalkylalkyl or heterocycloalkylalkyl, wherein the C₁₋₁₀ alkyl, arylalkyl, heteroarylalkyl, cycloalkylalkyl or heterocycloalkylalkyl are each optionally substituted with 1, 2 or 3 A². In some embodiments, R³ is C₁₋₁₀ alkyl, arylalkyl, heteroarylalkyl, cycloalkylalkyl or heterocycloalkylalkyl, wherein the C₁₋₁₀ alkyl, arylalkyl, heteroarylalkyl, cycloalkylalkyl or heterocycloalkylalkyl are each optionally substituted with 1, 2, 3, 4 or 5 halo, CN, NO₂, OR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, NR^{c}S(O)₂R^{b}, S(O)₂R^{b}, S(O)₂NR^{c}R^{d}, C₁₋₄ alkoxy, C₁₋₄ haloalkoxy, amino, C₁₋₄ alkylamino, C₂₋₈ dialkylamino, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl or heterocycloalkyl.

In some embodiments, R^{2a} and R^{2b} are each, independently, H, halo, C₁₋₄ alkyl, C₁₋₄ haloalkyl, C₂₋₆ alkenyl, C₂₋₆ alkynyl, aryl, cycloalkyl, heteroaryl, heterocycloalkyl, CN, NO₂, OR^{a′}, SR^{a′}, C(O)R^{b′}, C(O)NR^{c′}R^{d}, C(O)OR^{a′}, OC(O)R^{b′}, OC(O)NR^{c′}R^{d′}, NR^{c′}R^{d′}, NR^{c′}C(O)R^{d′}, NR^{c′}C(O)OR^{a′}, NR^{c′}S(O)₂R^{b′}, S(O)R^{b}, S(O)NR^{c′}R^{d′}, S(O)₂R^{b′}, or S(O)₂NR^{c′}R^{d′}, or any subgroup thereof. In some embodiments, R^{2a} and R^{2b} are each, independently, H, halo, C₁₋₄ alkyl, C₁₋₄ haloalkyl, C₂₋₆ alkenyl, C₂₋₆ alkynyl, aryl, cycloalkyl, heteroaryl or heterocycloalkyl. In some embodiments, R^{2a} and R^{2b} are each, independently, H or C₁₋₄ alkyl. In some embodiments, R^{2a} and R^{2b} are both H.

In some embodiments, Q is aryl, cycloalkyl, heteroaryl or heterocycloalkyl, or any subgroup thereof, each optionally substituted by 1, 2, 3, 4 or 5 Cy¹ or R^{Q}. In some embodiments, Q is aryl or heteroaryl, each optionally substituted by 1, 2, 3, 4 or 5 Cy¹ or R^{Q}. In some embodiments, Q is aryl optionally substituted by 1, 2, 3, 4 or 5 Cy¹ or R^{Q}. In some embodiments, Q is aryl optionally substituted by 1, 2 or 3 R^{Q}. In some embodiments, Q is aryl substituted by Cy¹ and optionally substituted by 1, 2 or 3 R^{Q}. In some embodiments, Q is phenyl wherein the phenyl is meta-substituted by Cy¹. In some embodiments, Q is phenyl optionally substituted by 1, 2 or 3 halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, or heterocycloalkylalkyl. In some embodiments, Q is phenyl meta-substituted by halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, or heterocycloalkylalkyl.

In some embodiments, Cy¹ is aryl, heteroaryl, cycloalkyl, or heterocycloalkyl, or any subgroup thereof, each optionally substituted with 1, 2, 3, 4 or 5 A³. In some embodiments, Cy¹ is aryl or heteroaryl, each optionally substituted by 1, 2, 3, 4 or 5 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl. In some embodiments, Cy¹ is aryl optionally substituted by 1, 2, 3, 4 or 5 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl.

In some embodiments, A¹, A², and A³ are each, independently, halo, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, NR^{c}S(O)R^{b}, NR^{c}S(O)₂R^{b}, S(O)R^{b}, S(O)NR^{c}R^{d}, S(O)₂R^{b}, S(O)₂NR^{c}R^{d}, C₁₋₄ alkoxy, C₁₋₄ haloalkoxy, amino, C₁₋₄ alkylamino, C₂₋₈ dialkylamino, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl or heterocycloalkyl, or any subgroup thereof, wherein each of the C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl or heterocycloalkyl is optionally substituted by 1, 2, 3, 4 or 5 halo, C₁₋₆ alkyl, C₂₋₆ alkenyl, C₂₋₆ alkynyl, C₁₋₄ haloalkyl, aryl, cycloalkyl, heteroaryl, heterocycloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, NR^{c}S(O)R^{b}, NR^{c}S(O)₂R^{b}, S(O)R^{b}, S(O)NR^{c}R^{d}, S(O)₂R^{b}, or S(O)₂NR^{c}R^{d}, or any subgroup thereof. In some embodiments, A² is halo, CN, NO₂, OR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, NR^{c}S(O)₂R^{b}, S(O)₂R^{b}, S(O)₂NR^{c}R^{d}, C₁₋₄ alkoxy, C₁₋₄haloalkoxy, amino, C₁₋₄ alkylamino, C₂₋₈ dialkylamino, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl or heterocycloalkyl, wherein each of the C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl or heterocycloalkyl is optionally substituted by 1, 2, 3, 4 or 5 halo, C₁₋₆ alkyl, C₂₋₆ alkenyl, C₂₋₆ alkynyl, C₁₋₄ haloalkyl, aryl, cycloalkyl, heteroaryl, heterocycloalkyl, CN, NO₂, OR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, NR^{c}S(O)₂R^{b}, S(O)R^{b}, S(O)₂R^{b}, or S(O)₂NR^{c}R^{d}.

In some embodiments, R^{Q} is halo, CN, NO₂, OR^{a}, SR^{a}, C(O)R^{b}, C(O)NR^{c}R^{d}, C(O)OR^{a}, OC(O)R^{b}, OC(O)NR^{c}R^{d}, S(O)R^{b}, S(O)NR^{c}R^{d}, S(O)₂R^{b}, S(O)₂NR^{c}R^{d}, C₁₋₄ alkoxy, C₁₋₄ haloalkoxy, C₁₋₆ alkyl, C₂₋₆ alkenyl, C₂₋₆ alkynyl, arylalkyl, cycloalkylalkyl, heteroarylalkyl or heterocycloalkylalkyl, or any subgroup thereof, wherein each of the C₁₋₆ alkyl, C₂₋₆ alkenyl, C₂₋₆ alkynyl, arylalkyl, cycloalkylalkyl, heteroarylalkyl or heterocycloalkylalkyl is optionally substituted by 1, 2, 3, 4 or 5 halo, C₁₋₆ alkyl, C₂₋₆ alkenyl, C₂₋₆ alkynyl, C₁₋₄ haloalkyl, aryl, cycloalkyl, heteroaryl, heterocycloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, NR^{c}S(O)R^{b}, NR^{c}S(O)₂R^{b}, S(O)R^{b}, S(O)NR^{c}R^{d}, S(O)₂R^{b}, or S(O)₂NR^{c}R^{d}, or any subgroup thereof.

In some embodiments, R^{a} and R^{a′} are each, independently, H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl, or any subgroup thereof, wherein the C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl is optionally substituted with OH, amino, halo, C₁₋₆ alkyl, C₁₋₆ haloalkyl, aryl, arylalkyl, heteroaryl, heteroarylalkyl, cycloalkyl or heterocycloalkyl, or any subgroup thereof.

In some embodiments, R^{b} and R^{b′} are each, independently, H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl, or any subgroup thereof, wherein the C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl is optionally substituted with OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl, or any subgroup thereof.

In some embodiments, R^{c} and R^{d} are each, independently, H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl, or any subgroup thereof, wherein the C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl is optionally substituted with OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl, or any subgroup thereof.

In some embodiments, R^{c} and R^{d} together with the N atom to which they are attached form a 4-, 5-, 6- or 7-membered heterocycloalkyl group.

In some embodiments, R^{c′} and R^{d′} are each, independently, H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl, or any subgroup thereof, wherein the C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl is optionally substituted with OH, amino, halo, C₁₋₆ alkyl, C₁₋₆ haloalkyl, C₁₋₆haloalkyl, aryl, arylalkyl, heteroaryl, heteroarylalkyl, cycloalkyl or heterocycloalkyl, or any subgroup thereof.

In some embodiments, R^{c′} and R^{d′} together with the N atom to which they are attached form a 4-, 5-, 6- or 7-membered heterocycloalkyl group.

Also provided herein are novel compounds of structural formula II:

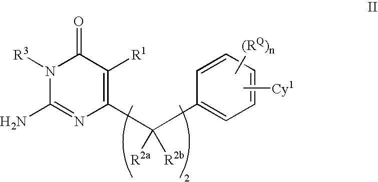

or a pharmaceutically acceptable salt, tautomer or in vivo-hydrolysable precursor thereof.

In some embodiments, R¹ is halo, C₁₋₆ alkyl, aryl, heteroaryl, cycloalkyl, heterocycloalkyl, arylalkyl, heteroarylalkyl, cycloalkylalkyl or heterocycloalkylalkyl, or any subgroup thereof.

In some embodiments, R^{2a} and R^{2b} are each, independently, H or C₁₋₄ alkyl, or any subgroup thereof.

In some embodiments, R³ is C₁₋₁₀ alkyl, arylalkyl, heteroarylalkyl, cycloalkylalkyl or heterocycloalkylalkyl, or any subgroup thereof, wherein the C₁₋₁₀ alkyl, arylalkyl, heteroarylalkyl, cycloalkylalkyl or heterocycloalkylalkyl are each optionally substituted with 1, 2, 3, 4 or 5 halo, CN, NO₂, OR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, NR^{c}S(O)₂R^{b}, S(O)₂R^{b}, S(O)₂NR^{c}R^{d}, C₁₋₄ alkoxy, C₁₋₄ haloalkoxy, amino, C₁₋₄ alkylamino, C₂₋₈ dialkylamino, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl or heterocycloalkyl, or any subgroup thereof.

In some embodiments, R^{Q} is halo, CN, C₁₋₄ alkoxy, C₁₋₄ haloalkoxy, C₁₋₆ alkyl, C₂₋₆ alkenyl, C₂₋₆ alkynyl, arylalkyl, cycloalkylalkyl, heteroarylalkyl or heterocycloalkylalkyl, or any subgroup thereof.

In some embodiments, Cy¹ is aryl or heteroaryl, each optionally substituted by 1, 2, 3, 4 or 5 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl, or any subgroup thereof.

In some embodiments, n is 0 or 1.

Also provided herein are novel compounds of structural formula III:

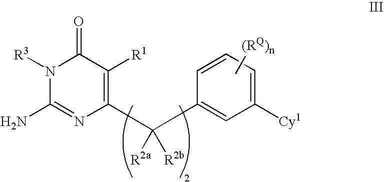

or a pharmaceutically acceptable salt, tautomer or in vivo-hydrolysable precursor thereof.

In some embodiments, R¹ is halo, C₁₋₆ alkyl, aryl, heteroaryl, cycloalkyl, heterocycloalkyl, arylalkyl, heteroarylalkyl, cycloalkylalkyl or heterocycloalkylalkyl, or any subgroup thereof.

In some embodiments, R^{2a} and R^{2b} are each, independently, H or C₁₋₄ alkyl, or any subgroup thereof.

In some embodiments, R³ is C₁₋₁₀ alkyl, arylalkyl, heteroarylalkyl, cycloalkylalkyl or heterocycloalkylalkyl, or any subgroup thereof, wherein the C₁₋₁₀ alkyl, arylalkyl, heteroarylalkyl, cycloalkylalkyl or heterocycloalkylalkyl are each optionally substituted with 1, 2, 3, 4 or 5 halo, CN, NO₂, OR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, NR^{c}S(O)₂R^{b}, S(O)₂R^{b}, S(O)₂NR^{c}R^{d}, C₁₋₄ alkoxy, C₁₋₄ haloalkoxy, amino, C₁₋₄ alkylamino, C₂₋₈ dialkylamino, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl or heterocycloalkyl, or any subgroup thereof.

In some embodiments, R^{Q} is halo, CN, C₁₋₄ alkoxy, C₁₋₄ haloalkoxy, C₁₋₆ alkyl, C₂₋₆ alkenyl, C₂₋₆ alkynyl, arylalkyl, cycloalkylalkyl, heteroarylalkyl or heterocycloalkylalkyl, or any subgroup thereof.

In some embodiments, Cy¹ is aryl or heteroaryl, each optionally substituted by 1, 2, 3, 4 or 5 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl, or any subgroup thereof. In some embodiments, Cy¹ is phenyl optionally substituted by 1, 2, 3, 4 or 5 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl.

In some embodiments, n is 0 or 1. In some embodiments, n is 0.

Also provided herein are novel compounds of structural formula IV:

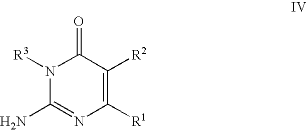

or a pharmaceutically acceptable salt, tautomer or in vivo-hydrolysable precursor thereof.

In some embodiments, R¹ is H, halo, CN, OR^{a}, SR^{a}, C(O)R^{b}, C(O)NR^{c}R^{d}, C(O)OR^{a}, OC(O)R^{b}, OC(O)NR^{c}R^{d}, S(O)R^{b}, S(O)NR^{c}R^{d}, S(O)₂R^{b}, S(O)₂NR^{c}R^{d}, C₁₋₆ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl, or any subgroup thereof, wherein each of the C₁₋₆ alkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl is optionally substituted by 1, 2, 3, 4 or 5 A¹, or any subgroup thereof. In some embodiments, R¹ is H, C₁₋₆ alkyl, aryl, heteroaryl, cycloalkyl, heterocycloalkyl, arylalkyl, heteroarylalkyl, cycloalkylalkyl or heterocycloalkylalkyl.

In some embodiments, R² is —(CR^{2a}R^{2b})₂-Q.

In some embodiments, R³ is C(O)R^{a}, C(O)OR^{b}, C(O)NR^{c}R^{d}, S(O)R^{a}, S(O)₂R^{a}, C₁₋₁₀ alkyl, C₂₋₁₀ alkenyl, C₂₋₁₀ alkynyl, cycloalkyl, heterocycloalkyl, arylalkyl, heteroarylalkyl, cycloalkylalkyl or heterocycloalkylalkyl, or any subgroup thereof, wherein the C₁₋₁₀ alkyl, C₂₋₁₀ alkenyl, C₂₋₁₀ alkynyl, cycloalkyl, heterocycloalkyl, arylalkyl, heteroarylalkyl, cycloalkylalkyl or heterocycloalkylalkyl are each optionally substituted with 1, 2, 3, 4 or 5 A², or any subgroup thereof. In some embodiments, R³ is C₁₋₁₀ alkyl, C₂₋₁₀ alkenyl, C₂₋₁₀ alkynyl, cycloalkyl, heterocycloalkyl, arylalkyl, heteroarylalkyl, cycloalkylalkyl or heterocycloalkylalkyl, wherein the C₁₋₁₀ alkyl, C₂₋₁₀ alkenyl, C₂₋₁₀ alkynyl, cycloalkyl, heterocycloalkyl, arylalkyl, heteroarylalkyl, cycloalkylalkyl or heterocycloalkylalkyl are each optionally substituted with 1, 2, 3, 4 or 5 A². In some embodiments, R³ is C₁₋₁₀ alkyl, arylalkyl, heteroarylalkyl, cycloalkylalkyl or heterocycloalkylalkyl, wherein the C₁₋₁₀ alkyl, arylalkyl, heteroarylalkyl, cycloalkylalkyl or heterocycloalkylalkyl are each optionally substituted with 1, 2, 3, 4 or 5 A². In some embodiments, R³ is C₁₋₁₀ alkyl, arylalkyl, heteroarylalkyl, cycloalkylalkyl or heterocycloalkylalkyl, wherein the C₁₋₁₀ alkyl, arylalkyl, heteroarylalkyl, cycloalkylalkyl or heterocycloalkylalkyl are each optionally substituted with 1, 2 or 3 A². In some embodiments, R³ is C₁₋₁₀ alkyl, arylalkyl, heteroarylalkyl, cycloalkylalkyl or heterocycloalkylalkyl. In some embodiments, R³ is C₁₋₁₀ alkyl.

In some embodiments, R^{2a} and R^{2b} are each, independently, H, halo, C₁₋₄ alkyl, C₁₋₄ haloalkyl, C₂₋₆ alkenyl, C₂₋₆ alkynyl, aryl, cycloalkyl, heteroaryl, heterocycloalkyl, CN, NO₂, OR^{a′}, SR^{a′}, C(O)R^{b′}, C(O)NR^{c′}R^{d′}, C(O)OR^{a′}, OC(O)R^{b′}, OC(O)NR^{c′}R^{d′}, NR^{c′}R^{d′}, NR^{c′}C(O)R^{d′}, NR^{c′}C(O)OR^{a′}, NR^{c′}S(O)₂R^{b′}, S(O)R^{b′}, S(O)NR^{c′}R^{d′}, S(O)₂R^{b′}, or S(O)₂NR^{c′}R^{d′}, or any subgroup thereof. In some embodiments, R^{2a} and R^{2b} are each, independently, H, halo, C₁₋₄ alkyl, C₁₋₄ haloalkyl, C₂₋₆ alkenyl, C₂₋₆ alkynyl, aryl, cycloalkyl, heteroaryl, heterocycloalkyl, NO₂, OR^{a′}, SR^{a′}, OC(O)R^{b′}, OC(O)NR^{c′}R^{d′}, S(O)R^{b′}, S(O)NR^{c′}R^{d′}, S(O)₂R^{b′}, or S(O)₂NR^{c′}R^{d′}. In some embodiments, R^{2a} and R^{2b} are each, independently, H, halo, C₁₋₄ alkyl, C₁₋₄ haloalkyl, C₂₋₆ alkenyl, C₂₋₆ alkyl, aryl, to cycloalkyl, heteroaryl or heterocycloalkyl. In some embodiments, R^{2a} and R^{2b} are each, independently, H or C₁₋₄ alkyl. In some embodiments, R^{2a} and R^{2b} are both H.

In some embodiments, Q is aryl, heteroaryl or cycloalkyl, each optionally substituted by 1, 2, 3, 4 or 5 Cy¹ or R^{Q}, or any subgroup thereof. In some embodiments, Q is aryl or heteroaryl, each optionally substituted by 1, 2, 3, 4 or 5 Cy¹ or R^{Q}. In some embodiments, Q is aryl optionally substituted by 1, 2, 3, 4 or 5 Cy¹ or R^{Q}. In some embodiments, Q is aryl optionally substituted by 1, 2 or 3 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl or heterocycloalkylalkyl. In some embodiments, Q is aryl substituted by Cy¹ and optionally substituted by 1, 2 or 3 R^{Q}. In some embodiments, Q is aryl substituted by Cy¹ and optionally substituted by 1, 2 or 3 R^{Q}. In some embodiments, Q is phenyl wherein the phenyl is meta-substituted by Cy¹.

In some embodiments, Cy¹ is aryl, heteroaryl or cycloalkyl, each optionally substituted with 1, 2, 3, 4 or 5 A³, or any subgroup thereof. In some embodiments, Cy¹ is aryl or heteroaryl, each optionally substituted by 1, 2, 3, 4 or 5 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl.

In some embodiments, A¹, A², and A³ are each, independently, halo, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, NR^{c}S(O)R^{b}, NR^{c}S(O)₂R^{b}, S(O)R^{b}, S(O)NR^{c}R^{d}, S(O)₂R^{b}, S(O)₂NR^{c}R^{d}, C₁₋₄ alkoxy, C₁₋₄ haloalkoxy, amino, C₁₋₄ alkylamino, C₂₋₈ dialkylamino, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl or heterocycloalkyl, or any subgroup thereof, wherein each of the C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl or heterocycloalkyl is optionally substituted by 1, 2, 3, 4 or 5 halo, C₁₋₆ alkyl, C₂₋₆ alkenyl, C₂₋₆ alkynyl, C₁₋₄ haloalkyl, aryl, cycloalkyl, heteroaryl, heterocycloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, NR^{c}S(O)R^{b}, NR^{c}S(O)₂R^{b}, S(O)R^{b}, S(O)NR^{c}R^{d}, S(O)₂R^{b}, or S(O)₂NR^{c}R^{d}, or any subgroup thereof. In some embodiments, A² is halo, CN, NO₂, OR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, NR^{c}S(O)₂R^{b}, S(O)₂R^{b}, S(O)₂NR^{c}R^{d}, C₁₋₄ alkoxy, C₁₋₄haloalkoxy, amino, C₁₋₄ alkylamino, C₂₋₈ dialkylamino, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl or heterocycloalkyl, wherein each of the C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl or heterocycloalkyl is optionally substituted by 1, 2, 3, 4 or 5 halo, C₁₋₆ alkyl, C₂₋₆ alkenyl, C₂₋₆ alkynyl, C₁₋₄ haloalkyl, aryl, cycloalkyl, heteroaryl, heterocycloalkyl, CN, NO₂, OR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, NR^{c}S(O)₂R^{b}, S(O)R^{b}, S(O)₂R^{b}, or S(O)₂NR^{c}R^{d}.

In some embodiments, R^{Q} is halo, CN, NO₂, OR^{a}, SR^{a}, C(O)R^{b}, C(O)NR^{c}R^{d}, C(O)OR^{a}, OC(O)R^{b}, OC(O)NR^{c}R^{d}, S(O)R^{b}, S(O)NR^{c}R^{d}, S(O)₂R^{b}, S(O)₂NR^{c}R^{d}, C₁₋₄ alkoxy, C₁₋₄ haloalkoxy, C₁₋₆ alkyl, C₂₋₆ alkenyl, C₂₋₆ alkynyl, arylalkyl, cycloalkylalkyl, heteroarylalkyl or heterocycloalkylalkyl, or any subgroup thereof, wherein each of the C₁₋₆ alkyl, C₂₋₆ alkenyl, C₂₋₆ alkynyl, arylalkyl, cycloalkylalkyl, heteroarylalkyl or heterocycloalkylalkyl is optionally substituted by 1, 2, 3, 4 or 5 halo, C₁₋₆ alkyl, C₂₋₆ alkenyl, C₂₋₆ alkynyl, C₁₋₄ haloalkyl, aryl, cycloalkyl, heteroaryl, heterocycloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, NR^{c}S(O)R^{b}, NR^{c}S(O)₂R^{b}, S(O)R^{b}, S(O)NR^{c}R^{d}, S(O)₂R^{b}, or S(O)₂NR^{c}R^{d}, or any subgroup thereof.

In some embodiments, R^{a} and R^{a′} are each, independently, H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl, or any subgroup thereof, wherein the C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl is optionally substituted with OH, amino, halo, C₁₋₆ alkyl, C₁₋₆ haloalkyl, aryl, arylalkyl, heteroaryl, heteroarylalkyl, cycloalkyl or heterocycloalkyl, or any subgroup thereof.

In some embodiments, R^{b} and R^{b′} are each, independently, H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl, or any subgroup thereof, wherein the C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl is optionally substituted with OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl, or any subgroup thereof.

In some embodiments, R^{c} and R^{d} are each, independently, H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl, or any subgroup thereof, wherein the C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl is optionally substituted with OH, amino, halo, C₁₋₆ alkyl, C₁₋₆haloalkyl, C₁₋₆haloalkyl, aryl, arylalkyl, heteroaryl, heteroarylalkyl, cycloalkyl or heterocycloalkyl, or any subgroup thereof.

In some embodiments, R^{c} and R^{d} together with the N atom to which they are attached form a 4-, 5-, 6- or 7-membered heterocycloalkyl group, or any subgroup thereof.

In some embodiments, R^{c′} and R^{d′} are each, independently, H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl, or any subgroup thereof, wherein the C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl is optionally substituted with OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl, or any subgroup thereof.

In some embodiments, R^{c′} and R^{d′} together with the N atom to which they are attached form a 4-, 5-, 6- or 7-membered heterocycloalkyl group, or any subgroup thereof.

In some embodiments, R³ is C₁₋₁₀ alkyl, arylalkyl, heteroarylalkyl, cycloalkylalkyl or heterocycloalkylalkyl, wherein the C₁₋₁₀ alkyl, arylalkyl, heteroarylalkyl, cycloalkylalkyl or heterocycloalkylalkyl are each optionally substituted with 1, 2 or 3 A²; and A² is halo, CN, NO₂, OR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, NR^{c}S(O)₂R^{b}, S(O)₂R^{b}, S(O)₂NR^{c}R^{d}, C₁₋₄ alkoxy, C₁₋₄ haloalkoxy, amino, C₁₋₄ alkylamino, C₂₋₈ dialkylamino, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl or heterocycloalkyl, wherein each of the C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl or heterocycloalkyl is optionally substituted by 1, 2, 3, 4 or 5 halo, C₁₋₆ alkyl, C₂₋₆ alkenyl, C₂₋₆ alkynyl, C₁₋₄ haloalkyl, aryl, cycloalkyl, heteroaryl, heterocycloalkyl, CN, NO₂, OR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, NR^{c}S(O)₂R^{b}, S(O)R^{b}, S(O)₂R^{b}, or S(O)₂NR^{c}R^{d}.

Also provided herein are novel compounds of structural formula V:

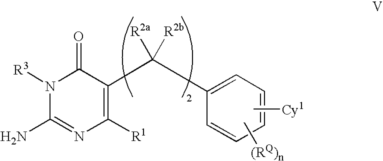

or a pharmaceutically acceptable salt, tautomer or in vivo-hydrolysable precursor thereof.

In some embodiments, R¹ is H, C₁₋₆ alkyl, aryl, heteroaryl, cycloalkyl, heterocycloalkyl, arylalkyl, heteroarylalkyl, cycloalkylalkyl or heterocycloalkylalkyl, or any subgroup thereof.

In some embodiments, R^{2a} and R^{2b} are each, independently, H or C₁₋₄ alkyl, or any subgroup thereof.

In some embodiments, R³ is C₁₋₁₀ alkyl, arylalkyl, heteroarylalkyl, cycloalkylalkyl or heterocycloalkylalkyl, or any subgroup thereof, wherein the C₁₋₁₀ alkyl, arylalkyl, heteroarylalkyl, cycloalkylalkyl or heterocycloalkylalkyl are each optionally substituted with 1, 2, 3, 4 or 5 halo, CN, NO₂, OR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, NR^{c}S(O)₂R^{b}, S(O)₂R^{b}, S(O)₂NR^{c}R^{d}, C₁₋₄ alkoxy, C₁₋₄ haloalkoxy, amino, C₁₋₄ alkylamino, C₂₋₈ dialkylamino, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl or heterocycloalkyl, or any subgroup thereof.

In some embodiments, R^{Q} is halo, CN, C₁₋₄ alkoxy, C₁₋₄ haloalkoxy, C₁₋₆ alkyl, C₂₋₆ alkenyl, C₂₋₆ alkynyl, arylalkyl, cycloalkylalkyl, heteroarylalkyl or heterocycloalkylalkyl, or any subgroup thereof.

In some embodiments, Cy¹ is aryl or heteroaryl, or any subgroup thereof, each optionally substituted by 1, 2, 3, 4 or 5 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl, or any subgroup thereof.

In some embodiments, n is 0 or 1.

Also provided herein are novel compounds of structural formula VI:

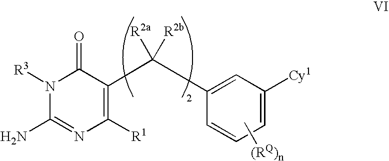

or a pharmaceutically acceptable salt, tautomer or in vivo-hydrolysable precursor thereof.

In some embodiments, R¹ is H, C₁₋₆ alkyl, aryl, heteroaryl, cycloalkyl, heterocycloalkyl, arylalkyl, heteroarylalkyl, cycloalkylalkyl or heterocycloalkylalkyl, or any subgroup thereof.

In some embodiments, R^{2a} and R^{2b} are each, independently, H or C₁₋₄ alkyl, or any subgroup thereof.

In some embodiments, R³ is C₁₋₁₀ alkyl, arylalkyl, heteroarylalkyl, cycloalkylalkyl or heterocycloalkylalkyl, or any subgroup thereof, wherein the C₁₋₁₀ alkyl, arylalkyl, heteroarylalkyl, cycloalkylalkyl or heterocycloalkylalkyl are each optionally substituted with 1, 2, 3, 4 or 5 halo, CN, NO₂, OR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, NR^{c}S(O)₂R^{b}, S(O)₂R^{b}, S(O)₂NR^{c}R^{d}, C₁₋₄ alkoxy, C₁₋₄haloalkoxy, amino, C₁₋₄ alkylamino, C₂₋₈ dialkylamino, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl or heterocycloalkyl, or any subgroup thereof.

In some embodiments, R^{Q} is halo, CN, C₁₋₄ alkoxy, C₁₋₄ haloalkoxy, C₁₋₆ alkyl, C₂₋₆ alkenyl, C₂₋₆ alkynyl, arylalkyl, cycloalkylalkyl, heteroarylalkyl or heterocycloalkylalkyl, or any subgroup thereof.

In some embodiments, Cy¹ is aryl or heteroaryl, each optionally substituted by 1, 2, 3, 4 or 5 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl, or any subgroup thereof. In some embodiments, Cy¹ is phenyl optionally substituted by 1, 2, 3, 4 or 5 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl.

In some embodiments, n is 0 or 1. In some embodiments, n is 0.

In some embodiments, n is 0; and Cy¹ is phenyl optionally substituted by 1, 2, 3, 4 or 5 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl.

Also provided herein are novel compounds of structural formula VII:

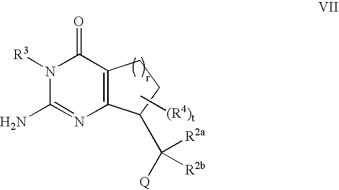

or a pharmaceutically acceptable salt, tautomer or in vivo-hydrolysable precursor thereof.

In some embodiments, R³ is H, C(O)R^{a}, C(O)OR^{b}, C(O)NR^{c}R^{d}, S(O)R^{a}, S(O)₂R^{a}, C₁₋₁₀ alkyl, C₂₋₁₀ alkenyl, C₂₋₁₀ alkynyl, cycloalkyl, heterocycloalkyl, arylalkyl, heteroarylalkyl, cycloalkylalkyl or heterocycloalkylalkyl, or any subgroup thereof, wherein the C₁₋₁₀ alkyl, C₂₋₁₀ alkenyl, C₂₋₁₀ alkynyl, cycloalkyl, heterocycloalkyl, arylalkyl, heteroarylalkyl, cycloalkylalkyl or heterocycloalkylalkyl are each optionally substituted with 1, 2, 3, 4 or 5 A², or any subgroup thereof. In some embodiments, R³ is C₁₋₁₀ alkyl, C₂₋₁₀ alkenyl, C₂₋₁₀ alkynyl, cycloalkyl, heterocycloalkyl, arylalkyl, heteroarylalkyl, cycloalkylalkyl or heterocycloalkylalkyl, wherein the C₁₋₁₀ alkyl, C₂₋₁₀ alkenyl, C₂₋₁₀ alkynyl, cycloalkyl, heterocycloalkyl, arylalkyl, heteroarylalkyl, cycloalkylalkyl or heterocycloalkylalkyl are each optionally substituted with 1, 2, 3, 4 or 5 A². In some embodiments, R³ is C₁₋₁₀ alkyl, arylalkyl, heteroarylalkyl, cycloalkylalkyl or heterocycloalkylalkyl, wherein the C₁₋₁₀ alkyl, arylalkyl, heteroarylalkyl, cycloalkylalkyl or heterocycloalkylalkyl are each optionally substituted with 1, 2, 3, 4 or 5 A². In some embodiments, R³ is C₁₋₁₀ alkyl, arylalkyl, heteroarylalkyl, cycloalkylalkyl or heterocycloalkylalkyl, wherein the C₁₋₁₀ alkyl, arylalkyl, heteroarylalkyl, cycloalkylalkyl or heterocycloalkylalkyl are each optionally substituted with 1, 2 or 3 A². In some embodiments, R³ is C₁₋₁₀ alkyl, arylalkyl, heteroarylalkyl, cycloalkylalkyl or heterocycloalkylalkyl. In some embodiments, R³ is C₁₋₁₀ alkyl.

In some embodiments, R⁴ is halo, CN, OR^{a}, SR^{a}, C(O)R^{b}, C(O)NR^{c}R^{d}, C(O)OR^{a}, OC(O)R^{b}, OC(O)NR^{c}R^{d}, S(O)R^{b}, S(O)NR^{c}R^{d}, S(O)₂R^{b}, S(O)₂NR^{c}R^{d}, C₁₋₆ alkyl, C₁₋₆ haloalkyl, aryl, heteroaryl, cycloalkyl, heterocycloalkyl, arylalkyl, heteroarylalkyl, cycloalkylalkyl or heterocycloalkylalkyl, or any subgroup thereof, wherein the C₁₋₆ alkyl, aryl, heteroaryl, cycloalkyl, heterocycloalkyl, arylalkyl, heteroarylalkyl, cycloalkylalkyl or heterocycloalkylalkyl is optionally substituted by 1, 2, 3, 4 or 5 A¹, or any subgroup thereof. In some embodiments, R⁴ is halo, C₁₋₆ alkyl, aryl, heteroaryl, cycloalkyl, heterocycloalkyl, arylalkyl, heteroarylalkyl, cycloalkylalkyl or heterocycloalkylalkyl, wherein the C₁₋₆ alkyl, aryl, heteroaryl, cycloalkyl, heterocycloalkyl, arylalkyl, heteroarylalkyl, cycloalkylalkyl or heterocycloalkylalkyl is optionally substituted by 1, 2 or 3 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl. In some embodiments, R⁴ is C₁₋₆ alkyl, aryl, heteroaryl, cycloalkyl, heterocycloalkyl, arylalkyl, heteroarylalkyl, cycloalkylalkyl or heterocycloalkylalkyl.

In some embodiments, R^{2a} and R^{2b} are each, independently, H, halo, C₁₋₄ alkyl, C₁₋₄ haloalkyl, C₂₋₆ alkenyl, C₂₋₆ alkynyl, aryl, cycloalkyl, heteroaryl, heterocycloalkyl, CN, NO₂, OR^{a′}, SR^{a′}, C(O)R^{b′}, C(O)NR^{c′}R^{d′}, C(O)OR^{a′}, OC(O)R^{b′}, OC(O)NR^{c′}R^{d′}, NR^{c′}R^{d′}, NR^{c′}C(O)R^{d′}, NR^{c′}C(O)OR^{a′}, NR^{c′}S(O)₂R^{b′}, S(O)R^{b′}, S(O)NR^{c′}R^{d′}, S(O)₂R^{b′}, or S(O)₂NR^{c′}R^{d′}, or any subgroup thereof. In some embodiments, R^{2a} and R^{2b} are each, independently, H, halo, C₁₋₄ alkyl, C₁₋₄ haloalkyl, C₂₋₆ alkenyl, C₂₋₆ alkynyl, aryl, cycloalkyl, heteroaryl, heterocycloalkyl, NO₂, OR^{a′}, SR^{a′}, OC(O)R^{b′}, OC(O)NR^{c′}R^{d′}, S(O)R^{b′}, S(O)NR^{c′}R^{d′}, S(O)₂R^{b′}, or S(O)₂NR^{c′}R^{d′}. In some embodiments, R^{2a} and R^{2b} are each, independently, H, halo, C₁₋₄ alkyl, C₁₋₄ haloalkyl, C₂₋₆ alkenyl, C₂₋₆ alkynyl, aryl, cycloalkyl, heteroaryl or heterocycloalkyl. In some embodiments, R^{2a} and R^{2b} are each, independently, H or C₁₋₄ alkyl. In some embodiments, R^{2a} and R^{2b} are both H.

In some embodiments, r is 0, 1, 2 or 3.

In some embodiments, t is 0, 1, 2, 3, 4 or 5.

In some embodiments, Q is aryl, cycloalkyl, heteroaryl or heterocycloalkyl, each optionally substituted by 1, 2, 3, 4 or 5 Cy¹ or R^{Q}, or any subgroup thereof. In some embodiments, Q is aryl or heteroaryl, each optionally substituted by 1, 2, 3, 4 or 5 Cy¹ or R^{Q}. In some embodiments, Q is aryl optionally substituted by 1, 2, 3, 4 or 5 Cy¹ or R^{Q}. In some embodiments, Q is aryl optionally substituted by 1, 2 or 3 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl or heterocycloalkylalkyl.

In some embodiments, Q is aryl substituted by Cy¹ and optionally substituted by 1, 2 or 3 R^{Q}.

In some embodiments, Q is phenyl wherein the phenyl is meta-substituted by Cy¹.

In some embodiments, Cy¹ is aryl, heteroaryl, cycloalkyl, or heterocycloalkyl, or any subgroup thereof, each optionally substituted with 1, 2, 3, 4 or 5 A³. In some embodiments, Cy¹ is aryl or heteroaryl, each optionally substituted by 1, 2, 3, 4 or 5 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl. In some embodiments, Cy¹ is aryl optionally substituted by 1, 2, 3, 4 or 5 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl.

In some embodiments, A¹, A², and A³ are each, independently, halo, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, NR^{c}S(O)R^{b}, NR^{c}S(O)₂R^{b}, S(O)R^{b}, S(O)NR^{c}R^{d}, S(O)₂R^{b}, S(O)₂NR^{c}R^{d}, C₁₋₄ alkoxy, C₁₋₄ haloalkoxy, amino, C₁₋₄ alkylamino, C₂₋₈ dialkylamino, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl or heterocycloalkyl, or any subgroup thereof, wherein each of the C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl or heterocycloalkyl is optionally substituted by 1, 2, 3, 4 or 5 halo, C₁₋₆ alkyl, C₂₋₆ alkenyl, C₂₋₆ alkynyl, C₁₋₄ haloalkyl, aryl, cycloalkyl, heteroaryl, heterocycloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, NR^{c}S(O)R^{b}, NR^{c}S(O)₂R^{b}, S(O)R^{b}, S(O)NR^{c}R^{d}, S(O)₂R^{b}, or S(O)₂NR^{c}R^{d}, or any subgroup thereof.

In some embodiments, A² is halo, CN, NO₂, OR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, NR^{c}S(O)₂R^{b}, S(O)₂R^{b}, S(O)₂NR^{c}R^{d}, C₁₋₄ alkoxy, C₁₋₄ haloalkoxy, amino, C₁₋₄ alkylamino, C₂₋₈ dialkylamino, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl or heterocycloalkyl, wherein each of the C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl or heterocycloalkyl is optionally substituted by 1, 2, 3, 4 or 5 halo, C₁₋₆ alkyl, C₂₋₆ alkenyl, C₂₋₆ alkynyl, C₁₋₄ haloalkyl, aryl, cycloalkyl, heteroaryl, heterocycloalkyl, CN, NO₂, OR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, NR^{c}S(O)₂R^{b}, S(O)R^{b}, S(O)₂R^{b}, or S(O)₂NR^{c}R^{d}.

In some embodiments, R^{Q} is halo, CN, NO₂, OR^{a}, SR^{a}, C(O)R^{b}, C(O)NR^{c}R^{d}, C(O)OR^{a}, OC(O)R^{b}, OC(O)NR^{c}R^{d}, S(O)R^{b}, S(O)NR^{c}R^{d}, S(O)₂R^{b}, S(O)₂NR^{c}R^{d}, C₁₋₄ alkoxy, C₁₋₄ haloalkoxy, C₁₋₆ alkyl, C₂₋₆ alkenyl, C₂₋₆ alkynyl, arylalkyl, cycloalkylalkyl, heteroarylalkyl or heterocycloalkylalkyl, or any subgroup thereof, wherein each of the C₁₋₆ alkyl, C₂₋₆ alkenyl, C₂₋₆ alkynyl, arylalkyl, cycloalkylalkyl, heteroarylalkyl or heterocycloalkylalkyl is optionally substituted by 1, 2, 3, 4 or 5 halo, C₁₋₆ alkyl, C₂₋₆ alkenyl, C₂₋₆ alkynyl, C₁₋₄ haloalkyl, aryl, cycloalkyl, heteroaryl, heterocycloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, NR^{c}S(O)R^{b}, NR^{c}S(O)₂R^{b}, S(O)R^{b}, S(O)NR^{c}R^{d}, S(O)₂R^{b}, or S(O)₂NR^{c}R^{d}, or any subgroup thereof.

In some embodiments, R^{a} and R^{a′} are each, independently, H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl, or any subgroup thereof, wherein the C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl is optionally substituted with OH, amino, halo, C₁₋₆ alkyl, C₁₋₆ haloalkyl, aryl, arylalkyl, heteroaryl, heteroarylalkyl, cycloalkyl or heterocycloalkyl, or any subgroup thereof.

In some embodiments, R^{b} and R^{b′} are each, independently, H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl, or any subgroup thereof, wherein the C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl is optionally substituted with OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl, or any subgroup thereof.

In some embodiments, R^{c} and R^{d} are each, independently, H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl, or any subgroup thereof, wherein the C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl is optionally substituted with OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl, or any subgroup thereof.

In some embodiments, R^{c} and R^{d} together with the N atom to which they are attached form a 4-, 5-, 6- or 7-membered heterocycloalkyl group.

In some embodiments, R^{c′} and R^{d′} are each, independently, H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl, or any subgroup thereof, wherein the C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl is optionally substituted with OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl, or any subgroup thereof.

In some embodiments, R^{c′} and R^{d′} together with the N atom to which they are attached form a 4-, 5-, 6- or 7-membered heterocycloalkyl group.

In some embodiments, Q is aryl substituted by Cy¹ and optionally substituted by 1, 2 or 3 R^{Q}; and Cy¹ is aryl or heteroaryl, each optionally substituted by 1, 2, 3, 4 or 5 substituents independently selected from halo, CN, OH, C₁₋₆ alkoxy, C₁₋₆haloalkoxy, C₁₋₆haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl.

In some embodiments, Q is phenyl wherein the phenyl is meta-substituted by Cy¹; and Cy¹ is aryl or heteroaryl, each optionally substituted by 1, 2, 3, 4 or 5 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl.

In some embodiments, Q is phenyl wherein the phenyl is meta-substituted by Cy¹; and Cy¹ is aryl optionally substituted by 1, 2, 3, 4 or 5 substituents independently selected from halo, CN, OH, C₁₋₆ alkoxy, C₁₋₆haloalkoxy, C₁₋₆haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl.

In some embodiments, R³ is C₁₋₁₀ alkyl, arylalkyl, heteroarylalkyl, cycloalkylalkyl or heterocycloalkylalkyl, wherein the C₁₋₁₀ alkyl, arylalkyl, heteroarylalkyl, cycloalkylalkyl or heterocycloalkylalkyl are each optionally substituted with 1, 2 or 3 A²; and A² is halo, CN, NO₂, OR^{a}, C(O)R^{b}, C(O)NR^{c}R^{d}, C(O)OR^{a}, OC(O)R^{b}, OC(O)NR^{c}R^{d}, NR^{c}R^{d}. NR^{c}C(O)R^{d}, NR^{c}C(O)OR^{a}, NR^{c}S(O)₂R^{b}, S(O)₂R^{b}, S(O)₂NR^{c}R^{d}, C₁₋₄ alkoxy, C₁₋₄ haloalkoxy, amino, C₁₋₄ alkylamino, C₂₋₈ dialkylamino, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl or heterocycloalkyl, wherein each of the C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl or heterocycloalkyl is optionally substituted by 1, 2, 3, 4 or 5 halo, C₁₋₆ alkyl, C₂₋₆ alkenyl, C₂₋₆ alkynyl, C₁₋₄ haloalkyl, aryl, cycloalkyl, heteroaryl, heterocycloalkyl, CN, NO₂, OR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, NR^{c}S(O)₂R^{b}, S(O)R^{b}, S(O)₂R^{b}, or S(O)₂NR^{c}R^{d}.

Also provided herein are novel compounds of structural formula VIII:

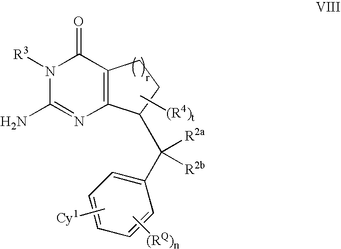

or a pharmaceutically acceptable salt, tautomer or in vivo-hydrolysable precursor thereof.

In some embodiments, R⁴ is C₁₋₆ alkyl, aryl, heteroaryl, cycloalkyl, heterocycloalkyl, arylalkyl, heteroarylalkyl, cycloalkylalkyl or heterocycloalkylalkyl, or any subgroup thereof.

In some embodiments, R^{2a} and R^{2b} are each, independently, H or C₁₋₄ alkyl, or any subgroup thereof.

In some embodiments, R³ is C₁₋₁₀ alkyl, arylalkyl, heteroarylalkyl, cycloalkylalkyl or heterocycloalkylalkyl, or any subgroup thereof, wherein the C₁₋₁₀ alkyl, arylalkyl, heteroarylalkyl, cycloalkylalkyl or heterocycloalkylalkyl are each optionally substituted with 1, 2, 3, 4 or 5 halo, CN, NO₂, OR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, NR^{c}S(O)₂R^{b}, S(O)₂R^{b}, S(O)₂NR^{c}R^{d}, C₁₋₄ alkoxy, C₁₋₄ haloalkoxy, amino, C₁₋₄ alkylamino, C₂₋₈ dialkylamino, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl or heterocycloalkyl, or any subgroup thereof.

In some embodiments, R^{Q} is halo, CN, C₁₋₄ alkoxy, C₁₋₄ haloalkoxy, C₁₋₆ alkyl, C₂₋₆ alkenyl, C₂₋₆ alkynyl, arylalkyl, cycloalkylalkyl, heteroarylalkyl or heterocycloalkylalkyl, or any subgroup thereof.

In some embodiments, Cy¹ is aryl or heteroaryl, each optionally substituted by 1, 2, 3, 4 or 5 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl, or any subgroup thereof.

In some embodiments, n is 0 or 1.

In some embodiments, r is 1 or 2.

In some embodiments, t is 0, 1, 2 or 3.

Also provided herein are novel compounds of structural formula IX:

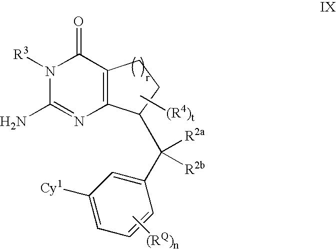

or a pharmaceutically acceptable salt, tautomer or in vivo-hydrolysable precursor thereof.

In some embodiments, R⁴ is C₁₋₆ alkyl, aryl, heteroaryl, cycloalkyl, heterocycloalkyl, arylalkyl, heteroarylalkyl, cycloalkylalkyl or heterocycloalkylalkyl, or any subgroup thereof.

In some embodiments, R^{2a} and R^{2b} are each, independently, H or C₁₋₄ alkyl, or any subgroup thereof.

In some embodiments, R³ is C₁₋₁₀ alkyl, arylalkyl, heteroarylalkyl, cycloalkylalkyl or heterocycloalkylalkyl, or any subgroup thereof, wherein the C₁₋₁₀ alkyl, arylalkyl, heteroarylalkyl, cycloalkylalkyl or heterocycloalkylalkyl are each optionally substituted with 1, 2, 3, 4 or 5 halo, CN, NO₂, OR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, NR^{c}S(O)₂R^{b}, S(O)₂R^{b}, S(O)₂NR^{c}R^{d}, C₁₋₄ alkoxy, C₁₋₄haloalkoxy, amino, C₁₋₄ alkylamino, C₂₋₈ dialkylamino, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl or heterocycloalkyl, or any subgroup thereof.

In some embodiments, R^{Q} is halo, CN, C₁₋₄ alkoxy, C₁₋₄ haloalkoxy, C₁₋₆ alkyl, C₂₋₆ alkenyl, C₂₋₆ alkynyl, arylalkyl, cycloalkylalkyl, heteroarylalkyl or heterocycloalkylalkyl, or any subgroup thereof.

In some embodiments, Cy¹ is aryl or heteroaryl, each optionally substituted by 1, 2, 3, 4 or 5 substituents independently selected from halo, CN, OH, C₁₋₆ alkoxy, C₁₋₆haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl, or any subgroup thereof. In some embodiments, Cy¹ is phenyl optionally substituted by 1, 2, 3, 4 or 5 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl.

In some embodiments, n is 0 or 1. In some embodiments, n is 0.

In some embodiments, r is 1 or 2.

In some embodiments, t is 0, 1, 2 or 3.

In some embodiments, n is 0; and Cy¹ is phenyl optionally substituted by 1, 2, 3, 4 or 5 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl.

Also provided herein are novel compounds of structural formula X:

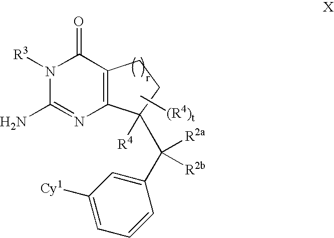

or a pharmaceutically acceptable salt, tautomer or in vivo-hydrolysable precursor thereof.

In some embodiments, R⁴ is C₁₋₆ alkyl, aryl, heteroaryl, cycloalkyl, heterocycloalkyl, arylalkyl, heteroarylalkyl, cycloalkylalkyl or heterocycloalkylalkyl, or any subgroup thereof.

In some embodiments, R^{2a} and R^{2b} are each, independently, H or C₁₋₄ alkyl, or any subgroup thereof.

In some embodiments, R³ is C₁₋₁₀ alkyl, arylalkyl, heteroarylalkyl, cycloalkylalkyl or heterocycloalkylalkyl, or any subgroup thereof, wherein the C₁₋₁₀ alkyl, arylalkyl, heteroarylalkyl, cycloalkylalkyl or heterocycloalkylalkyl are each optionally substituted with 1, 2, 3, 4 or 5 halo, CN, NO₂, OR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, NR^{c}S(O)₂R^{b}, S(O)₂R^{b}, S(O)₂NR^{c}R^{d}, C₁₋₄ alkoxy, C₁₋₄ haloalkoxy, amino, C₁₋₄ alkylamino, C₂₋₈ dialkylamino, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl or heterocycloalkyl, or any subgroup thereof.

In some embodiments, Cy¹ is aryl or heteroaryl, each optionally substituted by 1, 2, 3, 4 or 5 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl, or any subgroup thereof. In some embodiments, Cy¹ is phenyl optionally substituted by 1, 2, 3, 4 or 5 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl.

In some embodiments, r is 1 or 2.

In some embodiments, t is 0, 1 or 2.

Also provided herein are novel compounds of structural formula XI:

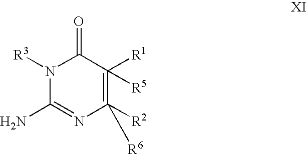

or a pharmaceutically acceptable salt, tautomer or in vivo-hydrolysable precursor thereof.

In some embodiments, R¹ is C₁₋₆ alkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl, each optionally substituted by 1, 2, 3, 4 or 5 A¹, or any subgroup thereof. In some embodiments, R¹ is C₁₋₆ alkyl, cycloalkyl, heterocycloalkyl, arylalkyl, heteroarylalkyl, cycloalkylalkyl or heterocycloalkylalkyl, each optionally substituted by 1, 2 or 3 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl. In some embodiments, R¹ is C₁₋₆ alkyl, cycloalkyl, heterocycloalkyl, arylalkyl, heteroarylalkyl, cycloalkylalkyl or heterocycloalkylalkyl.

In some embodiments, R² is —(CR^{2a}R^{2b})ₘ-Q.

In some embodiments, R³ is H, C(O)R^{a}, C(O)OR^{b}, C(O)NR^{c}R^{d}, S(O)R^{a}, S(O)₂R^{a}, C₁₋₁₀ alkyl, C₂₋₁₀ alkenyl, C₂₋₁₀ alkynyl, cycloalkyl, heterocycloalkyl, arylalkyl, heteroarylalkyl, cycloalkylalkyl or heterocycloalkylalkyl, or any subgroup thereof, wherein the C₁₋₁₀ alkyl, C₂₋₁₀ alkenyl, C₂₋₁₀ alkynyl, cycloalkyl, heterocycloalkyl, arylalkyl, heteroarylalkyl, cycloalkylalkyl or heterocycloalkylalkyl are each optionally substituted with 1, 2, 3, 4 or 5 A², or any subgroup thereof. In some embodiments, R³ is H, C₁₋₁₀ alkyl, C₂₋₁₀ alkenyl, C₂₋₁₀-alkynyl, cycloalkyl, heterocycloalkyl, arylalkyl, heteroarylalkyl, cycloalkylalkyl or heterocycloalkylalkyl, wherein the C₁₋₁₀ alkyl, C₂₋₁₀ alkenyl, C₂₋₁₀ alkynyl, cycloalkyl, heterocycloalkyl, arylalkyl, heteroarylalkyl, cycloalkylalkyl or heterocycloalkylalkyl are each optionally substituted with 1, 2, 3, 4 or 5 A². In some embodiments, R³ is H, C₁₋₁₀ alkyl, arylalkyl, heteroarylalkyl, cycloalkylalkyl or heterocycloalkylalkyl, wherein the C₁₋₁₀ alkyl, arylalkyl, heteroarylalkyl, cycloalkylalkyl or heterocycloalkylalkyl are each optionally substituted with 1, 2, 3, 4 or 5 A². In some embodiments, R³ is H, C₁₋₁₀ alkyl, arylalkyl, heteroarylalkyl, cycloalkylalkyl or heterocycloalkylalkyl, wherein each of the C₁₋₁₀ alkyl, arylalkyl, heteroarylalkyl, cycloalkylalkyl or heterocycloalkylalkyl are each optionally substituted with 1, 2 or 3 A². In some embodiments, R³ is C₁₋₁₀ alkyl, arylalkyl, heteroarylalkyl, cycloalkylalkyl or heterocycloalkylalkyl, wherein the C₁₋₁₀ alkyl, arylalkyl, heteroarylalkyl, cycloalkylalkyl or heterocycloalkylalkyl are each optionally substituted with 1, 2, 3, 4 or 5 halo, CN, NO₂, OR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, NR^{c}S(O)₂R^{b}, S(O)₂R^{b}, S(O)₂NR^{c}R^{d}, C₁₋₄ alkoxy, C₁₋₄ haloalkoxy, amino, C₁₋₄ alkylamino, C₂₋₈ dialkylamino, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl or heterocycloalkyl.

In some embodiments, R⁵ is H, C₁₋₆ alkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl, or any subgroup thereof, wherein each of the C₁₋₆ alkyl, aryl, heteroaryl, cycloalkyl, heterocycloalkyl, arylalkyl, heteroarylalkyl, cycloalkylalkyl or heterocycloalkylalkyl is optionally substituted by 1, 2, 3, 4 or 5 A⁴, or any subgroup thereof. In some embodiments, R⁵ is H.

In some embodiments, R⁶ is H, C₁₋₆ alkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl, or any subgroup thereof, wherein each of the C₁₋₆ alkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl is optionally substituted by 1, 2, 3, 4 or 5 A⁵, or any subgroup thereof. In some embodiments, R⁶ is C₁₋₁₀ alkyl.

In some embodiments, R^{2a} and R^{2b} are each, independently, H, halo, C₁₋₄ alkyl, C₁₋₄ haloalkyl, C₂₋₆ alkenyl, C₂₋₆ alkynyl, aryl, cycloalkyl, heteroaryl, heterocycloalkyl, CN, NO₂, OR^{a′}, SR^{a′}, C(O)R^{b′}, C(O)NR^{c′}R^{d′}, C(O)OR^{a′}, OC(O)R^{b′}, OC(O)NR^{c′}R^{d′}, NR^{c′}R^{d′}, NR^{c′}C(O)R^{d′}, NR^{c′}C(O)OR^{a′}, NR^{c′}S(O)₂R^{b′}, S(O)R^{b′}, S(O)NR^{c′}R^{d′}, S(O)₂R^{b′}, or S(O)₂NR^{c′}R^{d′}, or any subgroup thereof. In some embodiments, R^{2a} and R^{2b} are each, independently, H, halo, C₁₋₄ alkyl, C₁₋₄ haloalkyl, C₂₋₆ alkenyl, C₂₋₆ alkynyl, aryl, cycloalkyl, heteroaryl or heterocycloalkyl. In some embodiments, R^{2a} and R^{2b} are each, independently, H or C₁₋₄ alkyl. In some embodiments, R^{2a} and R^{2b} are both H. In some embodiments, R^{2a} and R^{2b} are each, independently, H or C₁₋₄ alkyl.

In some embodiments, m is 0, 1, 2, 3 or 4. In some embodiments, m is 0. In some embodiments, m is 0, 1 or 2.

In some embodiments, Q is aryl, cycloalkyl, heteroaryl or heterocycloalkyl, or any subgroup thereof, each optionally substituted by 1, 2, 3, 4 or 5 Cy¹ or R^{Q}. In some embodiments, Q is aryl or heteroaryl, each optionally substituted by 1, 2, 3, 4 or 5 Cy¹ or R^{Q}. In some embodiments, Q is aryl optionally substituted by 1, 2, 3, 4 or 5 Cy¹ or R^{Q}. In some embodiments, Q is aryl optionally substituted by 1, 2 or 3 R^{Q}. In some embodiments, Q is aryl substituted by Cy¹ and optionally substituted by 1, 2 or 3 R^{Q}. In some embodiments, Q is aryl substituted by Cy¹ and optionally substituted by 1, 2 or 3 R^{Q}; and Cy¹ is aryl or heteroaryl, each optionally substituted by 1, 2, 3, 4 or 5 substituents independently selected from halo, CN, OH, C₁₋₆ alkoxy, C₁₋₆haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl. In some embodiments, Q is phenyl wherein the phenyl is meta-substituted by Cy¹.

In some embodiments, Cy¹ is aryl, heteroaryl, cycloalkyl, or heterocycloalkyl, or any subgroup thereof, each optionally substituted with 1, 2, 3, 4 or 5 A³. In some embodiments, Cy¹ is aryl or heteroaryl, each optionally substituted by 1, 2, 3, 4 or 5 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl. In some embodiments, Cy¹ is aryl optionally substituted by 1, 2, 3, 4 or 5 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl.

In some embodiments, A¹, A², A³, A⁴, and A⁵ are each, independently, halo, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, NR^{c}S(O)R^{b}, NR^{c}S(O)₂R^{b}, S(O)R^{b}, S(O)NR^{c}R^{d}, S(O)₂R^{b}, S(O)₂NR^{c}R^{d}, C₁₋₄ alkoxy, C₁₋₄haloalkoxy, amino, C₁₋₄ alkylamino, C₂₋₈ dialkylamino, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl or heterocycloalkyl, wherein each of the C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl or heterocycloalkyl is optionally substituted by 1, 2, 3, 4 or 5 halo, C₁₋₆ alkyl, C₂₋₆ alkenyl, C₂₋₆ alkynyl, C₁₋₄ haloalkyl, aryl, cycloalkyl, heteroaryl, heterocycloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, NR^{c}S(O)R^{b}, NR^{c}S(O)₂R^{b}, S(O)R^{b}, S(O)NR^{c}R^{d}, S(O)₂R^{b}, or S(O)₂NR^{c}R^{d}, or any subgroup thereof. In some embodiments, A² is halo, CN, NO₂, OR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, NR^{c}S(O)₂R^{b}, S(O)₂R^{b}, S(O)₂NR^{c}R^{d}, C₁₋₄ alkoxy, C₁₋₄ haloalkoxy, amino, C₁₋₄ alkylamino, C₂₋₈ dialkylamino, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl or heterocycloalkyl, wherein each of the C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl or heterocycloalkyl is optionally substituted by 1, 2, 3, 4 or 5 halo, C₁₋₆ alkyl, C₂₋₆ alkenyl, C₂₋₆ alkynyl, C₁₋₄ haloalkyl, aryl, cycloalkyl, heteroaryl, heterocycloalkyl, CN, NO₂, OR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, NR^{c}S(O)₂R^{b}, S(O)R^{b}, S(O)₂R^{b}, or S(O)₂NR^{c}R^{d}.

In some embodiments, R^{Q} is halo, CN, NO₂, OR^{a}, SR^{a}, C(O)R^{b}, C(O)NR^{c}R^{d}, C(O)OR^{a}, OC(O)R^{b}, OC(O)NR^{c}R^{d}, S(O)R^{b}, S(O)NR^{c}R^{d}, S(O)₂R^{b}, S(O)₂NR^{c}R^{d}, C₁₋₄ alkoxy, C₁₋₄ haloalkoxy, C₁₋₆ alkyl, C₂₋₆ alkenyl, C₂₋₆ alkynyl, arylalkyl, cycloalkylalkyl, heteroarylalkyl or heterocycloalkylalkyl, or any subgroup thereof, wherein each of the C₁₋₆ alkyl, C₂₋₆ alkenyl, C₂₋₆ alkynyl, arylalkyl, cycloalkylalkyl, heteroarylalkyl or heterocycloalkylalkyl is optionally substituted by 1, 2, 3, 4 or 5 halo, C₁₋₆ alkyl, C₂₋₆ alkenyl, C₂₋₆ alkynyl, C₁₋₄ haloalkyl, aryl, cycloalkyl, heteroaryl, heterocycloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, NR^{c}S(O)R^{b}, NR^{c}S(O)₂R^{b}, S(O)R^{b}, S(O)NR^{c}R^{d}, S(O)₂R^{b}, or S(O)₂NR^{c}R^{d}, or any subgroup thereof. In some embodiments, R^{Q} is halo, CN, OH, C₁₋₆ alkoxy, C₁₋₆haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl or heterocycloalkylalkyl.

In some embodiments, R^{a} and R^{a′} are each, independently, H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl, or any subgroup thereof, wherein the C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl is optionally substituted with OH, amino, halo, C₁₋₆ alkyl, C₁₋₆ haloalkyl, aryl, arylalkyl, heteroaryl, heteroarylalkyl, cycloalkyl or heterocycloalkyl, or any subgroup thereof.

In some embodiments, R^{b} and R^{b′} are each, independently, H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl, or any subgroup thereof, wherein the C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl is optionally substituted with OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl, or any subgroup thereof.

In some embodiments, R^{c} and R^{d} are each, independently, H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl, or any subgroup thereof, wherein the C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl is optionally substituted with OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl, or any subgroup thereof.

In some embodiments, R^{c} and R^{d} together with the N atom to which they are attached form a 4-, 5-, 6- or 7-membered heterocycloalkyl group.

In some embodiments, R^{c′} and R^{d′} are each, independently, H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl, or any subgroup thereof, wherein the C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl is optionally substituted with OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl, or any subgroup thereof.

In some embodiments, R^{c′} and R^{d′} together with the N atom to which they are attached form a 4-, 5-, 6- or 7-membered heterocycloalkyl group.

In some embodiments, Q is phenyl wherein the phenyl is meta-substituted by Cy¹; and Cy¹ is aryl or heteroaryl, each optionally substituted by 1, 2, 3, 4 or 5 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl. In some embodiments, Q is phenyl optionally substituted by 1, 2 or 3 halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, or heterocycloalkylalkyl; m is 0, 1 or 2.

In some embodiments, Q is phenyl wherein the phenyl is meta-substituted by Cy¹; and Cy¹ is aryl optionally substituted by 1, 2, 3, 4 or 5 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl.

In some embodiments, R³ is H, C₁₋₁₀ alkyl, arylalkyl, heteroarylalkyl, cycloalkylalkyl or heterocycloalkylalkyl, wherein each of the C₁₋₁₀ alkyl, arylalkyl, heteroarylalkyl, cycloalkylalkyl or heterocycloalkylalkyl are each optionally substituted with 1, 2 or 3 A²; and A² is halo, CN, NO₂, OR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, NR^{c}S(O)₂R^{b}, S(O)₂R^{b}, S(O)₂NR^{c}R^{d}, C₁₋₄ alkoxy, C₁₋₄ haloalkoxy, amino, C₁₋₄ alkylamino, C₂₋₈ dialkylamino, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl or heterocycloalkyl, wherein each of the C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl or heterocycloalkyl is optionally substituted by 1, 2, 3, 4 or 5 halo, C₁₋₆ alkyl, C₂₋₆ alkenyl, C₂₋₆ alkynyl, C₁₋₄ haloalkyl, aryl, cycloalkyl, heteroaryl, heterocycloalkyl, CN, NO₂, OR^{a}, C(O)R^{b}, C(O)NR^{c}R^{d}, C(O)OR^{a}, OC(O)R^{b}, OC(O)NR^{c}R^{d}, N d, NR^{c}C(O)R^{d}, NR^{c}C(O)OR^{a}, NR^{c}S(O)₂R^{b}, S(O)R^{b}, S(O)₂R^{b}, or S(O)₂NR^{c}R^{d}. In some embodiments, R³ is H, C₁₋₁₀ alkyl, arylalkyl, heteroarylalkyl, cycloalkylalkyl or heterocycloalkylalkyl. In some embodiments, R³ is H or C₁₋₁₀ alkyl.

In some embodiments, R¹ is C₁₋₆ alkyl, cycloalkyl, heterocycloalkyl, arylalkyl, heteroarylalkyl, cycloalkylalkyl or heterocycloalkylalkyl; R^{2a} and R^{2b} are each, independently, H or C₁₋₄ alkyl; Q is aryl optionally substituted by 1, 2 or 3 R^{Q}; m is 0, 1 or 2; R³ is H, C₁₋₁₀ alkyl, arylalkyl, heteroarylalkyl, cycloalkylalkyl or heterocycloalkylalkyl, wherein the C₁₋₁₀ alkyl, arylalkyl, heteroarylalkyl, cycloalkylalkyl or heterocycloalkylalkyl are each optionally substituted with 1, 2, 3, 4 or 5 A²; R⁵ is H; and R⁶ is C₁₋₁₀ alkyl.

In some embodiments, R¹ is C₁₋₆ alkyl, cycloalkyl, heterocycloalkyl, arylalkyl, heteroarylalkyl, cycloalkylalkyl or heterocycloalkylalkyl; R^{2a} and R^{2b} are each, independently, H or C₁₋₄ alkyl; Q is phenyl optionally substituted by 1, 2 or 3 halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, or heterocycloalkylalkyl; m is 0, 1 or 2; R³ is C₁₋₁₀ alkyl, arylalkyl, heteroaryalkyl, cycloalkylalkyl or heterocycloalkylalkyl, wherein the C₁₋₁₀ alkyl, arylalkyl, heteroarylalkyl, cycloalkylalkyl or heterocycloalkylalkyl are each optionally substituted with 1, 2, 3, 4 or 5 halo, CN, NO₂, OR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, NR^{c}S(O)₂R^{b}, S(O)₂R^{b}, S(O)₂NR^{c}R^{d}, C₁₋₄alkoxy, C₁₋₄haloalkoxy, amino, C₁₋₄ alkylamino, C₂₋₈ dialkylamino, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl or heterocycloalkyl; R⁵ is H; and R⁶ is C₁₋₁₀ alkyl.

Also provided herein are novel compounds of structural formula XII:

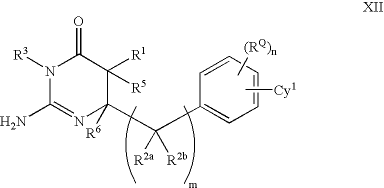

or a pharmaceutically acceptable salt, tautomer or in vivo-hydrolysable precursor thereof.

In some embodiments, R¹ is C₁₋₆ alkyl, cycloalkyl, heterocycloalkyl, arylalkyl, heteroarylalkyl, cycloalkylalkyl or heterocycloalkylalkyl, or any subgroup thereof.

In some embodiments, R^{2a} and R^{2b} are each, independently, H or C₁₋₄ alkyl.

In some embodiments, R³ is C₁₋₁₀ alkyl, arylalkyl, heteroarylalkyl, cycloalkylalkyl or heterocycloalkylalkyl, or any subgroup thereof, wherein the C₁₋₁₀ alkyl, arylalkyl, heteroarylalkyl, cycloalkylalkyl or heterocycloalkylalkyl are each optionally substituted with 1, 2, 3, 4 or 5 halo, CN, NO₂, OR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, NR^{c}S(O)₂R^{b}, S(O)₂R^{b}, S(O)₂NR^{c}R^{d}, C₁₋₄ alkoxy, C₁₋₄ haloalkoxy, amino, C₁₋₄ alkylamino, C₂₋₈ dialkylamino, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl or heterocycloalkyl, or any subgroup thereof.

In some embodiments, R⁵ is H.

In some embodiments, R⁶ is C₁₋₁₀ alkyl.

In some embodiments, R^{Q} is halo, CN, C₁₋₄ alkoxy, C₁₋₄ haloalkoxy, C₁₋₆ alkyl, C₂₋₆ alkenyl, C₂₋₆ alkynyl, arylalkyl, cycloalkylalkyl, heteroarylalkyl or heterocycloalkylalkyl, or any subgroup thereof.

In some embodiments, Cy¹ is aryl or heteroaryl, each optionally substituted by 1, 2, 3, 4 or 5 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl, or any subgroup thereof.

In some embodiments, m is 0, 1, or 2.

In some embodiments, n is 0 or 1.

Also provided herein are novel compounds of structural formula XIII:

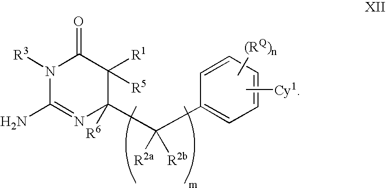

or a pharmaceutically acceptable salt, tautomer or in vivo-hydrolysable precursor thereof.

In some embodiments, R¹ is C₁₋₆ alkyl, cycloalkyl, heterocycloalkyl, arylalkyl, heteroarylalkyl, cycloalkylalkyl or heterocycloalkylalkyl, or any subgroup thereof.

In some embodiments, R^{2a} and R^{2b} are each, independently, H or C₁₋₄ alkyl.

In some embodiments, R³ is C₁₋₁₀ alkyl, arylalkyl, heteroarylalkyl, cycloalkylalkyl or heterocycloalkylalkyl, or any subgroup thereof, wherein the C₁₋₁₀ alkyl, arylalkyl, heteroarylalkyl, cycloalkylalkyl or heterocycloalkylalkyl are each optionally substituted with 1, 2, 3, 4 or 5 halo, CN, NO₂, OR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, NR^{c}S(O)₂R^{b}, S(O)₂R^{b}, S(O)₂NR^{c}R^{d}, C₁₋₄ alkoxy, C₁₋₄ haloalkoxy, amino, C₁₋₄ alkylamino, C₂₋₈ dialkylamino, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl or heterocycloalkyl, or any subgroup thereof.

In some embodiments, R⁵ is H.

In some embodiments, R⁶ is C₁₋₁₀ alkyl.

In some embodiments, R^{Q} is halo, CN, C₁₋₄ alkoxy, C₁₋₄ haloalkoxy, C₁₋₆ alkyl, C₂₋₆ alkenyl, C₂₋₆ alkynyl, arylalkyl, cycloalkylalkyl, heteroarylalkyl or heterocycloalkylalkyl, or any subgroup thereof.

In some embodiments, Cy¹ is aryl or heteroaryl, each optionally substituted by 1, 2, 3, 4 or 5 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl, or any subgroup thereof.

In some embodiments, m is 0, 1, or 2.

In some embodiments, n is 0 or 1.

Compounds of the present invention also include pharmaceutically acceptable salts, tautomers and in vivo-hydrolysable precursors of the compounds of any of the formulas described herein. Compounds of the invention further include hydrates and solvates.

The compounds of the invention include, for example:
2-amino-6-(3-bromo-4-chlorophenyl)-5,6-dimethyl-5,6-dihydropyrimidin-4(3H)-one trifluoroacetate;
2-amino-6-[2-(3′-methoxybiphenyl-3-yl)ethyl]-3,5-dimethylpyrimidin-4(3H)-one trifluoroacetate;
2-amino-3,5-dimethyl-6-(2-phenylethyl)pyrimidin-4(3H)-one trifluoroacetate;
2-amino-6-{2-[3-(2-furyl)phenyl]ethyl}-3,5-dimethylpyrimidin-4(3H)-one trifluoroacetate;
2-amino-6-[2-(3-bromophenyl)ethyl]-3,5-dimethylpyrimidin-4(3H)-one trifluoroacetate;
2-amino-6-[2-(3-bromophenyl)ethyl]-5-methylpyrimidin-4(3H)-one;
2-amino-5-benzyl-6-[2-(3′-methoxybiphenyl-3-yl)ethyl]-3-methylpyrimidin-4(3H)-one trifluoroacetate;
2-amino-5-benzyl-6-[2-(3-bromophenyl)ethyl]-3-methylpyrimidin-4(3H)-one trifluoroacetate;
2-amino-3-methyl-5-phenyl-6-(2-phenylethyl)pyrimidin-4(3H)-one trifluoroacetate;
2-amino-5-bromo-3-methyl-6-(2-phenylethyl)pyrimidin-4(3H)-one;
2-amino-3-methyl-6-(2-phenylethyl)pyrimidin-4(3H)-one;
2-amino-6-(2-phenylethyl)pyrimidin-4(3H)-one;
2-amino-6-[2-(3-bromophenyl)ethyl]pyrimidin-4(3H)-one;
2-amino-8-[(3′-methoxybiphenyl-3-yl)methyl]-3-methyl-5,6,7,8-tetrahydroquinazolin-4(3H)-one trifluoroacetate;
2-amino-8-(3-bromobenzyl)-3-methyl-5,6,7,8-tetrahydroquinazolin-4(3H)-one;
2-amino-8-(3-bromobenzyl)-5,6,7,8-tetrahydroquinazolin-4(3H)-one trifluoroacetate;
2-amino-8-[(3′-methoxybiphenyl-3-yl)methyl]-3,8-dimethyl-5,6,7,8-tetrahydroquinazolin-4(3H)-one trifluoroacetate;
2-amino-8-(3-bromobenzyl)-3,8-dimethyl-5,6,7,8-tetrahydroquinazolin-4(3H)-one;
2-amino-8-(3-bromobenzyl)-8-methyl-5,6,7,8-tetrahydroquinazolin-4(3H)-one;
2-amino-3-methyl-5-(2-phenylethyl)pyrimidin-4(3H)-one trifluoroacetate;
2-amino-1-methyl-5-(2-phenylethyl)pyrimidin-4(3H)-one trifluoroacetate;
2-amino-5-(2-phenylethyl)pyrimidin-4(3H)-one trifluoroacetate;
2-amino-5-[2-(3′-methoxybiphenyl-3-yl)ethyl]-3-methylpyrimidin-4(3H)-one trifluoroacetate;
2-amino-5-[2-(3′-methoxybiphenyl-3-yl)ethyl]pyrimidin-4(3H)-one trifluoroacetate;
   or a pharmaceutically acceptable salt, alternative salt, tautomer, or in vivo-hydrolysable precursor thereof.

Compounds of the invention can be used as medicaments. In some embodiments, the present invention provides compounds of any of the formulas described herein, or pharmaceutically acceptable salts, tautomers or in vivo-hydrolysable precursors thereof, for use as medicaments. In some embodiments, the present invention provides compounds described herein for use as medicaments for treating or preventing an Aβ-related pathology. In some further embodiments, the Aβ-related pathology is Downs syndrome, a β-amyloid angiopathy, cerebral amyloid angiopathy, hereditary cerebral hemorrhage, a disorder associated with cognitive impairment, MCI (“mild cognitive impairment”), Alzheimer Disease, memory loss, attention deficit symptoms associated with Alzheimer disease, neurodegeneration associated with Alzheimer disease, dementia of mixed vascular origin, dementia of degenerative origin, pre-senile dementia, senile dementia, dementia associated with Parkinson's disease, progressive supranuclear palsy or cortical basal degeneration.

In some embodiments, the present invention provides compounds of any of the formulas described herein, or pharmaceutically acceptable salts, tautomers or in vivo-hydrolysable precursors thereof, in the manufacture of a medicament for the treatment or prophylaxis of Aβ-related pathologies. In some further embodiments, the Aβ-related pathologies include such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration.

In some embodiments, the present invention provides a method of inhibiting activity of BACE comprising contacting the BACE with a compound of the present invention. BACE is thought to represent the major β-secretase activity, and is considered to be the rate-limiting step in the production of amyloid-β-protein (Aβ). Thus, inhibiting BACE through inhibitors such as the compounds provided herein would be useful to inhibit the deposition of Aβ and portions thereof. Because the deposition of Aβ and portions thereof is linked to diseases such as Alzheimer Disease, BACE is an important candidate for the development of drugs as a treatment and/or prophylaxis of Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration.

In some embodiments, the present invention provides a method for the treatment of Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration, comprising administering to a mammal (including human) a therapeutically effective amount of a compound of any of the formulas described herein, or a pharmaceutically acceptable salt, tautomer or in vivo-hydrolysable precursor thereof.

In some embodiments, the present invention provides a method for the prophylaxis of Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration comprising administering to a mammal (including human) a therapeutically effective amount of a compound of any of the formulas described herein or a pharmaceutically acceptable salt, tautomer or in vivo-hydrolysable precursors.

In some embodiments, the present invention provides a method of treating or preventing Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration by administering to a mammal (including human) a compound of any of the formulas described herein or a pharmaceutically acceptable salt, tautomer or in vivo-hydrolysable precursors and a cognitive and/or memory enhancing agent.

In some embodiments, the present invention provides a method of treating or preventing Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration by administering to a mammal (including human) a compound of any of the formulas described herein or a pharmaceutically acceptable salt, tautomer or in vivo-hydrolysable precursors thereof wherein constituent members are provided herein, and a choline esterase inhibitor or anti-inflammatory agent.

In some embodiments, the present invention provides a method of treating or preventing Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration, or any other disease, disorder, or condition described herein, by administering to a mammal (including human) a compound of the present invention, and an atypical antipsychotic agent. Atypical antipsychotic agents includes, but not limited to, Olanzapine (marketed as Zyprexa), Aripiprazole (marketed as Abilify), Risperidone (marketed as Risperdal), Quetiapine (marketed as Seroquel), Clozapine (marketed as Clozaril), Ziprasidone (marketed as Geodon) and Olanzapine/Fluoxetine (marketed as Symbyax).

In some embodiments, the mammal or human being treated with a compound of the present invention, has been diagnosed with a particular disease or disorder, such as those described herein. In these cases, the mammal or human being treated is in need of such treatment. Diagnosis, however, need not be previously performed.

The anti-dementia treatment defined herein may be applied as a sole therapy or may involve, in addition to the compound of the invention, conventional chemotherapy. Such chemotherapy may include one or more of the following categories of agents: acetyl cholinesterase inhibitors, anti-inflammatory agents, cognitive and/or memory enhancing agents or atypical antipsychotic agents.

Such conjoint treatment may be achieved by way of the simultaneous, sequential or separate dosing of the individual components of the treatment. Such combination products employ the compounds of this invention.

Cognitive enhancing agents memory enhancing agents and choline esterase inhibitors includes, but not limited to, onepezil (Aricept), galantamine (Reminyl or Razadyne), rivastigmine (Exelon), tacrine (Cognex) and memantine (Namenda, Axura or Ebixa).

The present invention also includes pharmaceutical compositions which contain, as the active ingredient, one or more of the compounds of the invention herein together with at least one pharmaceutically acceptable carrier, diluent or excipient.

When used for pharmaceutical compositions, medicaments, manufacture of a medicament, inhibiting activity of BACE, or treating or preventing Aβ-related pathologies, compounds of the present invention include the compounds of any of the formulas described herein, and pharmaceutically acceptable salts, tautomers and in vivo-hydrolysable precursors thereof. Compounds of the present invention further include hydrates and solvates.

The definitions set forth in this application are intended to clarify terms used throughout this application. The term “herein” means the entire application.

As used in this application, the term “optionally substituted,” as used herein, means that substitution is optional and therefore it is possible for the designated atom or moiety to be unsubstituted. In the event a substitution is desired then such substitution means that any number of hydrogens on the designated atom or moiety is replaced with a selection from the indicated group, provided that the normal valency of the designated atom or moiety is not exceeded, and that the substitution results in a stable compound. For example, if a methyl group (i.e., CH₃) is optionally substituted, then 3 hydrogens on the carbon atom can be replaced. Examples of suitable substituents include, but are not limited to: halogen, CN, NH₂, OH, SO, SO₂, COOH, OC₁₋₆alkyl, CH₂OH, SO₂H, C₁₋₆alkyl, OC₁₋₆alkyl, C(═O)C₁₋₆alkyl, C(═O)OC₁₋₆alkyl, C(═O)NH₂, C(═O)NHC₁₋₆alkyl, C(═O)N(C₁₋₆alkyl)2, SO₂C₁₋₆alkyl, SO₂NHC₁₋₆alkyl, SO₂N(C₁₋₆alkyl)2, NH(C₁₋₆alkyl), N(C₁₋₆alkyl)2, NHC(═O)C₁₋₆alkyl, NC(═O)(C₁₋₆alkyl)₂, C₅₋₆aryl, OC₅₋₆aryl, C(═O)C₅₋₆aryl, C(═O)OC₅₋₆aryl, C(═O)NHC₅₋₆aryl, C(═O)N(C₅₋₆aryl)2, SO₂C₅₋₆aryl, SO₂NHC₅₋₆aryl, SO₂N(C₅₋₆aryl)₂, NH(C₅₋₆aryl), N(C₅₋₆aryl)2, NC(═O)C₅₋₆aryl, NC(═O)(C₅₋₆aryl)₂, C₅₋₆heterocyclyl, OC₅₋₆heterocyclyl, C(═O)C₅₋₆heterocyclyl, C(═O)OC₅₋₆heterocyclyl, C(═O)NHC₅₋₆heterocyclyl, C(═O)N(C₅₋₆heterocyclyl)2, SO₂C₅₋₆heterocyclyl, SO₂NHC₅₋₆heterocyclyl, SO₂N(C₅₋₆heterocyclyl)₂, NH(C₅₋₆heterocyclyl), N(C₅₋₆heterocycyl)₂, NC(═O)C₅₋₆heterocyclyl, NC(═O)(C₅₋₆heterocyclyl)₂.

A variety of compounds in the present invention may exist in particular geometric or stereoisomeric forms. The present invention takes into account all such compounds, including cis- and trans isomers, R- and S-enantiomers, diastereomers, (D)-isomers, (L)-isomers, the racemic mixtures thereof, and other mixtures thereof, as being covered within the scope of this invention. Additional asymmetric carbon atoms may be present in a substituent such as an alkyl group. All such isomers, as well as mixtures thereof, are intended to be included in this invention. The compounds herein described may have asymmetric centers. Compounds of the present invention containing an asymmetrically substituted atom may be isolated in optically active or racemic forms. It is well known in the art how to prepare optically active forms, such as by resolution of racemic forms or by synthesis from optically active starting materials. When required, separation of the racemic material can be achieved by methods known in the art. Many geometric isomers of olefins, C═N double bonds, and the like can also be present in the compounds described herein, and all such stable isomers are contemplated in the present invention. Cis and trans geometric isomers of the compounds of the present invention are described and may be isolated as a mixture of isomers or as separated isomeric forms. All chiral, diastereomeric, racemic forms and all geometric isomeric forms of a structure are intended, unless the specific stereochemistry or isomeric form is specifically indicated.

When a bond to a substituent is shown to cross a bond connecting two atoms in a ring, then such substituent may be bonded to any atom on the ring. When a substituent is listed without indicating the atom via which such substituent is bonded to the rest of the compound of a given formula, then such substituent may be bonded via any atom in such substituent. Combinations of substituents and/or variables are permissible only if such combinations result in stable compounds.

As used herein, “alkyl”, “alkylenyl” or “alkylene” used alone or as a suffix or prefix, is intended to include both branched and straight-chain saturated aliphatic hydrocarbon groups having from 1 to 12 carbon atoms or if a specified number of carbon atoms is provided then that specific number would be intended. For example “C₁₋₆alkyl” denotes alkyl having 1, 2, 3, 4, 5 or 6 carbon atoms. Examples of alkyl include, but are not limited to, methyl, ethyl, n-propyl, i-propyl, n-butyl, i-butyl, sec-butyl, t-butyl, pentyl, and hexyl. As used herein, “C₁₋₃alkyl”, whether a terminal substituent or an alkylene (or alkylenyl) group linking two substituents, is understood to specifically include both branched and straight-chain methyl, ethyl, and propyl.

As used herein, “alkenyl” refers to an alkyl group having one or more double carbon-carbon bonds. Example alkenyl groups include ethenyl, propenyl, cyclohexenyl, and the like. The term “alkenylenyl” refers to a divalent linking alkenyl group.

As used herein, “alkynyl” refers to an alkyl group having one or more triple carbon-carbon bonds. Example alkynyl groups include ethynyl, propynyl, and the like. The term “alkynylenyl” refers to a divalent linking alkynyl group.

As used herein, “aromatic” refers to hydrocarbyl groups having one or more polyunsaturated carbon rings having aromatic characters, (e.g., 4n+2 delocalized electrons) and comprising up to about 14 carbon atoms.

As used herein, the term “aryl” refers to an aromatic ring structure made up of from 5 to 14 carbon atoms. Ring structures containing 5, 6, 7 and 8 carbon atoms would be single-ring aromatic groups, for example, phenyl. Ring structures containing 8, 9, 10, 11, 12, 13, or 14 would be a polycyclic moiety in which at least one carbon is common to any two adjoining rings therein (for example, the rings are “fused rings”), for example naphthyl. The aromatic ring can be substituted at one or more ring positions with such substituents as described above. The term “aryl” also includes polycyclic ring systems having two or more cyclic rings in which two or more carbons are common to two adjoining rings (the rings are “fused rings”) wherein at least one of the rings is aromatic, for example, the other cyclic rings can be cycloalkyls, cycloalkenyls or cycloalkynyls. The terms ortho, meta and para apply to 1,2-, 1,3- and 1,4-disubstituted benzenes, respectively. For example, the names 1,2-dimethylbenzene and ortho-dimethylbenzene are synonymous.

As used herein, “cycloalkyl” refers to non-aromatic cyclic hydrocarbons including cyclized alkyl, alkenyl, and alkynyl groups, having the specified number of carbon atoms. Cycloalkyl groups can include mono- or polycyclic (e.g., having 2, 3 or 4 fused or bridged rings) groups. Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, adamantyl, and the like. Also included in the definition of cycloalkyl are moieties that have one or more aromatic rings fused (i.e., having a bond in common with) to the cycloalkyl ring, for example, benzo derivatives of cyclopentane (i.e., indanyl), cyclopentene, cyclohexane, and the like. The term “cycloalkyl” further includes saturated ring groups, having the specified number of carbon atoms. These may include fused or bridged polycyclic systems. Preferred cycloalkyls have from 3 to 10 carbon atoms in their ring structure, and more preferably have 3, 4, 5, and 6 carbons in the ring structure. For example, “C₃₋₆ cycloalkyl” denotes such groups as cyclopropyl, cyclobutyl, cyclopentyl, or cyclohexyl.

As used herein, “cycloalkenyl” refers to ring-containing hydrocarbyl groups having at least one carbon-carbon double bond in the ring, and having from 3 to 12 carbons atoms.

As used herein, “halo” or “halogen” refers to fluoro, chloro, bromo, and iodo. “Counterion” is used to represent a small, negatively or positively charged species such as chloride (Cl⁻), bromide (Br⁻), hydroxide (OH⁻), acetate (CH₃COO⁻), sulfate (SO₄²⁻), tosylate (CH₃-phenyl-SO₃⁻), benezensulfonate (phenyl-SO₃⁻), sodium ion (Na⁺), potassium (K⁺), ammonium (NH₄⁺), and the like.

As used herein, the term “heterocyclyl” or “heterocyclic” or “heterocycle” refers to a ring-containing monovalent and divalent structures having one or more heteroatoms, independently selected from N, O and S, as part of the ring structure and comprising from 3 to 20 atoms in the rings, more preferably 3- to 7-membered rings. The number of ring-forming atoms in heterocyclyl are given in ranges herein. For example, C₅₋₁₀ heterocyclyl refers to a ring structure comprising from 5 to 10 ring-forming atoms wherein at least one of the ring-forming atoms is N, O or S. Heterocyclic groups may be saturated or partially saturated or unsaturated, containing one or more double bonds, and heterocyclic groups may contain more than one ring as in the case of polycyclic systems. The heterocyclic rings described herein may be substituted on carbon or on a heteroatom atom if the resulting compound is stable. If specifically noted, nitrogen in the heterocyclyl may optionally be quaternized. It is understood that when the total number of S and O atoms in the heterocyclyl exceeds 1, then these heteroatoms are not adjacent to one another.

Examples of heterocyclyls include, but are not limited to, 1H-indazole, 2-pyrrolidonyl, 2H, 6H-1,5,2-dithiazinyl, 2H-pyrrolyl, 3H-indolyl, 4-piperidonyl, 4aH-carbazole, 4H-quinolizinyl, 6H-1,2,5-thiadiazinyl, acridinyl, azabicyclo, azetidine, azepane, aziridine, azocinyl, benzimidazolyl, benzodioxol, benzofuranyl, benzothiofuranyl, benzothiophenyl, benzoxazolyl, benzthiazolyl, benzotriazolyl, benzotetrazolyl, benzisoxazolyl, benzisothiazolyl, benzimidazalonyl, carbazolyl, 4aH-carbazolyl, b-carbolinyl, chromanyl, chromenyl, cinnolinyl, diazepane, decahydroquinolinyl, 2H,6H-1,5,2-dithiazinyl, dioxolane, furyl, 2,3-dihydrofuran, 2,5-dihydrofuran, dihydrofuro[2,3-b]tetrahydrofuran, furanyl, furazanyl, homopiperidinyl, imidazolidine, imidazolidinyl, imidazolinyl, imidazolyl, 1H-indazolyl, indolenyl, indolinyl, indolizinyl, indolyl, isobenzofuranyl, isochromanyl, isoindazolyl, isoindolinyl, isoindolyl, isoquinolinyl, isothiazolyl, isoxazolyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxirane, oxazolidinylperimidinyl, phenanthridinyl, phenanthrolinyl, phenarsazinyl, phenazinyl, phenothiazinyl, phenoxathiinyl, phenoxazinyl, phthalazinyl, piperazinyl, piperidinyl, pteridinyl, piperidonyl, 4-piperidonyl, purinyl, pyranyl, pyrrolidinyl, pyrroline, pyrrolidine, pyrazinyl, pyrazolidinyl, pyrazolinyl, pyrazolyl, pyridazinyl, pyridooxazole, pyridoimidazole, pyridothiazole, pyridinyl, N-oxide-pyridinyl, pyridyl, pyrimidinyl, pyrrolidinyl, pyrrolidinyl dione, pyrrolinyl, pyrrolyl, pyridine, quinazolinyl, quinolinyl, 4H-quinolizinyl, quinoxalinyl, quinuclidinyl, carbolinyl, tetrahydrofuranyl, tetramethylpiperidinyl, tetrahydroquinoline, tetrahydroisoquinolinyl, thiophane, thiotetrahydroquinolinyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, thiirane, triazinyl, 1,2,3-triazolyl, 1,2,4-triazolyl, 1,2,5-triazolyl, 1,3,4-triazolyl, xanthenyl.

As used herein, “heteroaryl” refers to an aromatic heterocycle having at least one heteroatom ring member such as sulfur, oxygen, or nitrogen. Heteroaryl groups include monocyclic and polycyclic (e.g., having 2, 3 or 4 fused rings) systems. Examples of heteroaryl groups include without limitation, pyridyl (i.e., pyridinyl), pyrimidinyl, pyrazinyl, pyridazinyl, triazinyl, furyl (i.e. furanyl), quinolyl, isoquinolyl, thienyl, imidazolyl, thiazolyl, indolyl, pyrryl, oxazolyl, benzofuryl, benzothienyl, benzthiazolyl, isoxazolyl, pyrazolyl, triazolyl, tetrazolyl, indazolyl, 1,2,4-thiadiazolyl, isothiazolyl, benzothienyl, purinyl, carbazolyl, benzimidazolyl, indolinyl, and the like. In some embodiments, the heteroaryl group has from 1 to about 20 carbon atoms, and in further embodiments from about 3 to about 20 carbon atoms. In some embodiments, the heteroaryl group contains 3 to about 14, 4 to about 14, 3 to about 7, or 5 to 6 ring-forming atoms. In some embodiments, the heteroaryl group has 1 to about 4, 1 to about 3, or 1 to 2 heteroatoms. In some embodiments, the heteroaryl group has 1 heteroatom.

As used herein, “alkoxy” or “alkyloxy” represents an alkyl group as defined above with the indicated number of carbon atoms attached through an oxygen bridge. Examples of alkoxy include, but are not limited to, methoxy, ethoxy, n-propoxy, isopropoxy, n-butoxy, isobutoxy, t-butoxy, n-pentoxy, isopentoxy, cyclopropylmethoxy, allyloxy and propargyloxy. Similarly, “alkylthio” or “thioalkoxy” represent an alkyl group as defined above with the indicated number of carbon atoms attached through a sulphur bridge.

As used herein, the term “carbonyl” is art recognized and includes such moieties as can be represented by the general formula:

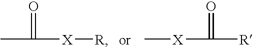

wherein X is a bond or represents an oxygen or sulfur, and R represents a hydrogen, an alkyl, an alkenyl, —(CH₂)ₘ—R″ or a pharmaceutically acceptable salt, R′ represents a hydrogen, an alkyl, an alkenyl or —(CH₂)ₘ—R″, where m is an integer less than or equal to ten, and R″ is alkyl, cycloalkyl, alkenyl, aryl, or heteroaryl. Where X is an oxygen and R and R′ is not hydrogen, the formula represents an “ester”. Where X is an oxygen, and R is as defined above, the moiety is referred to herein as a carboxyl group, and particularly when R′ is a hydrogen, the formula represents a “carboxylic acid.” Where X is oxygen, and R′ is a hydrogen, the formula represents a “formate.” In general, where the oxygen atom of the above formula is replaced by sulfur, the formula represents a “thiolcarbonyl” group. Where X is a sulfur and R and R′ is not hydrogen, the formula represents a “thiolester.” Where X is sulfur and R is hydrogen, the formula represents a “thiolcarboxylic acid.” Where X is sulfur and R′ is hydrogen, the formula represents a “thiolformate.” On the other hand, where X is a bond, and R is not a hydrogen, the above formula represents a “ketone” group. Where X is a bond, and R is hydrogen, the above formula is represents an “aldehyde” group.

As used herein, the term “sulfonyl” refers to a moiety that can be represented by the general formula:

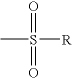

wherein R is represented by but not limited to hydrogen, alkyl, cycloalkyl, alkenyl, aryl, heteroaryl, aralkyl, or heteroaralkyl.

As used herein, some substituents are described in a combination of two or more groups. For example, the expression of “C(═O)C₃₋₉cycloalkylR^{d}” is meant to refer to a structure:

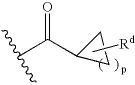

wherein p is 1, 2, 3, 4, 5, 6 or 7 (i.e., C₃₋₉cycloalkyl); the C₃₋₉cycloalkyl is substituted by R^{d}; and the point of attachment of the “C(═O)C₃₋₉cycloalkylR^{d}” is through the carbon atom of the carbonyl group, which is on the left of the expression.

As used herein, the phrase “protecting group” means temporary substituents which protect a potentially reactive functional group from undesired chemical transformations. Examples of such protecting groups include esters of carboxylic acids, silyl ethers of alcohols, and acetals and ketals of aldehydes and ketones respectively. The field of protecting group chemistry has been reviewed (Greene, T. W.; Wuts, P. G. M. Protective Groups in Organic Synthesis, 3^{rd} ed.; Wiley: New York, 1999).

As used herein, “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, “pharmaceutically acceptable salts” refer to derivatives of the disclosed compounds wherein the parent compound is modified by making acid or base salts thereof (i.e., also include counterions). Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, phosphoric, and the like; and the salts prepared from organic acids such as lactic, maleic, citric, benzoic, methanesulfonic, trifluoroacetic, and the like.

The pharmaceutically acceptable salts of the present invention can be synthesized from the parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; nonaqueous media like ether, ethyl acetate, ethanol, isopropanol, or acetonitrile can be used.

As used herein, “in vivo hydrolysable precursors” means an in vivo hydroysable (or cleavable) ester of a compound of any of the formulas described herein that contains a carboxy or a hydroxy group. For example amino acid esters, C₁₋₆ alkoxymethyl esters like methoxymethyl; C₁₋₆alkanoyloxymethyl esters like pivaloyloxymethyl; C₃₋₈cycloalkoxycarbonyloxy C₁₋₆alkyl esters like 1-cyclohexylcarbonyloxyethyl, acetoxymethoxy, or phosphoramidic cyclic esters.

As used herein, “tautomer” means other structural isomers that exist in equilibrium resulting from the migration of a hydrogen atom. For example, keto-enol tautomerism where the resulting compound has the properties of both a ketone and an unsaturated alcohol.

As used herein “stable compound” and “stable structure” are meant to indicate a compound that is sufficiently robust to survive isolation to a useful degree of purity from a reaction mixture, and formulation into an efficacious therapeutic agent.

The present invention further includes isotopically-labeled compounds of the invention. An “isotopically” or “radio-labeled” compound is a compound of the invention where one or more atoms are replaced or substituted by an atom having an atomic mass or mass number different from the atomic mass or mass number typically found in nature (i.e., naturally occurring). Suitable radionuclides that may be incorporated in compounds of the present invention include but are not limited to ²H (also written as D for deuterium), ³H (also written as T for tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I and ¹³¹I. The radionuclide that is incorporated in the instant radio-labeled compounds will depend on the specific application of that radio-labeled compound. For example, for in vitro receptor labeling and competition assays, compounds that incorporate ³H, ¹⁴C, ⁸²Br, ¹²⁵I, ¹³¹I, ³⁵S or will generally be most useful. For radio-imaging applications ¹¹C, ¹⁸F, ¹²⁵I, ¹²³I, ¹²⁴I, ¹³¹I, ⁷⁵Br, ⁷⁶Br or ⁷⁷Br will generally be most useful.

It is understood that a “radio-labeled compound” is a compound that has incorporated at least one radionuclide. In some embodiments the radionuclide is selected from the group consisting of ³H, ¹⁴C, ¹²⁵I, ³⁵S and ⁸²Br.

The antidementia treatment defined herein may be applied as a sole therapy or may involve, in addition to the compound of the invention, conventional chemotherapy.
Such conjoint treatment may be achieved by way of the simultaneous, sequential or separate dosing of the individual components of the treatment. Such combination products employ the compounds of this invention.

Compounds of the present invention may be administered orally, parenteral, buccal, vaginal, rectal, inhalation, insufflation, sublingually, intramuscularly, subcutaneously, topically, intranasally, intraperitoneally, intrathoracially, intravenously, epidurally, intrathecally, intracerebroventricularly and by injection into the joints.

The dosage will depend on the route of administration, the severity of the disease, age and weight of the patient and other factors normally considered by the attending physician, when determining the individual regimen and dosage level as the most appropriate for a particular patient.

An effective amount of a compound of the present invention for use in therapy of dementia is an amount sufficient to symptomatically relieve in a warm-blooded animal, particularly a human the symptoms of dementia, to slow the progression of dementia, or to reduce in patients with symptoms of dementia the risk of getting worse.

For preparing pharmaceutical compositions from the compounds of this invention, inert, pharmaceutically acceptable carriers can be either solid or liquid. Solid form preparations include powders, tablets, dispersible granules, capsules, cachets, and suppositories.

A solid carrier can be one or more substances, which may also act as diluents, flavoring agents, solubilizers, lubricants, suspending agents, binders, or tablet disintegrating agents; it can also be an encapsulating material.

In powders, the carrier is a finely divided solid, which is in a mixture with the finely divided active component. In tablets, the active component is mixed with the carrier having the necessary binding properties in suitable proportions and compacted in the shape and size desired.

For preparing suppository compositions, a low-melting wax such as a mixture of fatty acid glycerides and cocoa butter is first melted and the active ingredient is dispersed therein by, for example, stirring. The molten homogeneous mixture is then poured into convenient sized molds and allowed to cool and solidify.

Suitable carriers include magnesium carbonate, magnesium stearate, talc, lactose, sugar, pectin, dextrin, starch, tragacanth, methyl cellulose, sodium carboxymethyl cellulose, a low-melting wax, cocoa butter, and the like.

Some of the compounds of the present invention are capable of forming salts with various inorganic and organic acids and bases and such salts are also within the scope of this invention. For example, such conventional non-toxic salts include those derived from inorganic acids such as hydrochloric, phosphoric, and the like; and the salts prepared from organic acids such as lactic, maleic, citric, benzoic, methanesulfonic, trifluoroacetate and the like.

In some embodiments, the present invention provides a compound of any of the formulas described herein or a pharmaceutically acceptable salt thereof for the therapeutic treatment (including prophylactic treatment) of mammals including humans, it is normally formulated in accordance with standard pharmaceutical practice as a pharmaceutical composition.

In addition to the compounds of the present invention, the pharmaceutical composition of this invention may also contain, or be co-administered (simultaneously or sequentially) with, one or more pharmacological agents of value in treating one or more disease conditions referred to herein.

The term composition is intended to include the formulation of the active component or a pharmaceutically acceptable salt with a pharmaceutically acceptable carrier. For example this invention may be formulated by means known in the art into the form of, for example, tablets, capsules, aqueous or oily solutions, suspensions, emulsions, creams, ointments, gels, nasal sprays, suppositories, finely divided powders or aerosols or nebulisers for inhalation, and for parenteral use (including intravenous, intramuscular or infusion) sterile aqueous or oily solutions or suspensions or sterile emulsions.

Liquid form compositions include solutions, suspensions, and emulsions. Sterile water or water-propylene glycol solutions of the active compounds may be mentioned as an example of liquid preparations suitable for parenteral administration. Liquid compositions can also be formulated in solution in aqueous polyethylene glycol solution. Aqueous solutions for oral administration can be prepared by dissolving the active component in water and adding suitable colorants, flavoring agents, stabilizers, and thickening agents as desired. Aqueous suspensions for oral use can be made by dispersing the finely divided active component in water together with a viscous material such as natural synthetic gums, resins, methyl cellulose, sodium carboxymethyl cellulose, and other suspending agents known to the pharmaceutical formulation art.

The pharmaceutical compositions can be in unit dosage form. In such form, the composition is divided into unit doses containing appropriate quantities of the active component. The unit dosage form can be a packaged preparation, the package containing discrete quantities of the preparations, for example, packeted tablets, capsules, and powders in vials or ampoules. The unit dosage form can also be a capsule, cachet, or tablet itself, or it can be the appropriate number of any of these packaged forms. Compositions may be formulated for any suitable route and means of administration. Pharmaceutically acceptable carriers or diluents include those used in formulations suitable for oral, rectal, nasal, topical (including buccal and sublingual), vaginal or parenteral (including subcutaneous, intramuscular, intravenous, intradermal, intrathecal and epidural) administration. The formulations may conveniently be presented in unit dosage form and may be prepared by any of the methods well known in the art of pharmacy.

For solid compositions, conventional non-toxic solid carriers include, for example, pharmaceutical grades of mannitol, lactose, cellulose, cellulose derivatives, starch, magnesium stearate, sodium saccharin, talcum, glucose, sucrose, magnesium carbonate, and the like may be used. Liquid pharmaceutically administrable compositions can, for example, be prepared by dissolving, dispersing, etc, an active compound as defined above and optional pharmaceutical adjuvants in a carrier, such as, for example, water, saline aqueous dextrose, glycerol, ethanol, and the like, to thereby form a solution or suspension. If desired, the pharmaceutical composition to be administered may also contain minor amounts of non-toxic auxiliary substances such as wetting or emulsifying agents, pH buffering agents and the like, for example, sodium acetate, sorbitan monolaurate, triethanolamine sodium acetate, sorbitan monolaurate, triethanolamine oleate, etc. Actual methods of preparing such dosage forms are known, or will be apparent, to those skilled in this art; for example, see Remington's Pharmaceutical Sciences, Mack Publishing Company, Easton, Pa., 15th Edition, 1975.

The compounds of the invention may be derivatised in various ways. As used herein “derivatives” of the compounds includes salts (e.g. pharmaceutically acceptable salts), any complexes (e.g. inclusion complexes or clathrates with compounds such as cyclodextrins, or coordination complexes with metal ions such as Mn²⁺ and Zn²⁺), esters such as in vivo hydrolysable esters, free acids or bases, polymorphic forms of the compounds, solvates (e.g. hydrates), prodrugs or lipids, coupling partners and protecting groups. By “prodrugs” is meant for example any compound that is converted in vivo into a biologically active compound.

Salts of the compounds of the invention are preferably physiologically well tolerated and non toxic. Many examples of salts are known to those skilled in the art. All such salts are within the scope of this invention, and references to compounds include the salt forms of the compounds.

Compounds having acidic groups, such as carboxylate, phosphates or sulfates, can form salts with alkaline or alkaline earth metals such as Na, K, Mg and Ca, and with organic amines such as triethylamine and Tris (2-hydroxyethyl)amine. Salts can be formed between compounds with basic groups, e.g. amines, with inorganic acids such as hydrochloric acid, phosphoric acid or sulfuric acid, or organic acids such as acetic acid, citric acid, benzoic acid, fumaric acid, or tartaric acid. Compounds having both acidic and basic groups can form internal salts.

Acid addition salts may be formed with a wide variety of acids, both inorganic and organic. Examples of acid addition salts include salts formed with hydrochloric, hydriodic, phosphoric, nitric, sulphuric, citric, lactic, succinic, maleic, malic, isethionic, fumaric, benzenesulphonic, toluenesulphonic, methanesulphonic, ethanesulphonic, naphthalenesulphonic, valeric, acetic, propanoic, butanoic, malonic, glucuronic and lactobionic acids.

If the compound is anionic, or has a functional group which may be anionic (e.g., COOH may be COO), then a salt may be formed with a suitable cation. Examples of suitable inorganic cations include, but are not limited to, alkali metal ions such as Na⁺ and K⁺, alkaline earth cations such as Ca²⁺ and Mg²⁺, and other cations such as Al³⁺. Examples of suitable organic cations include, but are not limited to, ammonium ion (i.e., NH₄⁺) and substituted ammonium ions (e.g., NH₃R⁺, NH₂R₂⁺, NHR₃⁺, NR₄⁺). Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

Where the compounds contain an amine function, these may form quaternary ammonium salts, for example by reaction with an alkylating agent according to methods well known to the skilled person. Such quaternary ammonium compounds are within the scope of the invention.

Compounds containing an amine function may also form N-oxides. A reference herein to a compound that contains an amine function also includes the N-oxide.

Where a compound contains several amine functions, one or more than one nitrogen atom may be oxidised to form an N-oxide. Particular examples of N-oxides are the N-oxides of a tertiary amine or a nitrogen atom of a nitrogen-containing heterocycle.

N-Oxides can be formed by treatment of the corresponding amine with an oxidizing agent such as hydrogen peroxide or a per-acid (e.g. a peroxycarboxylic acid), see for example Advanced Organic Chemistry, by Jerry March, 4^{th} Edition, Wiley Interscience, pages. More particularly, N-oxides can be made by the procedure of L. W. Deady (Syn. Comm. 1977, 7, 509-514) in which the amine compound is reacted with m-chloroperoxybenzoic acid (MCPBA), for example, in an inert solvent such as dichloromethane.

Esters can be formed between hydroxyl or carboxylic acid groups present in the compound and an appropriate carboxylic acid or alcohol reaction partner, using techniques well known in the art. Examples of esters are compounds containing the group C(═O)OR, wherein R is an ester substituent, for example, a C₁₋₇ alkyl group, a C₃₋₂₀ heterocyclyl group, or a C₅₋₂₀ aryl group, preferably a C₁₋₇ alkyl group. Particular examples of ester groups include, but are not limited to, C(═O)OCH₃, C(═O)OCH₂CH₃, C(═O)OC(CH₃)₃, and —C(═O)OPh. Examples of acyloxy (reverse ester) groups are represented by OC(═O)R, wherein R is an acyloxy substituent, for example, a C₁₋₇ alkyl group, a C₃₋₂₀ heterocyclyl group, or a C₅₋₂₀ aryl group, preferably a C₁₋₇ alkyl group. Particular examples of acyloxy groups include, but are not limited to, OC(═O)CH₃ (acetoxy), OC(═O)CH₂CH₃, OC(═O)C(CH₃)₃, OC(═O)Ph, and OC(═O)CH₂Ph.

Derivatives which are prodrugs of the compounds are convertible in vivo or in vitro into one of the parent compounds. Typically, at least one of the biological activities of compound will be reduced in the prodrug form of the compound, and can be activated by conversion of the prodrug to release the compound or a metabolite of it. Some prodrugs are esters of the active compound (e.g., a physiologically acceptable metabolically labile ester). During metabolism, the ester group (—C(═O)OR) is cleaved to yield the active drug. Such esters may be formed by esterification, for example, of any of the carboxylic acid groups (—C(═O)OH) in the parent compound, with, where appropriate, prior protection of any other reactive groups present in the parent compound, followed by deprotection if required.

Examples of such metabolically labile esters include those of the formula —C(═O)O wherein R is: C₁₋₇alkyl (e.g., Me, Et, -nPr, -iPr, -nBu, -sBu, -iBu, tBu); C₁₇aminoalkyl (e.g., aminoethyl; 2-(N,N-diethylamino)ethyl; 2(4morpholino)ethyl); and acyloxy-C₁₇alkyl (e.g., acyloxymethyl; acyloxyethyl; pivaloyloxymethyl; acetoxymethyl; 1 acetoxyethyl; 1-(1-methoxy-1-methyl)ethyl-carbonyloxyethyl; 1-(benzoyloxy)ethyl; isopropoxy-carbonyloxymethyl; 1isopropoxy-carbonyloxyethyl; cyclohexyl-carbonyloxymethyl; 1cyclohexyl-carbonyloxyethyl; cyclohexyloxy-carbonyloxymethyl; 1-cyclohexyloxy-carbonyloxyethyl; (4-tetrahydropyranyloxy) carbonyloxymethyl; 1-(4-tetrahydropyranyloxy)carbonyloxyethyl; (4-tetrahydropyranyl)carbonyloxymethyl; and 1(4tetrahydropyranyl)carbonyloxyethyl).

Also, some prodrugs are activated enzymatically to yield the active compound, or a compound which, upon further chemical reaction, yields the active compound (for example, as in ADEPT, GDEPT, LIDEPT, etc.). For example, the prodrug may be a sugar derivative or other glycoside conjugate, or may be an amino acid ester derivative.

Other derivatives include coupling partners of the compounds in which the compounds is linked to a coupling partner, e.g. by being chemically coupled to the compound or physically associated with it. Examples of coupling partners include a label or reporter molecule, a supporting substrate, a carrier or transport molecule, an effector, a drug, an antibody or an inhibitor. Coupling partners can be covalently linked to compounds of the invention via an appropriate functional group on the compound such as a hydroxyl group, a carboxyl group or an amino group. Other derivatives include formulating the compounds with liposomes.

Where the compounds contain chiral centres, all individual optical forms such as enantiomers, epimers and diastereoisomers, as well as racemic mixtures of the compounds are within the scope of the invention.

Compounds may exist in a number of different geometric isomeric, and tautomeric forms and references to compounds include all such forms. For the avoidance of doubt, where a compound can exist in one of several geometric isomeric or tautomeric forms and only one is specifically described or shown, all others are nevertheless embraced by the scope of this invention.

The quantity of the compound to be administered will vary for the patient being treated and will vary from about 100 ng/kg of body weight to 100 mg/kg of body weight per day and preferably will be from 10 pg/kg to 10 mg/kg per day. For instance, dosages can be readily ascertained by those skilled in the art from this disclosure and the knowledge in the art. Thus, the skilled artisan can readily determine the amount of compound and optional additives, vehicles, and/or carrier in compositions and to be administered in methods of the invention.

Compounds of the present invention have been shown to inhibit beta secretase (including BACE) activity in vitro. Inhibitors of beta secretase have been shown to be useful in blocking formation or aggregation of Aβ peptide and therefore have a beneficial effects in treatment of Alzheimer's Disease and other neurodegenerative diseases associated with elevated levels and/or deposition of Aβ peptide. Therefore it is believed that the compounds of the present invention may be used for the treatment of Alzheimer disease and disease associated with dementia. Hence compounds of the present invention and their salts are expected to be active against age-related diseases such as Alzheimer, as well as other Aβ related pathologies such as Down's syndrome and b-amyloid angiopathy. It is expected that the compounds of the present invention would most likely be used in combination with a broad range of cognition deficit enhancement agents but could also be used as a single agent.

Generally, the compounds of the present invention have been identified in one or both assays described below as having an IC₅₀ value of 100 micromolar or less.

### IGEN Assay

Enzyme is diluted 1:30 in 40 mM MES pH 5.0. Stock substrate is diluted to 12 μM in 40 mM MES pH 5.0. PALMEB solution is added to the substrate solution (1:100 dilution). DMSO stock solutions of compounds or DMSO alone are diluted to the desired concentration in 40 mM MES pH 5.0. The assay is done in a 96 well PCR plate from Nunc. Compound in DMSO (3 μL) is added to the plate then enzyme is added (27 μL) and pre-incubated with compound for 5 minutes. Then the reaction is started with substrate (30 μL). The final dilution of enzyme is 1:60; the final concentration of substrate is 6 μM (Km is 150 μM). After a 20 minute reaction at room temperature, the reaction is stopped by removing 10 μl of the reaction mix and diluting it 1:25 in 0.20M Tris pH 8.0. The compounds are added to the plate by hand then all the rest of the liquid handling is done on the CyBi-well instrument.

All antibodies and the streptavidin coated beads are diluted into PBS containing 0.5% BSA and 0.5% Tween20. The product is quantified by adding 50 μL of a 1:5000 dilution of the neoepitope antibody to 50 μL of the 1:25 dilution of the reaction mix. Then, 100 μL of PBS (0.5% BSA, 0.5% Tween20) containing 0.2 mg/ml IGEN beads and a 1:5000 dilution of ruthinylated goat anti-rabbit (Ru-Gar) antibody is added. The final dilution of neoepitope antibody is 1:20,000, the final dilution of Ru-GAR is 1:10,000 and the final concentration of beads is 0.1 mg/ml. The mixture is read on the IGEN instrument with the CindyAB40 program after a 2-hour incubation at room temperature. Addition of DMSO alone is used to define the 100% activity. 20 μM control inhibitor is used to define 0% of control activity and 100 nM inhibitor defines 50% control of control activity in single-poke assays. Control inhibitor is also used in dose response assays with an IC50 of 100 nM.

### Fluorescent Assay

Enzyme is diluted 1:30 in 40 mM MES pH 5.0. Stock substrate is diluted to 30 μM in 40 mM MES pH 5.0. PALMEB solution is added to the substrate solution (1:100 dilution). Enzyme and substrate stock solutions are kept on ice until the placed in the stock plates. The Platemate-plus instrument is used to do all liquid handling. Enzyme (9 μL) is added to the plate then 1 μL of compound in DMSO is added and pre-incubated for 5 minutes. When a dose response curve is being tested for a compound, the dilutions are done in neat DMSO and the DMSO stocks are added as described above. Substrate (10 μL) is added and the reaction proceeds in the dark for 1 hour at room temperature. The assay is done in a Corning 384 well round bottom, low volume, non-binding surface (Corning #3676). The final dilution of enzyme is 1:60; the final concentration of substrate is 15 μM (Km of 25 AI). The fluorescence of the product is measured on a Victor II plate reader with an excitation wavelength of 360 nm and an emission wavelength of 485 nm using the protocol labeled Edans peptide. The DMSO control defines the 100% activity level and 0% activity is defined by using 50 μM of the control inhibitor, which completely blocks enzyme function. The control inhibitor is also used in dose response assays and has an IC50 of 95 nM.

### Beta-Secretase Whole Cell Assay

### Generation of HEK-Fc33-1:

The cDNA encoding full length BACE was fused in frame with a three amino acid linker (Ala-Val-Thr) to the Fc portion of the human IgG1 starting at amino acid 104. The BACE-Fc construct was then cloned into a GFP/pGEN-IRES-neoK vector (a proprietary vector of AstraZeneca) for protein expression in mammalian cells. The expression vector was stably transfected into HEK-293 cells using a calcium phosphate method. Colonies were selected with 250 μg/mL of G-418. Limited dilution cloning was performed to generate homogeneous cell lines. Clones were characterized by levels of APP expression and Aβ secreted in the conditioned media using an ELISA assay developed in-house. Aβ secretion of BACE/Fc clone Fc33-1 was moderate.

### Cell Culture:

HEK293 cells stably expressing human BACE (HEK-Fc33) were grown at 37° C. in DMEM containing 10% heat-inhibited FBS, 0.5 mg/mL antibiotic-antimycotic solution, and 0.05 mg/mL of the selection antibiotic G-418.

### Aβ40 Release Assay:

Cells were harvested when between 80 to 90% confluent. 100 μL of cells at a cell density of 1.5 million/mL were added to a white 96-well cell culture plate with clear flat bottom (Costar 3610), or a clear, flat bottom 96-well cell culture plate (Costar 3595), containing 100 μL of inhibitor in cell culture medium with DMSO at a final concentration of 1%. After the plate was incubated at 37° C. for 24 h, 100 μL cell medium was transferred to a round bottom 96-well plate (Costar 3365) to quantify Aβ40 levels. The cell culture plates were saved for ATP assay as described in ATP assay below. To each well of the round bottom plate, 50 μL of detection solution containing 0.2 μg/mL of the RαAβ40 antibody and 0.25 μg/mL of a biotinylated 4G8 antibody (prepared in DPBS with 0.5% BSA and 0.5% Tween-20) was added and incubated at 4° C. for at least 7 h. Then a 50 μL solution (prepared in the same buffer as above) containing 0.062 μg/mL of a ruthenylated goat anti-rabbit antibody and 0.125 mg/mL of streptavidin coated Dynabeads was added per well. The plate was shaken at 22° C. on a plate shaker for 1 h, and then the plates were then measured for ECL counts in an IGEN M8 Analyzer. Aβ standard curves were obtained with 2-fold serial dilution of an Aβ stock solution of known concentration in the same cell culture medium used in cell-based assays.

### ATP Assay:

As indicated above, after transferring 100 μL medium from cell culture plates for Aβ40 detection, the plates, which still contained cells, were saved for cytotoxicity assays by using the assay kit (ViaLight™ Plus) from Cambrex BioScience that measures total cellular ATP. Briefly, to each well of the plates, 50 μL cell lysis reagent was added. The plates were incubated at room temperature for 10 min. Two min following addition of 100 μL reconstituted ViaLight™ Plus reagent for ATP measurement, the luminescence of each well was measured in an LJL plate reader or Wallac Topcount.

### BACE Biacore Protocol

### Sensor Chip Preparation:

BACE was assayed on a Biacore3000 instrument by attaching either a peptidic transition state isostere (TSI) or a scrambled version of the peptidic TSI to the surface of a Biacore CM5 sensor chip. The surface of a CM5 sensor chip has 4 distinct channels that can be used to couple the peptides. The scrambled peptide KFES-statine-ETIAEVENV was coupled to channel 1 and the TSI inhibitor KTEEISEVN-statine-VAEF was couple to channel 2 of the same chip. The two peptides were dissolved at 0.2 mg/ml in 20 mM Na Acetate pH 4.5, and then the solutions were centrifuged at 14K rpm to remove any particulates. Carboxyl groups on the dextran layer were activated by injecting a one to one mixture of 0.5M N-ethyl-N′ (3-dimethylaminopropyl)-carbodiimide (EDC) and 0.5M N-hydroxysuccinimide (NHS) at 5 μL/minute for 7 minutes. Then the stock solution of the control peptide was injected in channel 1 for 7 minutes at 5 μL/min., and then the remaining activated carboxyl groups were blocked by injecting 1M ethanolamine for 7 minutes at 5 μL/minute.

### Assay Protocol:

The BACE Biacore assay was done by diluting BACE to 0.5 μM in Na Acetate buffer at pH 4.5 (running buffer minus DMSO). The diluted BACE was mixed with DMSO or compound diluted in DMSO at a final concentration of 5% DMSO. The BACE/inhibitor mixture was incubated for 1 hour at 4° C. then injected over channel 1 and 2 of the CM5 Biacore chip at a rate of 20 μL/minute. As BACE bound to the chip the signal was measured in response units (RU). BACE binding to the TSI inhibitor on channel 2 gave a certain signal. The presence of a BACE inhibitor reduced the signal by binding to BACE and inhibiting the interaction with the peptidic TSI on the chip. Any binding to channel 1 was non-specific and was subtracted from the channel 2 responses. The DMSO control was defined as 100% and the effect of the compound was reported as percent inhibition of the DMSO control.

hERG Assay

### Cell Culture

The hERG-expressing Chinese hamster ovary K1 (CHO) cells described by (Persson, Carlsson, Duker, & Jacobson, 2005) were grown to semi-confluence at 37° C. in a humidified environment (5% CO₂) in F-12 Ham medium containing L-glutamine, 10% foetal calf serum (FCS) and 0.6 mg/ml hygromycin (all Sigma-Aldrich). Prior to use, the monolayer was washed using a pre-warmed (37° C.) 3 ml aliquot of Versene 1:5,000 (Invitrogen). After aspiration of this solution the flask was incubated at 37° C. in an incubator with a further 2 ml of Versene 1:5,000 for a period of 6 minutes. Cells were then detached from the bottom of the flask by gentle tapping and 10 ml of Dulbecco's Phosphate-Buffered Saline containing calcium (0.9 mM) and magnesium (0.5 mM) (PBS; Invitrogen) was then added to the flask and aspirated into a 15 ml centrifuge tube prior to centrifugation (50 g, for 4 mins). The resulting supernatant was discarded and the pellet gently re-suspended in 3 ml of PBS. A 0.5 ml aliquot of cell suspension was removed and the number of viable cells (based on trypan blue exclusion) was determined in an automated reader (Cedex; Inovatis) so that the cell re-suspension volume could be adjusted with PBS to give the desired final cell concentration. It is the cell concentration at this point in the assay that is quoted when referring to this parameter. CHO-Kv1.5 cells, which were used to adjust the voltage offset on IonWorks™ HT, were maintained and prepared for use in the same way.

### Electrophysiology

The principles and operation of this device have been described by (Schroeder, Neagle, Trezise, & Worley, 2003). Briefly, the technology is based on a 384-well plate (PatchPlate™) in which a recording is attempted in each well by using suction to position and hold a cell on a small hole separating two isolated fluid chambers. Once sealing has taken place, the solution on the underside of the PatchPlate™ is changed to one containing amphotericin B. This permeablises the patch of cell membrane covering the hole in each well and, in effect, allows a perforated, whole-cell patch clamp recording to be made.

A β-test IonWorks™ HT from Essen Instrument was used. There is no capability to warm solutions in this device hence it was operated at room temperature (˜21° C.), as follows. The reservoir in the “Buffer” position was loaded with 4 ml of PBS and that in the “Cells” position with the CHO-hERG cell suspension described above. A 96-well plate (V-bottom, Greiner Bio-one) containing the compounds to be tested (at 3-fold above their final test concentration) was placed in the “Plate 1” position and a PatchPlate™ was clamped into the PatchPlate™ station. Each compound plate was laid-out in 12 columns to enable ten, 8-point concentration-effect curves to be constructed; the remaining two columns on the plate were taken up with vehicle (final concentration 0.33% DMSO), to define the assay baseline, and a supra-maximal blocking concentration of cisapride (final concentration 10 μM) to define the 100% inhibition level. The fluidics-head (F-Head) of IonWorks™ HT then added 3.5 μl of PBS to each well of the PatchPlate™ and its underside was perfused with “internal” solution that had the following composition (in mM): K-Gluconate 100, KCl 40, MgCl₂ 3.2, EGTA 3 and HEPES 5 (all Sigma-Aldrich; pH 7.25-7.30 using 10 M KOH). After priming and de-bubbling, the electronics-head (E-head) then moved round the PatchPlate™ performing a hole test (i.e. applying a voltage pulse to determine whether the hole in each well was open). The F-head then dispensed 3.5 μl of the cell suspension described above into each well of the PatchPlate™ and the cells were given 200 seconds to reach and seal to the hole in each well. Following this, the E-head moved round the PatchPlate™ to determine the seal resistance obtained in each well. Next, the solution on the underside of the PatchPlate™ was changed to “access” solution that had the following composition (in mM): KCl 140, EGTA 1, MgCl₂ 1 and HEPES 20 (pH 7.25-7.30 using 10 M KOH) plus 100 μg/ml of amphotericin B (Sigma-Aldrich). After allowing 9 minutes for patch perforation to take place, the E-head moved round the PatchPlate™ 48 wells at a time to obtain pre-compound hERG current measurements. The F-head then added 3.5 μl of solution from each well of the compound plate to 4 wells on the PatchPlate™ (the final DMSO concentration was 0.33% in every well). This was achieved by moving from the most dilute to the most concentrated well of the compound plate to minimise the impact of any compound carry-over. After approximately 3.5 mins incubation, the E-head then moved around all 384-wells of the PatchPlate™ to obtain post-compound HERG current measurements. In this way, non-cumulative concentration-effect curves could be produced where, providing the acceptance criteria were achieved in a sufficient percentage of wells (see below), the effect of each concentration of test compound was based on recording from between 1 and 4 cells.

The pre- and post-compound HERG current was evoked by a single voltage pulse consisting of a 20 s period holding at −70 mV, a 160 ms step to −60 mV (to obtain an estimate of leak), a 100 ms step back to −70 mV, a 1 s step to +40 mV, a 2 s step to −30 mV and finally a 500 ms step to −70 mV. In between the pre- and post-compound voltage pulses there was no clamping of the membrane potential. Currents were leak-subtracted based on the estimate of current evoked during the +10 mV step at the start of the voltage pulse protocol. Any voltage offsets in IonWorks™ HT were adjusted in one of two ways. When determining compound potency, a depolarising voltage ramp was applied to CHO-Kv1.5 cells and the voltage noted at which there was an inflection point in the current trace (i.e. the point at which channel activation was seen with a ramp protocol). The voltage at which this occurred had previously been determined using the same voltage command in conventional electrophysiology and found to be ˜15 mV (data not shown); thus an offset potential could be entered into the IonWorks™ HT software using this value as a reference point. When determining the basic electrophysiological properties of HERG, any offset was adjusted by determining the HERG tail current reversal potential in IonWorks™ HT, comparing it with that found in conventional electrophysiology (−82 mV; see FIG. 1c) and then making the necessary offset adjustment in the IonWorks™ HT software. The current signal was sampled at 2.5 kHz.

Pre- and post-scan HERG current magnitude was measured automatically from the leak subtracted traces by the IonWorks™ HT software by taking a 40 ms average of the current during the initial holding period at −70 mV (baseline current) and subtracting this from the peak of the tail current response. The acceptance criteria for the currents evoked in each well were: pre-scan seal resistance >60 MΩ, pre-scan HERG tail current amplitude >150 pA; post-scan seal resistance >60 MΩ. The degree of inhibition of the hERG current was assessed by dividing the post-scan hERG current by the respective pre-scan HERG current for each well.

### Methods of Preparation

The compounds of the present invention can be prepared in a number of ways well known to one skilled in the art of organic synthesis. The compounds of the present invention can be synthesized using the methods described below, together with synthetic methods known in the art of synthetic organic chemistry, or variations thereon as appreciated by those skilled in the art. Such methods include, but are not limited to, those described below. All references cited herein are hereby incorporated in their entirety by reference.

The novel compounds of this invention may be prepared using the reactions and techniques described herein. The reactions are performed in solvents appropriate to the reagents and materials employed and are suitable for the transformations being effected. Also, in the description of the synthetic methods described below, it is to be understood that all proposed reaction conditions, including choice of solvent, reaction atmosphere, reaction temperature, duration of the experiment and workup procedures, are chosen to be the conditions standard for that reaction, which should be readily recognized by one skilled in the art. It is understood by one skilled in the art of organic synthesis that the functionality present on various portions of the molecule must be compatible with the reagents and reactions proposed. Such restrictions to the substituents, which are not compatible with the reaction conditions, will be readily apparent to one skilled in the art and alternate methods must then be used.

The starting materials for the examples contained herein are either commercially available or are readily prepared by standard methods from known materials. For example the following reactions are illustrations but not limitations of the preparation of some of the starting materials and examples used herein.

General procedures for making the compounds of the invention is as follows:

The invention will now be illustrated by the following nonlimiting examples.
I. temperatures are given in degrees Celsius (° C.); unless otherwise stated, operations were carried out at room or ambient temperature, that is, at a temperature in the range of 18-25° C.;
II. organic solutions were dried over anhydrous magnesium sulfate; evaporation of solvent was carried out using a rotary evaporator under reduced pressure (600-4000 Pascals; 4.5-30 mm Hg) with a bath temperature of up to 60° C.;
III. chromatography means flash chromatography on silica gel; thin layer chromatography (TLC) was carried out on silica gel plates;
IV. in general, the course of reactions was followed by TLC or HPLC and reaction times are given for illustration only;
V. melting points are uncorrected and (dec) indicates decomposition;
VI. final products had satisfactory proton nuclear magnetic resonance (NMR) spectra;
VII. when given, NMR data is in the form of delta values for major diagnostic protons, given in parts per million (ppm) relative to tetramethylsilane (TMS) as an internal standard, determined at 300 MHz using deuterated chloroform (CDCl₃), dimethylsulphoxide (DMSO-d₆) or dimethylsulphoxide/TFA (DMSO-d₆/TFA-d) as solvent; conventional abbreviations for signal shape are used; for AB spectra the directly observed shifts are reported; coupling constants (J) are given in Hz;
VII. reduced pressures are given as absolute pressures in pascals (Pa); elevated pressures are given as gauge pressures in bars;
IX. non-aqueous reactions were run under a nitrogen atmosphere
X. solvent ratios are given in volume:volume (v/v) terms;
XI. Mass spectra (MS) were run using an automated system with atmospheric pressure chemical (APCI) or electrospray (+ES) ionization. Generally, only spectra where parent masses are observed are reported. The lowest mass major ion is reported for molecules where isotope splitting results in multiple mass spectral peaks (for example when chlorine is present).
XII. Commercial reagents were used without further purification.
XIII. 1-(3-bromo-4-chlorophenyl)ethanone was prepared according to Broxton et al, J. Chem. Soc. Perkin Trans., 1974, 1, 1769-1771.
XIV. Mass spectra were recorded using either a Hewlett Packard 5988A or a MicroMass Quattro-1 Mass Spectrometer and are reported as m/z for the parent molecular ion with its relative intensity.
XV. Room temperature refers to 20-25° C.
XVI. LC-MS HPLC conditions: Column: Agilent Zorbax SB-C8 2 mm ID×50 mm Flow: 1.4 mL/min Gradient: 95% A to 90% B over 3 min. hold 1 min ramp down to 95% A over 1 min and hold 1 min. Where A=2% acetonitrile in water with 0.1% formic acid and B=2% water in acetonitrile with 0.1% formic acid. UV-DAD 210-400 nm
XVII. Agilent preparative reverse phase HPLC conditions: Compounds were purified on a Phenomenex Luna C18 reverse phase column (250×21 mm, 10 micron particle size). To one skilled in the art, it is appreciated that the crude samples can be dissolved in methanol, DMF, or a wide range of acetonitrile/water mixtures with and without TFA, methanol, or DMF in concentrations ranging from dilute to concentrated. All purifications were run using 220 nm wavelength for collecting fractions. Retention time (t_{R})=min. Agilent Gradient 1 (AG1): 0% acetonitrile with 0.1% TFA 3 min, ramp 0-50% acetonitrile/water with 0.1% TFA over 12 min, hold at 50% acetonitrile/water for 3 min, 50-100% acetonitrile/water with 0.1% TFA over 7 min, flow rate of 40 ml/min. Agilent Gradient 2 (AG2): 10-100% acetonitrile/water with 0.1% TFA over 20 min, flow rate of 40 mL/min. Agilent Gradient 3 (AG3): 0% acetonitrile with 0.1% TFA 3 mins, ramp 0-100% acetonitrile/water with 0.1% TFA over 25 mins, flow rate of 40 ml/min.
XVIII. Normal phase chromatography conditions: Flash chromatography employed as a method for purification for selected intermediates. Isco CombiFlash Sq 16× instrument: pre-packaged disposable RediSep SiO₂ stationary phase columns (4, 12, 40, 120 gram sizes) with gradient elution at 5-125 mL/min of selected bi-solvent mixture, UV detection (190-760 nm range) or timed collection, 0.1 mm flow cell path length.
XIX. Microwave heating instrumentation: A Personal Chemistry Smith Synthesizer unit (monomodal, 2.45 GHz, 300 W max) was utilized for microwave heating of reactions.
XX. Terms and abbreviations: Solvent mixture compositions are given as volume percentages or volume ratios. In cases where the NMR spectra are complex; only diagnostic signals are reported. atm: atmospheric pressure; Boc: t-butoxycarbonyl; Cbz: benzyloxycarbonyl; DCM: dichloromethane; DIPEA: diisopropylethylamine; DMF: N;N-dimethyl formamide; DMSO: dimethyl sulfoxide; Et₂O: diethyl ether; EtOAc: ethyl acetate; h: hour(s); HPLC: high pressure liquid chromatography; minute(s): min.; NMP: 1-methyl-2-pyrrolidinone; NMR: nuclear magnetic resonance; psi: pounds per square inch; TFA: trifluoroacetic acid; THF: tetrahydrofuran; ACN: acetonitrile.

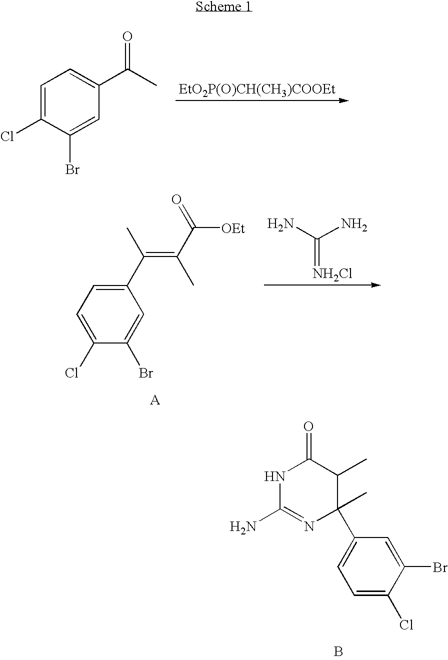

### EXAMPLE 1

### 2-Amino-6-(3-bromo-4-chlorophenyl)-5,6-dimethyl-5,6-dihydropyrimidin-4(3H)-one trifluoroacetate

### Scheme #1, B

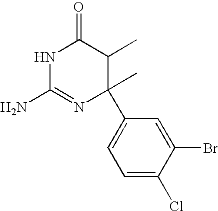

To a suspension of guanidine hydrochloride (0.138 g, 1.45 mmol) and sodium methoxide (0.087 g, 2.31 mmol) in NMP (2 mL) was added ethyl (2E)-3-(3-bromo-4-chlorophenyl)-2-methylbut-2-enoate (0.100 g, 0.29 mmol) and the reaction was subjected to microwaves at 200° C. for 15 min two times. The NMP was removed under reduced pressure and to the syrup was added acetonitrile: water:TFA (75:25:0.1, 2 mL) with a few drops of methanol. After the precipitates were removed, the filtrate was purified using RP-HPLC (AG1) (t_{R}=13.5 min). The combined purified fractions were lyophilized to give the title compound (6.1 mg, 5% yield). ¹H NMR (300 MHz, DMSO-d₆-/TFA-d) δ 0.99 (dd, J=14.8, 7.1 Hz, 3H), 1.56 (s, 1.8H), 1.80 (s, 1.2H), 3.26 (q, J=7.1 Hz, 0.4H), 3.39 (q, J=7.1 Hz, 0.6H), 7.35 (dd, J=8.6, 2.3 Hz, 0.4H), 7.54 (dd, J=8.5, 2.3 Hz, 0.6H), 7.70 (dd, J=8.5, 3.1 Hz, 1.4H), 7.97 (d, J=2.3 Hz, 0.6H) m/z (APCI+) M+1 (330); t_{R} 1.63 min.

### Ethyl (2E)-3-(3-bromo-4-chlorophenyl)-2-methylbut-2-enoate

### Scheme #1, A

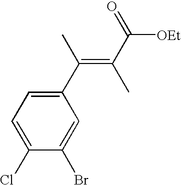

To a −78° C. stirred solution of triethyl 2-phosphonopropionate (2.10 mL, 9.42 mmol) in THF (10 mL) was added n-butyllithium in hexanes (2.5 M, 3.80 mL, 9.42 mmol) and the reaction stirred at −78° C. for 10 min. To this mixture was added 1-(3-bromo-4-chlorophenyl)ethanone (2.00 g, 8.57 mmol) and the reaction was allowed to warm up to room temperature. After 18 h the THF was removed under reduced pressure and the yellow solid was triturated with hexanes (50 mL) for 1 h. The mixture was filtered through Celite and the filtrate concentrated under reduced pressure to give a mixture of the E/Z-isomers of the title compound (2.40 g). LC two peaks 40:60 t_{R}=2.98:3.08 min. This mixture was used in the next reaction without further purifications.

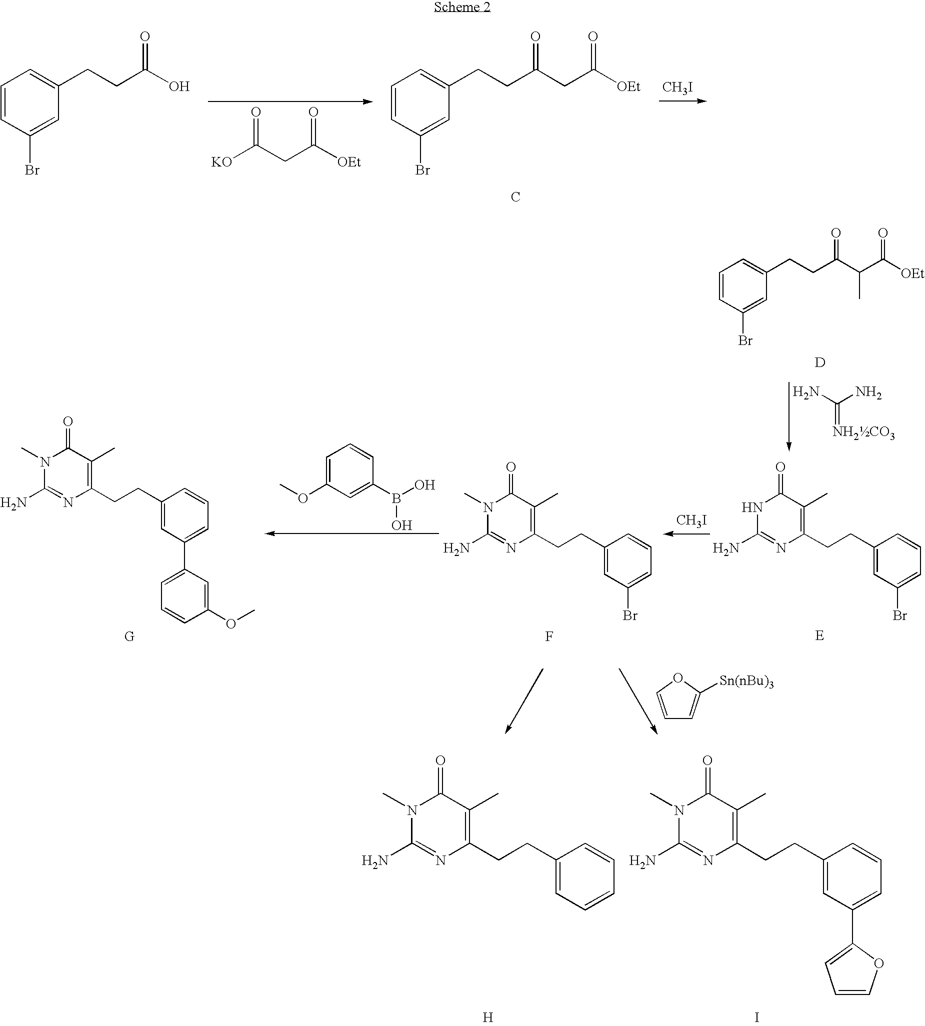

### EXAMPLE 2

### 2-Amino-6-[2-(3′-methoxybiphenyl-3-yl)ethyl]-3,5-dimethylpyrimidin-4(3H)-one trifluoroacetate

### Scheme #2, G

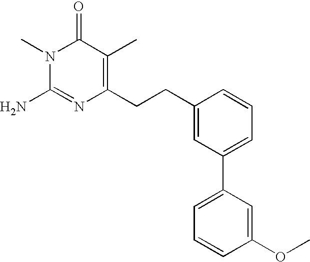

To 2-amino-6-[2-(3-bromophenyl)ethyl]-3,5-dimethylpyrimidin-4(3H)-one (100.0 mg, 0.310 mmol) was added cesium carbonate (303.0 mg, 0.931 mmol), 3-methoxyphenylboronic acid (61.0 mg, 0.403 mmol), dichlorobis(triphenylphosphine) palladium(II) (11.0 mg, 0.0155 mmol), and 2.0 mL 7:3:2 1,2-dimethoxyethane: water: ethanol. The reaction was subjected to microwaves for 15 minutes at 150° C. after which the aqueous layer was removed and the organic solvents removed under reduced pressure. To the resulting brown gum was added acetonitrile: water:TFA (75:25:0.1, 2.0 mL) and the formed precipitate was removed. The filtrate was purified using RP-HPLC AG2 (t_{R}=14.2 min). The combined purified fractions Were lyophilized to give the title compound (47.3 mg, 32% yield). ¹H NMR (300 MHz, DMSO-d₆/TFA-d) δ 1.79 (s, 3H), 2.83-2.97 (m, 4H), 3.34 (s, 3H), 3.83 (s, 3H), 6.95 (d, J=9.9 Hz, 1H), 7.16 (t, J=2.1 Hz, 1H), 7.21 (d, J=7.7 Hz, 1H), 7.30 (d, J=7.7 Hz, 1H), 7.34-7.43 (m, 2H), 7.53 (d, J=7.8 Hz, 1H), 7.59 (s, 1H); m/z (APCI+) M+1 (350); t_{R}=2.07 min.

### EXAMPLE 3

### 2-Amino-3,5-dimethyl-6-(2-phenylethyl)pyrimidin-4(3H)-one trifluoroacetate

### Scheme #2, H

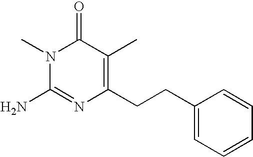

To a solution 2-amino-6-[2-(3-bromophenyl)ethyl]-3,5-dimethylpyrimidin-4(3H)-one (100.0 mg, 0.310 mmol) in 20 mL methanol was added 10% palladium on carbon (approximately 15 mg) and the reaction was charged with 50 PSI hydrogen. After shaking on a Parr Shaker for 1 h, the catalyst was removed by filtration and the solvent removed from the filtrate under reduced pressure. The resulting residue was dissolved in acetonitrile (2.0 mL) with a few drops of water and purified using RP-HPLC AG2 (t_{R}=6.7 min). The combined purified fractions were lyophilized to give the title compound (26.6 mg, 24% yield). ¹H NMR (300 MHz, DMSO-d₆/TFA-d) δ 1.75 (s, 3H), 2.77-2.90 (m, 4H), 3.34 (s, 3H), 7.21-7.34 (m, 5H), m/z (APCI+) M+1 (244), t_{R}=1.55 min.

### EXAMPLE 4

### 2-Amino-6-{2-[3-(2-furyl)phenyl]ethyl}-3,5-dimethylpyrimidin-4(3H)-one trifluoroacetate

### Scheme #2, I

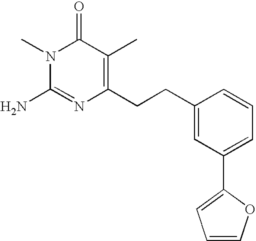

To 2-amino-6-[2-(3-bromophenyl)ethyl]-3,5-dimethylpyrimidin-4(3H)-one (100.0 mg, 0.310 mmol) in 2.0 mL 1,2-dimethoxyethane: water:ethanol (7:3:2) was added cesium carbonate (303.0 mg, 0.931 mmol), dichlorobis(triphenylphosphine)palladium(II) (11.0 mg, 0.0155 mmol), and 2-(Tributylstannyl)furan (0.293 mL, 0.931 mmol). The reaction was subjected to microwaves for 15 minutes at 150° C. after which the aqueous layer was removed and the organic solvents removed under reduced pressure. To the resulting brown oil was added acetonitrile (2.0 mL) with a few drops water and the formed precipitate was removed. The filtrate was purified using RP-HPLC AG2 (t_{R}=9.1 min). The combined purified fractions were lyophilized to give the title compound (30.6 mg, 23% yield). ¹H NMR (300 MHz, DMSO-d₆/TFA-d) δ 1.81 (s, 3H), 2.83-2.93 (m, 4H), 3.34 (s, 3H), 6.59 (dd, J=3.4, 1.8 Hz, 1H), 6.90 (d, J=3.3 Hz, 1H), 7.22 (d, J=7.8 Hz, 1H), 7.36 (t, J=7.7 Hz, 1H), 7.58 (d, J=7.8 Hz, 1H), 7.65 (s, 1H), 7.72 (d, J=1.1 Hz, 1H), m/z (APCI+) M+1 (244); t_{R}=1.98 min.

### EXAMPLE 5

### 2-Amino-6-[2-(3-bromophenyl)ethyl]-3,5-dimethylpyrimidin-4(3H)-one trifluoroacetate

### Scheme #2, F

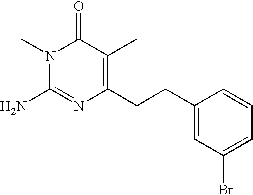

To a suspension of 2-amino-6-[2-(3-bromophenyl)ethyl]-5-methylpyrimidin-4(3H)-one (573.0 mg, 1.86 mmol) in ethanol (10 mL) was added powdered potassium hydroxide (188.0 mg, 3.35 mmol). Once homogeneous, methyl iodide (0.417 mL, 6.69 mmol) was added and the reaction heated at reflux. After 6 h the solvent was removed under reduced pressure and the resulting solids stored under high vacuum for 18 h. Water (50 mL) was added and the solids triturated for 2 h. The precipitate was filtered and put under high vacuum at 50° C. resulting in a crude white powder (520 mg, 87% yield). The bulk of material was carried forward as is, while 100 mg was dissolved in acetonitrile/water and purified by RP-HPLC AG2 (t_{R}=8.5 min). The combined purified fractions were lyophilized to give the title compound (46.7 mg, 29% yield). ¹H NMR (300 MHz, DMSO-d₆/TFA-d) δ 1.80 (s, 3H), 2.77-2.89 (m, 4H), 3.34 (s, 3H), 7.24-7.33 (m, 2H), 7.43 (d, J=7.5 Hz, 1H), 7.57 (s, 1H), m/z (APCI+) M+1 (322); t_{R}=1.78 min.

### EXAMPLE 6

### 2-Amino-6-[2-(3-bromophenyl)ethyl]-5-methylpyrimidin-4(3H)-one

### Scheme #2, E

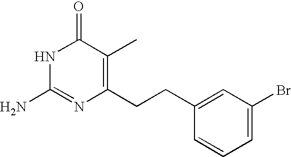

To the crude syrup of ethyl 5-(3-bromophenyl)-2-methyl-3-oxopentanoate was added ethanol (10.0 mL) and guanidine carbonate (1.20 g, 6.69 mmol), and the reaction was refluxed. After 4 h, the organic solvent was removed under reduced pressure and the resulting solids put under high vacuum at 50° C. The crude material was crystallized from water/ethanol and the crystals placed under high vacuum at 50° C. to give the title compound (607 mg, 59% yield). ¹H NMR (300 MHz, DMSO-d₆/TFA-d) δ 1.73 (s, 3H), 2.75-2.89 (m, 4H), 7.24-7.32 (m, 2H), 7.43 (dd, J=5.3, 1.9 Hz, 1H), 7.55 (s, 1H), m/z (APCI+) M+1 (308); t_{R}=1.70 min.

### Ethyl 5-(3-bromophenyl)-2-methyl-3-oxopentanoate

### Scheme #2, D

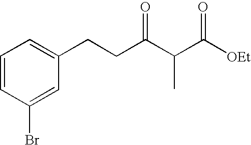

To ethyl 5-(3-bromophenyl)-3-oxopentanoate (1.00 g, 3.34 mmol) in a J-Kem tube was added THF (10.0 mL) and potassium tert-butoxide in THF (1.0 M, 4.01 mL, 4.01 mmol). After stirring for 10 min methyl iodide (0.31 mL, 5.01 mmol) was added and the reaction was stirred at room temperature for 1 h 45 min. The reaction was quenched with hydrochloric acid (1M, 4.0 mL) followed by 2.0 mL saturated aqueous sodium chloride. After stirring 20 min, the aqueous layer was removed and the organic solvent was removed on a Genevac evaporator to yield the crude title compound which was used in the next step without further purifications. m/z (+ES) M+1 (313); t_{R}=2.42 min.

### Ethyl 5-(3-bromophenyl)-3-oxopentanoate

### Scheme #2, C

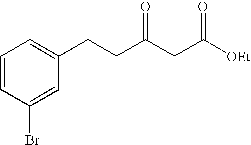

To magnesium chloride (10.39 g, 109.14 mmol) and potassium ethyl malonate (15.60 g, 91.68 mmol) in acetonitrile (600 mL) was added triethylamine (19.5 mL, 139.70 mmol). In a separate vessel, to 3-(3-bromophenyl)propanoic acid (10.00 g, 43.66 mmol) in 150 mL acetonitrile was added 1,1′-carbonyldiimidazole (7.79 g, 48.02 mmol). After stirreding for 2.5 h the mixture was transferred to an addition funnel and was added dropwise to the malonate reaction. After stirring for 18 h at room temperature, the reaction was heated at reflux for 3 h and cooled to room temperature. The solids were filtered and the filtrate evaporated under reduced pressure. The solids were partitioned between ethyl acetate and hydrochloric acid (1M). To the organic layer was added the previously stripped filtrate and the organic layer washed with hydrochloric acid (1M) and brine, dried over magnesium sulfate, and the solvent removed under reduced pressure. The resulting oil was redissolved in ethyl acetate and washed three times with a 4:1 dilution of water to saturated sodium bicarbonate solution, once with hydrochloric acid (1M) and once with brine, dried over magnesium sulfate, and the solvent removed under reduced pressure to yield the title compound (13.19 g, 100.8% yield). ¹H NMR (300 MHz, DMSO-d₆/TFA-d) δ 1.17 (t, J=7.1 Hz, 3H), 2.75-2.92 (m, 4H), 3.60 (s, 2H), 4.08 (q, J=7.1 Hz, 2H), 7.20-7.27 (m, 2H), 7.36-7.40 (m, 1H), 7.43 (s, 1H), m/z (+ES) M+1 (299); t_{R}=2.29 min.

Compounds below were prepared according to scheme #2 using benzyl bromide in place of methyl iodide. Example 8 was used in the preparation of Example 7 using the conditions found in Example 2.

### EXAMPLE 7

### 2-Amino-5-benzyl-6-[2-(3′-methoxybiphenyl-3-yl)ethyl]-3-methylpyrimidin-4(3H)-one trifluoroacetate

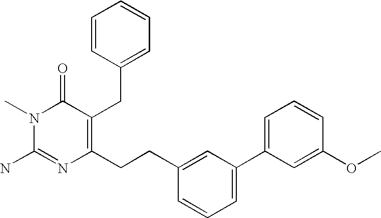

¹H NMR (300 MHz, DMSO-d₆/TFA-d) δ one aromatic proton is missing 2.72-2.80 (m, 2H), 2.83-2.91 (m, 2H), 3.37 (s, 3H), 3.75 (s, 2H), 3.84 (s, 3H), 6.95 (dd, J=8.2, 2.0 Hz, 1H), 7.14-7.30 (m, 7H), 7.35-7.41 (m, 2H), 7.46 (s, 1H), 7.49-7.54 (m, 1H), m/z (APCI+) M+1 (426); t_{R}=2.41 min.

### EXAMPLE 8

### 2-Amino-5-benzyl-6-[2-(3-bromophenyl)ethyl]-3-methylpyrimidin-4(3H)-one trifluoroacetate

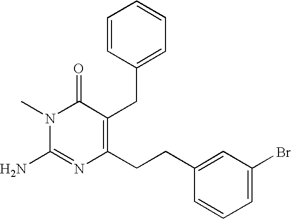

¹H NMR (300 MHz, DMSO-d₆/TFA-d) δ 2.64-2.72 (m, 2H), 2.76-2.83 (m, 2H), 3.36 (s, 3H), 3.75 (s, 2H), 7.17-7.31 (m, 7H), 7.39-7.46 (m, 2H), m/z (APCI+) M+1 (398); t_{R}=2.20 min.

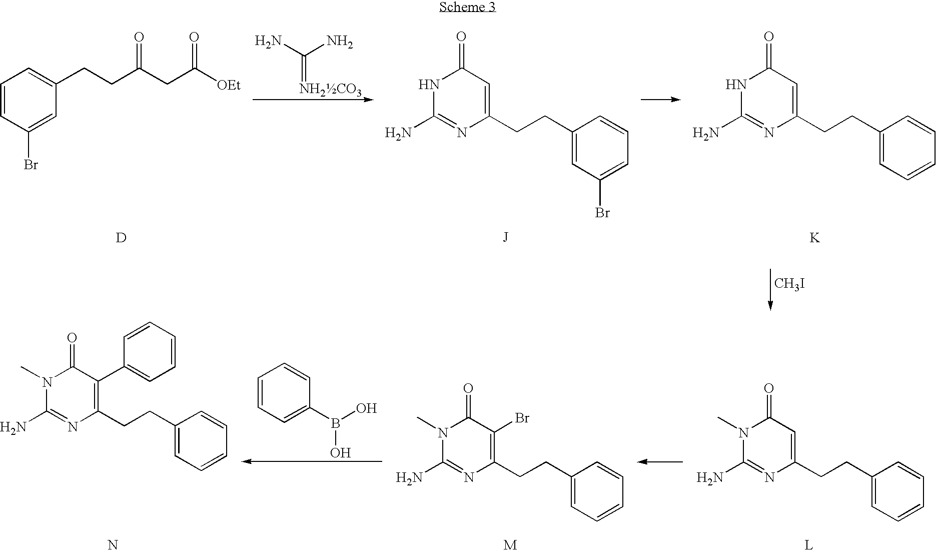

### EXAMPLE 9

### 2-Amino-3-methyl-5-phenyl-6-(2-phenylethyl)pyrimidin-4(3H)-one trifluoroacetate

### Scheme #3, N

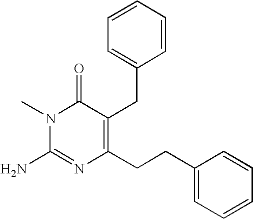

To 2-amino-5-bromo-3-methyl-6-(2-phenylethyl)pyrimidin-4(3H)-one (100.0 mg, 0.324 mmol) was added cesium carbonate (317.0 mg, 0.973 mmol), 3-methoxyphenylboronic acid (51.0 mg, 0.422 mmol), dichlorobis(triphenylphosphine) palladium(II) (12.0 mg, 0.0162 mmol), and 1,2-dimethoxyethane: water: ethanol (2.0 mL, 7:3:2). The reaction was subjected to microwaves for 15 min at 150° C. after which the aqueous layer was removed and the organic solvents removed under reduced pressure. To the resulting brown gum was added DMF: acetonitrile: water (2.0 mL) and the formed precipitate removed. The filtrate was purified using RP-HPLC AG2 (t_{R}=8.94 min). The combined purified fractions were lyophilized to give the title compound (44.0 mg, 32% yield). ¹H NMR (300 MHz, DMSO-d₆/TFA-d) δ 2.58-2.63 (m, 2H), 2.80-2.85 (m, 2H), 3.38 (s, 3H), 7.02-7.12 (m, 4H), 7.19-7.28 (m, 3H), 7.38-7.43 (m, 3H), m/z (APCI+) M+1 (306); t_{R}=1.95 min.

### EXAMPLE 10

### 2-Amino-5-bromo-3-methyl-6-(2-phenylethyl)pyrimidin-4(3H)-one

### Scheme #3, M

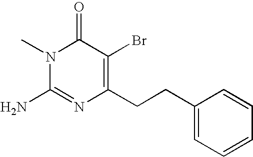

To an ice bath cooled suspension of 2-amino-3-methyl-6-(2-phenylethyl)pyrimidin-4(3H)-one (900 mg, 3.92 mmol) in DMF was added N-bromosuccinimide (765 mg, 4.32 mmol) and reaction was warmed to room temperature. After 10 min the reaction was diluted with 250 mL water and the white precipitate was removed by filtration and dried under high vacuum at 50° C. over night to give the title compound (980 mg, 81% yield). ¹H NMR (300 MHz, DMSO-d₆/TFA-d) δ 2.87-2.98 (m, 4H), 3.38 (s, 3H), 7.22-7.35 (m, 5H), m/z (APCI+) M+1 (308); t_{R}=2.01 min.

### 2-Amino-3-methyl-6-(2-phenylethyl)pyrimidin-4(3H)-one

### Scheme #3, L

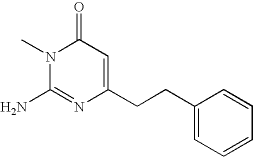

To a suspension of 2-amino-6-(2-phenylethyl)pyrimidin-4(3H)-one (1.76 g, 5.94 mmol) in ethanol (15 mL) was added powdered potassium hydroxide (0.934 g, 16.64 mmol). Once homogeneous, methyl iodide (1.33 mL, 21.39 mmol) was added and the reaction heated at reflux. After 2.5 h the solvent was removed under reduced pressure and the resulting solids dried under high vacuum. After triturating with water (50 mL) for 2 h, the precipitate was filtered and dried under high vacuum at 50° C. for 18 h to give the title compound (1.110 g, 80% yield). ¹H NMR (300 MHz, DMSO-d₆/TFA-d) δ 2.75-2.83 (m, 2H), 2.87-2.99 (m, 2H), 3.29 (s, 3H), 5.91 (s, 1H), 7.17-7.38 (m, 5H), m/z (APCI+) M+1 (230); t_{R}=1.32 min.

### 2-Amino-6-(2-phenylethyl)pyrimidin-4(3H)-one

### Scheme #3, K

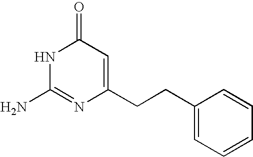

To a suspension of 2-amino-6-[2-(3-bromophenyl)ethyl]pyrimidin-4(3H)-one (1.75 g, 5.95 mmol) in methanol was added 10% palladium on carbon (175 mg, 0.164 mmol) and vessel charged with 50 PSI hydrogen. After shaking on a Parr Shaker for 1.5 h the catalyst was filtered and the solvent removed under reduced pressure to give the title compound which was carried forward as is into the next reaction without purification. m/z (APCI+) M+1 (216); t_{R}=1.25 min.

### 2-Amino-6-[2-(3-bromophenyl)ethyl]pyrimidin-4(3H)-one

### Scheme #3, J

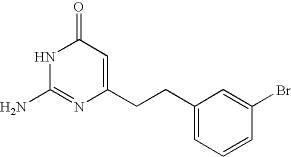

Ethyl 5-(3-bromophenyl)-3-oxopentanoate (1.00 g, 3.34 mmol each) was added to 3 vessels followed by addition of ethanol (10.0 mL) and guanidine carbonate (1.20 g, 6.69 mmol) into each reaction and the reactions were refluxed for 4 h. The reactions were combined and the organic solvent removed under reduced pressure. The resulting white solid was dried under high vacuum at 50° C. The crude material was crystallized from water/ethanol and the crystals dried under high vacuum at 50° C. to give the title compound (1.75 g, 59% yield). ¹H NMR (300 MHz, DMSO-d₆/TFA-d) δ 2.75-2.82 (m, 2H), 2.89-2.94 (m, 2H), 5.82 (s, 1H), 7.27-7.31 (m, 2H), 7.41-7.44 (m, 1H), 7.53 (s, 1H), m/z (APCI+) M+1 (294); t_{R}=1.39 min.

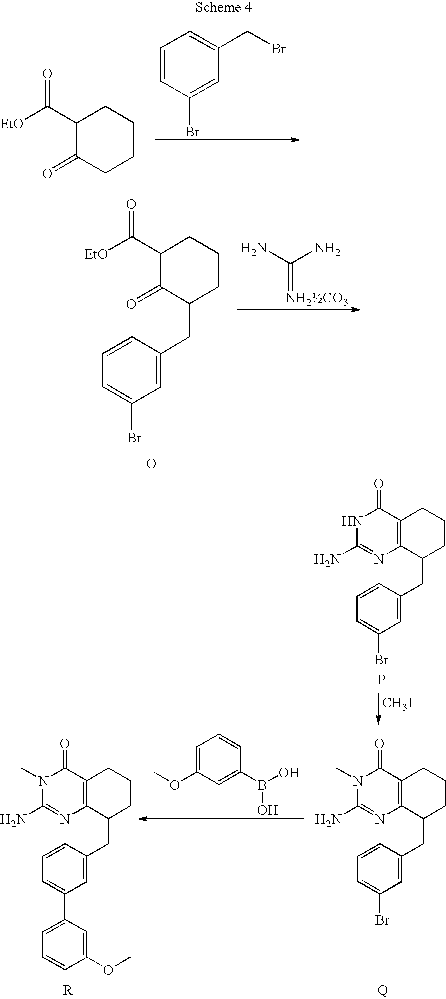

### EXAMPLE 14

### 2-Amino-8-[(3′-methoxybiphenyl-3-yl)methyl]-3-methyl-5,6,7,8-tetrahydroquinazolin-4(3H)-one trifluoroacetate

### Scheme #4, R

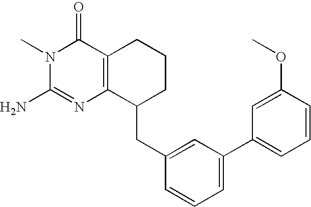

To crude 2-amino-8-(3-bromobenzyl)-3-methyl-5,6,7,8-tetrahydroquinazolin-4(3H)-one (100.0 mg, 0.299 mmol) was added cesium carbonate (283.0 mg, 0.870 mmol), 3-methoxyphenylboronic acid (57.0 mg, 0.377 mmol), dichlorobis(triphenylphosphine) palladium(II) (10.0 mg, 0.0145 mmol), and 1,2-dimethoxyethane: water: ethanol (2.0 mL, 7:3:2). The reaction was subjected to microwaves for 15 min at 150° C. after which the aqueous layer was removed and the organic solvents evaporated under reduced pressure. To the resulting brown gum was added DMF, the formed precipitate was removed, and the filtrate was purified using RP-HPLC AG2 (t_{R}=11.4 min). The combined purified fractions were lyophilized to give the title compound (41.6 mg, 28% yield). ¹H NMR (300 MHz, DMSO-d₆/TFA-d) δ 1.43-1.81 (m, 4H), 2.16-2.27 (m, 1H), 2.37-2.48 (m, 1H), 2.70 (t, J=12.9 Hz, 1H), 2.87-2.98 (m, 1H), 3.15-3.24 (m, 1H), 3.36 (s, 3H), 3.84 (s, 3H), 6.96 (dd, J=8.1, 2.0 Hz, 1H), 7.20-7.25 (m, 2H), 7.35-7.45 (m, 3H), 7.54-7.65 (m, 2H), m/z (APCI+) M+1 (376); t_{R}=2.20 min.

### EXAMPLE 15

### 2-Amino-8-(3-bromobenzyl)-3-methyl-5,6,7,8-tetrahydroquinazolin-4(3H)-one trifluoroacetate

### Scheme #4, Q

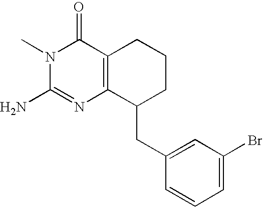

To crude 2-amino-8-(3-bromobenzyl)-5,6,7,8-tetrahydroquinazolin-4(3H)-one (100.0 mg, 0.299 mmol) in ethanol (2.0 mL) was added powdered potassium hydroxide (30.0 mg, 0.539 mmol). Once homogeneous, methyl iodide (0.067 mL, 1.077 mmol) was added and the reaction heated at reflux. After 4 h the solvent was removed under reduced pressure and the resulting solids dried under high vacuum. This material was triturated with water (5 mL) for 2 h and the precipitate filtered off and used as is in preparing Example 14. A second batch of material was prepared using exactly the same procedure as described above with the only difference being the reaction was on three times the scale. The resulting precipitate was further purified using RP-HPLC AG2 (t_{R}=10.4 min). The combined purified fractions were lyophilized to give the title compound as a TFA salt (175 mg, 42% yield). ¹H NMR (300 MHz, DMSO-d₆/TFA-d) δ1.46-1.51 (m, 2H), 1.64-1.82 (m, 2H), 2.14-2.25 (m, 1H), 2.41 (d, J=17.3 Hz, 1H), 2.62 (t, J=12.9 Hz, 1H), 2.85 (d, J=11.1 Hz, 1H), 3.10 (dd, J=13.2, 2.7 Hz, 1H), 3.35 (s, 3H), 7.29 (t, J=7.7 Hz, 1H), 7.37 (d, J=7.6 Hz, 1H), 7.45 (d, J=7.7 Hz, 1H), 7.69 (s, 1H); m/z (APCI+) M+1 (348); t_{R}=1.96 min.

### EXAMPLE 16

### 2-Amino-8-(3-bromobenzyl)-5,6,7,8-tetrahydroquinazolin-4(3H)-one trifluoroacetate

### Scheme #4, P

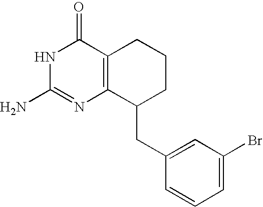

To crude ethyl 3-(3-bromobenzyl)-2-oxocyclohexanecarboxylate (626.0 mg, 1.85 mmol) was added ethanol (10.0 mL) and guanidine carbonate (332.0 mg, 1.85 mmol) and the mixture refluxed for 1 h. The organic solvent was removed under reduced pressure and the resulting solids triturated with water (20 mL). The precipitate was filtered and dried under high vacuum at 50° C. to give 607 mg of the crude title compound. A portion of this (100 mg) was dissolved in acetonitrile/water and purified using RP-HPLC AG1 (t_{R}=15.4 min). The combined purified fractions were lyophilized to give the title compound (37.4 mg). ¹H NMR (300 MHz, DMSO-d₆/TFA-d) δ 1.47-1.50 (m, 2H), 1.61-1.79 (m, 2H), 2.10-2.21 (m, 1H), 2.32-2.42 (m, 1H), 2.62 (t, J=12.0 Hz, 1H), 2.77-2.88 (m, 1H), 3.09 (dd, J=13.3, 2.8 Hz, 1H), 7.32 (quintet, J=7.8 Hz, 2H), 7.45 (d, J=7.7 Hz, 1H), 7.66 (s, 1H), m/z (APCI+) M+1 (334); t_{R}=1.91 min.

### Ethyl 3-(3-bromobenzyl)-2-oxocyclohexanecarboxylate

### Scheme #4, O

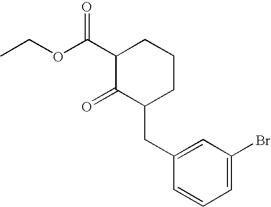

To ethyl 2-oxocyclohexanecarboxylate (0.470 mL, 2.94 mmol), which was previously dried over 4 Å molecular sieves 18 h, was added THF (10 mL) and this solution was cooled in a dry ice/acetone bath. Lithium diisopropylamide mono(tetrahydrofuran) 1.5 M in cyclohexane (4.11 mL, 6.17 mmol) was added and the reaction stirred 10 min then warmed in a water/ice bath and stirred for 15 min. To this was added a solution of 1-bromo-3-(bromomethyl)benzene (881 mg, 3.53 mmol) in THF (2.0 mL) and the reaction was warmed to room temperature. After 5 h the reaction was quenched with water and partitioned between ethyl acetate/hydrochloric acid (1M) and the organic layer washed twice with hydrochloric acid (1M) and once with brine, and dried over sodium sulfate. The solvent was removed under reduced pressure to give the crude title compound which was used in the next reaction without further purifications.

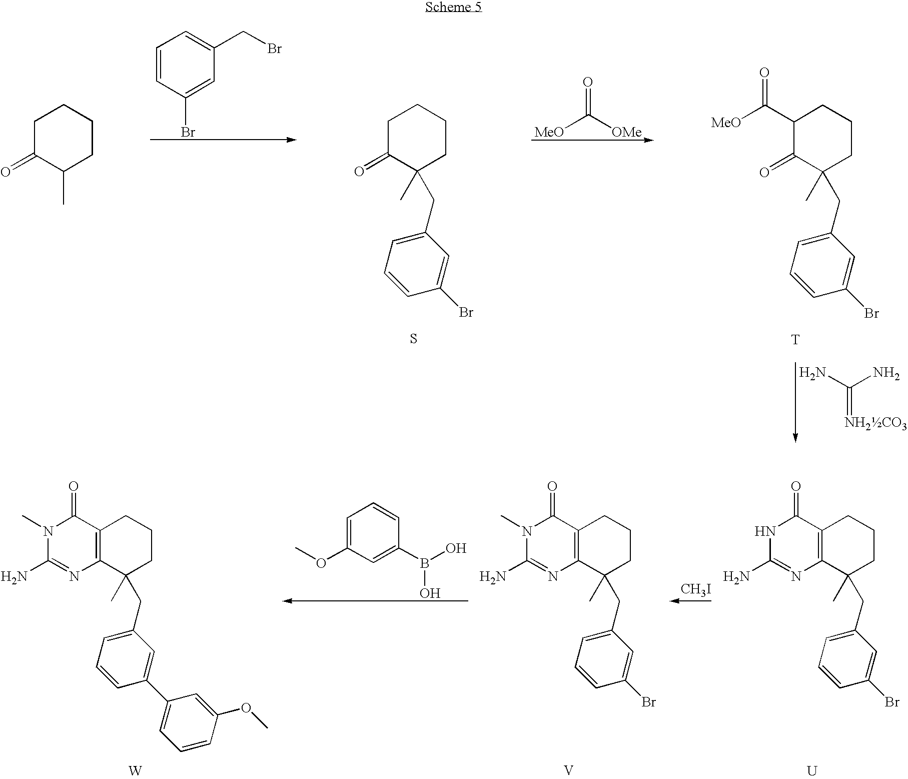

### EXAMPLE 17

### 2-Amino-8-[(3′-methoxybiphenyl-3-yl)methyl]-3,8-dimethyl-5,6,7,8-tetrahydroquinazolin-4(3H)-one trifluoroacetate

### Scheme #5, W

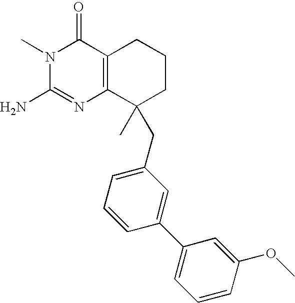

To 2-amino-8-(3-bromobenzyl)-3,8-dimethyl-5,6,7,8-tetrahydroquinazolin-4(3H)-one (100.0 mg, 0.276 mmol) was added cesium carbonate (270.0 mg, 0.828 mmol), 3-methoxyphenylboronic acid (55.0 mg, 0.359 mmol), dichlorobis(triphenylphosphine) palladium(II) (10.0 mg, 0.0138 mmol), and 1,2-dimethoxyethane: water: ethanol (2.0 mL, 7:3:2). The reaction was subjected to microwaves for 15 min at 150° C. after which the aqueous layer was removed and the organic solvents evaporated under reduced pressure. To the residue was added acetonitrile, the formed precipitates were removed, and the filtrate was purified using RP-HPLC AG2 (t_{R}=11.1 min). The combined purified fractions were lyophilized to give the title compound (40.7 mg, 29% yield). ¹H NMR (300 MHz, DMSO-d₆/TFA-d) δ 1.27 (s, 3H), 1.31-1.43 (m, 1H), 1.56-1.86 (m, 3H), 2.27 (t, J=5.5 Hz, 2H), 3.05 (s, 2H), 3.36 (s, 3H), 3.83 (s, 3H), 6.95 (dd, J=8.2, 1.9 Hz, 1H), 7.13-7.22 (m, 2H), 7.39 (dd, J=14.3, 7.7 Hz, 2H), 7.48 (s, 1H), 7.53-7.68 (m, 2H), m/z (APCI+) M+1 (390); t_{R}=2.27 min.

### EXAMPLE 18

### 2-Amino-8-(3-bromobenzyl)-3,8-dimethyl-5,6,7,8-tetrahydroquinazolin-4(3H)-one

### Scheme #5, V

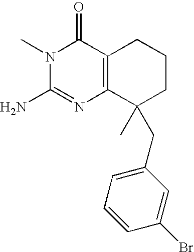

To 2-amino-8-(3-bromobenzyl)-8-methyl-5,6,7,8-tetrahydroquinazolin-4(3H)-one (1.768 g, 5.08 mmol) in ethanol (10.0 mL) was added powdered potassium hydroxide (0.512 g, 9.14 mmol). Once homogeneous, methyl iodide (1.14 mL, 18.28 mmol) was added and the reaction heated at reflux. After 3.5 h the solvent was removed under reduced pressure and the resulting solids placed under high vacuum. After triturating with water (50 mL) for 1 h, the white precipitate was filtered and dried under high vacuum at 50° C. for 18 h to give the title compound (1.737 g, 94% yield). ¹H NMR (300 MHz, DMSO-d₆/TFA-d) δ 1.20 (s, 3H), 1.28-1.40 (m, 1H), 1.58-1.78 (m, 3H), 2.27 (t, J=6.0 Hz, 2H), 2.96 (d, J=9.2 Hz, 2H), 3.35 (s, 3H), 7.25 (dt, J=16.0, 7.9 Hz, 2H), 7.46 (d, J=6.1 Hz, 2H), m/z (APCI+) M+1 (362); t_{R}=2.07 min.

### EXAMPLE 19

### 2-Amino-8-(3-bromobenzyl)-8-methyl-5,6,7,8-tetrahydroquinazolin-4(3H)-one

### Scheme #5, U

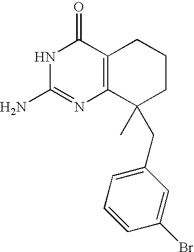

To methyl 3-(3-bromobenzyl)-3-methyl-2-oxocyclohexanecarboxylate (1.86 g, 5.48 mmol) was added ethanol (20.0 mL), guanidine carbonate (0.987 g, 5.48 mmol), and the reaction was refluxed. After 18 h the organic solvent was removed under reduced pressure and the resulting solids triturated with water (50 mL). The white precipitate was filtered and dried under high vacuum at 50° C. to give the title compound (1.85 g, 97% yield). ¹H NMR (300 MHz, DMSO-d₆/TFA-d) δ 1.21 (s, 3H), 1.31-1.41 (m, 1H), 1.57-1.77 (m, 3H), 2.18-2.27 (m, 2H), 2.95 (s, 2H), 7.41-7.50 (m, 2H), 7.19-7.31 (m, 2H), m/z (APCI+) M+1 (348); t_{R}=1.91 min.

### Methyl 3-(3-bromobenzyl)-3-methyl-2-oxocyclohexanecarboxylate

### Scheme #5, T

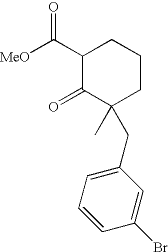

To hexanes washed 60% sodium hydride in mineral oil (706 mg, 17.65 mmol) was added dioxane (15 mL) and dimethyl carbonate (3.0 mL, 35.29 mmol). The reaction was heated in a 90° C. bath, a solution of 2-(3-bromobenzyl)-2-methylcyclohexanone (1.54 g, 7.06 mmol) in dioxane (10 mL) was added dropwise over 1.5 h and the reaction was heated at reflux. After 1.5 h the reaction was cooled in an ice bath and quenched with methanol. The reaction was partitioned between ethyl acetate/hydrochloric acid (1M) and the organic layer washed twice with hydrochloric acid (1M) and once with brine, dried over magnesium sulfate, and the solvents were removed under reduced pressure. The crude title compound (1.86 g) was dried under high vacuum and was used without further purifications in the next reaction. m/z (ES+) M+1 (339); t_{R}=2.76 min.

### 2-(3-Bromobenzyl)-2-methylcyclohexanone

### Scheme #5, S

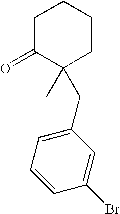

To a −30° C. cooled solution of 2-methylcyclohexanone (1.00 g, 8.92 mmol) in THF (40 mL) was added potassium tertbutoxide 1.0M in THF (8.92 mL, 8.92 mmol) and the bath temperature was maintained between −20 to −30° C. After 30 min the reaction was cooled in a −78° C. bath and 1-bromo-3-(bromomethyl)benzene (2.23 g, 8.92 mmol) was added. The reaction was allowed to warm up to room temperature as the bath warmed over night. After a total of 18 h, the reaction was quenched with water (1 mL) and the solvent removed under reduced pressure. The crude oil was partitioned between ethyl acetate/hydrochloric acid (1M) and the organic layer washed two times with hydrochloric acid (1M) and once with brine, dried over magnesium sulfate, and the solvent removed under reduced pressure. The crude oil was purified on silica gel (50 g) eluting with 40% ethyl acetate in hexanes. The solvent was removed from the combined purified fractions under reduced pressure to yield the title compound (1.54 g, 61% yield). ¹H NMR (300 MHz, DMSO-d₆/TFA-d) δ 0.94 (s, 3H), 1.44-1.53 (m, 1H), 1.63-1.89 (m, 5H), 2.34-2.43 (m, 1H), 2.54-2.62 (m, 1H), 2.77 (d, J=13.4 Hz, 1H), 2.95 (d, J=13.4 Hz, 1H), 7.12 (d, J=7.7 Hz, 1H), 7.23 (t, J=7.8 Hz, 1H), 7.33 (s, 1H), 7.40 (d, J=7.9 Hz, 1H), t_{R}=2.59 min.

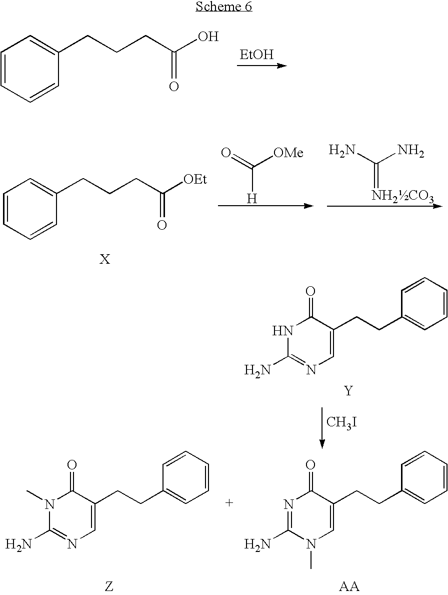

### EXAMPLE 20

### 2-Amino-3-methyl-5-(2-phenylethyl)pyrimidin-4(3H)-one trifluoroacetate

### Scheme #6, Z

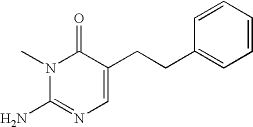

To 2-amino-5-(2-phenylethyl)pyrimidin-4(3H)-one trifluoroacetate (99.7 mg, 0.303 mmol) in ethanol (3.0 mL) was added powdered potassium hydroxide (51 mg, 0.908 mmol). Once homogeneous, methyl iodide (75 uL, 1.21 mmol) was added and the reaction sealed and heated at 80° C. After 4 h the solvent was removed under reduced pressure, the resulting solids dissolved in acetonitrile: water:TFA (75:25:0.1, 3 mL) and purified using RP-HPLC AG1 (t_{R}=11.7 min). The combined purified fractions were lyophilized to give the title compound (27 mg, 26% yield). ¹H NMR (300 MHz, DMSO-d₆/TFA-d) δ 2.61 (t, J=7.7 Hz, 2H), 2.79 (t, J=7.3 Hz, 2H), 3.35 (s, 3H), 7.25 (dd, J=20.5, 6.7 Hz, 5H), 7.50 (s, 1H), m/z (ES+) M+1 (230); t_{R}=2.76 min.

### EXAMPLE 21

### 2-Amino-1-methyl-5-(2-phenylethyl)pyrimidin-4(1H)-one trifluoroacetate

### Scheme #6, AA

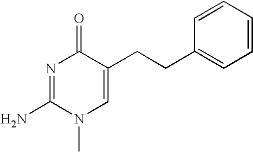

This was a by-product isolated by RP-HPLC AG1 (t_{R}=10.7 min) from the methylation of 2-amino-5-(2-phenylethyl)pyrimidin-4(3H)-one to give the title compound (23 mg, 22% yield). ¹H NMR (300 MHz, DMSO-d₆/TFA-d) δ 2.48-2.60 (m, 2H), 2.76-2.82 under DMSO (m, 2H), 3.45 (s, 3H), 7.18-7.23 (m, 3H), 7.28-7.33 (m, 2H), 7.69 (s, 1H), m/z (ES+) M+1 (230); t_{R}=3.39 min.

### EXAMPLE 22

### 2-Amino-5-(2-phenylethyl)pyrimidin-4(3H)-one trifluoroacetate

### Scheme #6, Y

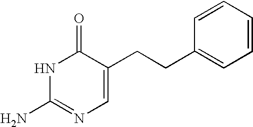

To ethyl 4-phenylbutanoate (100 mg, 0.52 mmol) in THF (1.0 mL) at −78° C. was added lithium diisopropylamide mono(tetrahydrofuran) 1.5 M in cyclohexane (0.38 mL, 0.57 mmol). After 10 min methyl formate (35 uL, 0.57 mmol) was added and reaction stirred for 5 min and then warmed up to room temperature. After 30 min the reaction was quenched with ethanol (3.0 mL) and guanidine carbonate (206 mg, 1.14 mmol) was added. The reaction was heated at reflux for 30 min after which the solvents were removed under reduced pressure. The resulting solids were dissolved in acetonitrile: water:TFA (75:25:0.1, 2.0 mL) and purified using RP-HPLC AG1 (t_{R}=10.9 min). The combined purified fractions were lyophilized to give the title compound (100 mg, 58% yield). ¹H NMR (300 MHz, DMSO-d₆/TFA-d) δ 2.55-2.60 under DMSO (m, 2H), 2.76-2.81 (m, 2H), 7.17-7.23 (m, 3H), 7.28-7.33 (m, 2H), 7.44 (s, 1H), m/z (ES+) M+1 (216); t_{R}=1.13 min.

### Ethyl 4-phenylbutanoate

### Scheme #6, X

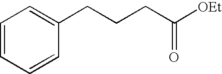

To 4-phenylbutanoic acid (10.00 g, 60.90 mmol) in ethanol (75 mL) was added 2 mL concentrated sulfuric acid. After stirring 2 h the solvent was partially removed under reduced pressure. The remaining material was dissolved in ethyl acetate, washed four times with saturated sodium bicarbonate and once with brine, dried over sodium sulfate, the solvent was removed under reduced pressure, and the resulting material dried under vacuum over night to give the title compound (10.91 g, 93% yield). ¹H NMR (300 MHz, DMSO-d₆/TFA-d) δ 1.18 (t, J=7.3 Hz, 3H), 1.82 (quintet, J=−7.5 Hz, 2H), 2.28 (t, J=7.6 Hz, 2H), 2.58 (t, J=7.7 Hz, 2H), 4.05 (q, J=7.0 Hz, 2H), 7.15-7.20 (m, 3H), 7.26-7.31 (m, 2H), m/z (ES+) M+1 (193); t_{R}=2.33 min.

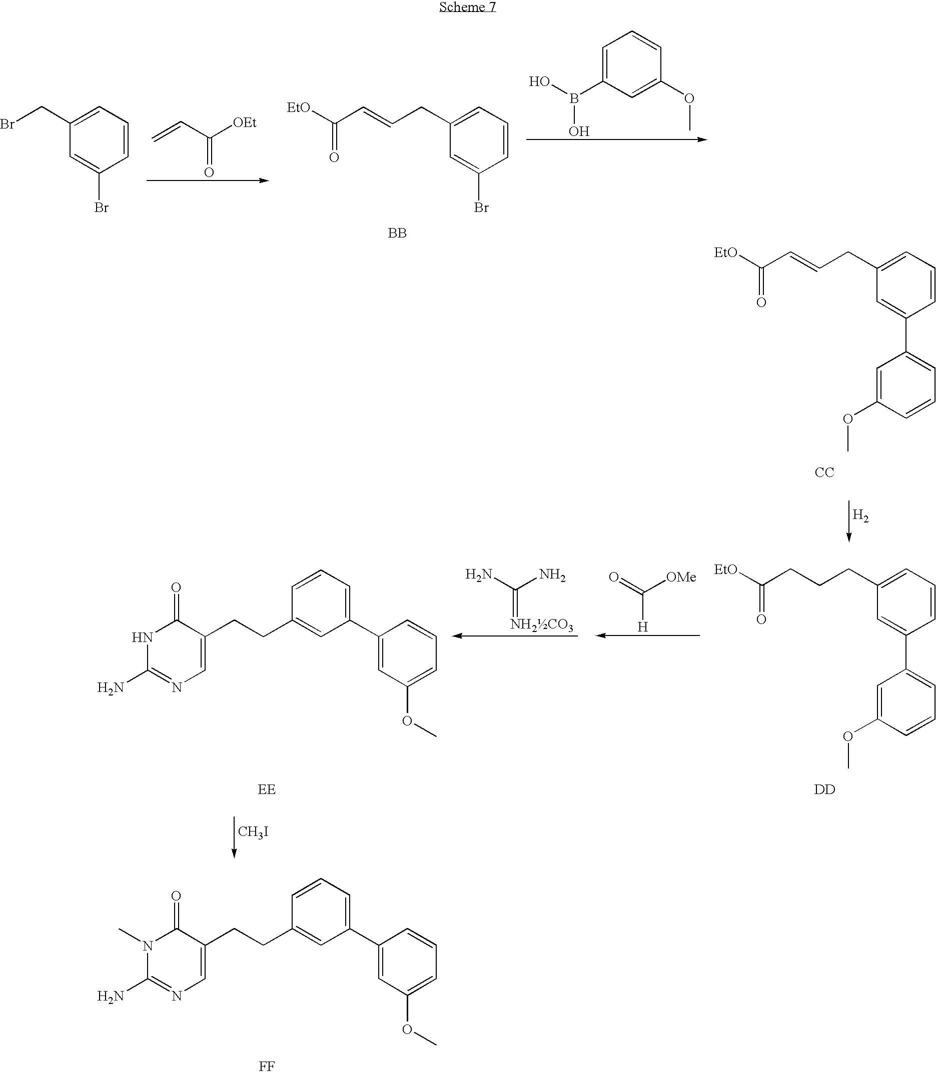

### EXAMPLE 23

### 2-Amino-5-[2-(3′-methoxybiphenyl-3-yl)ethyl]-3-methylpyrimidin-4(3H)-one trifluoroacetate

### Scheme #7, FF

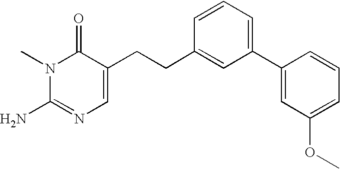

To 2-amino-5-[2-(3′-methoxybiphenyl-3-yl)ethyl]pyrimidin-4(3H)-one trifluoroacetate (170 mg, 0.390 mmol) in ethanol (5.0 mL) was added powdered potassium hydroxide (66 mg, 1.17 mmol). Once homogeneous, methyl iodide (97 uL, 1.56 mmol) was added and the reaction sealed and heated at 80° C. After 3 h the solvent was removed under reduced pressure, the resulting solids dissolved in acetonitrile: water:TFA (75:25:0.1, 4.0 mL) and purified using RP-HPLC AG1 (t_{R}=14.9 min). The combined purified fractions were lyophilized to give the title compound (73 mg, 42% yield). ¹H NMR (300 MHz, DMSO-d₆/TFA-d) δ 2.68 (t, J=7.4 Hz, 2H), 2.87 (t, J=7.6 Hz, 2H), 3.36 (s, 3H), 3.84 (s, 3H), 6.95 (d, J=5.8 Hz, 1H), 7.18-7.24 (m, 3H), 7.38 (t, J=7.7 Hz, 2H), 7.52 (t, J=8.5 Hz, 3H), m/z (APCI+) M+1 (336); t_{R}=2.11 min.

### EXAMPLE 24

### 2-Amino-5-[2-(3′-methoxybiphenyl-3-yl)ethyl]pyrimidin-4(3H)-one trifluoroacetate

### Scheme #7, EE

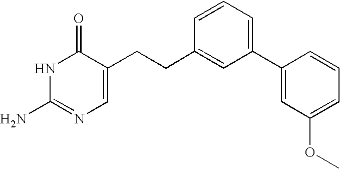

To ethyl 4-(3′-methoxybiphenyl-3-yl)butanoate (258 mg, 0.865 mmol) in THF (4 mL) at −78° C. was added lithium disopropylamide mono(tetrahydrofuran) 1.5 M in cyclohexane (0.63 mL, 0.95 mmol). After 10 min methyl formate (35 uL, 0.57 mmol) was added and reaction stirred for 5 min then warmed to room temperature. The reaction was quenched with ethanol (3.0 mL) and guanidine carbonate (206 mg, 1.14 mmol) was added. The reaction was heated at reflux for 90 min after which the solvents were removed under reduced pressure. The resulting solids were suspended in acetonitrile: water:TFA (75:25:0.1, 4.5 mL), the precipitate filtered off and the filtrate purified using RP-HPLC AG1 (t_{R}=14.5 min). The combined purified fractions were lyophilized to give the title compound (197 mg, 52% yield). ¹H NMR (300 MHz, DMSO-d₆/TFA-d) δ 2.64 (t, J=7.8 Hz, 2H), 2.87 (t, J=7.7 Hz, 2H), 3.84 (s, 3H), 6.95 (d, J=10.5 Hz, 1H), 7.18-7.23 (m, 3H), 7.38 (t, J=7.9 Hz, 2H), 7.50 (d, J=8.6 Hz, 3H), m/z (APCI+) M+1 (322); t_{R}=2.01 min.

### Ethyl 4-(3′-methoxybiphenyl-3-yl)butanoate

### Scheme #7, DD

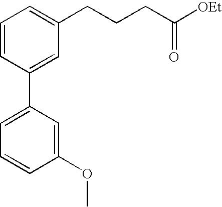

To crude ethyl (2E/Z)-4-(3′-methoxybiphenyl-3-yl)but-2-enoate (1.4 g, 4.7 mmol) was added ethanol (50 mL) and 10% palladium on carbon (300 mg). The reaction was charged with 50 PSI hydrogen and shaken on a Parr Shaker for 1 h. The reaction was filtered through Celite and the solvent removed under reduced pressure to give an orange oil. The oil was purified on a 2×14″ silica column eluting with 7.5% EtOAc/hexanes. The fractions containing pure material were combined and solvent removed under reduced pressure to give the title compound (258 mg, 18% yield). ¹H NMR (300 MHz, DMSO-d₆/TFA-d) δ 1.18 (t, J=7.1 Hz, 3H), 1.90 (quintet, J=7.4 Hz, 2H), 2.32 (t, J=7.4 Hz, 2H), 2.68 (t, J=7.6 Hz, 2H), 3.84 (s, 3H), 4.06 (q, J=7.1 Hz, 2H), 6.92-6.96 (m, 1H), 7.18-7.24 (m, 3H), 7.38 (t, J=8.0 Hz, 2H), 7.47-7.50 (m, 2H), m/z (APCI+) M+1 (299); t_{R}=2.97 min.

### Ethyl (2E/Z)-4-(3′-methoxybiphenyl-3-yl)but-2-enoate

### Scheme #7, CC

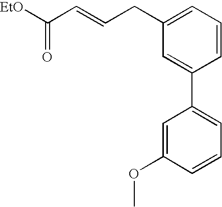

To crude ethyl (2E/Z)-4-(3-bromophenyl)but-2-enoate (2.30 g, 8.55 mmol) was added cesium carbonate (8.35 g, 25.64 mmol), 3-methoxyphenylboronic acid (1.95 g, 12.82 mmol), dichlorobis(triphenylphosphine)palladium(II) (300 mg, 0.427 mmol), and 1,2-dimethoxyethane:water:ethanol (20 mL, 7:3:2). The reaction was heated at reflux in a J-Kem block for 45 min. The aqueous layer was removed and the organic solvents were removed under reduced pressure. The resulting black oil was dissolved in Et₂O, the insoluble material removed by filtration through Celite and the solvent was evaporated under reduced pressure to give an orange oil which was dried under high vacuum. The crude material was chromatographed on silica gel (50 g) eluting with 50% DCM/hexanes to give the title compound (1.4 g, 55% yield) which was used without further purifications in the next reaction.

### Ethyl (2E/Z)-4-(3-bromophenyl)but-2-enoate

### Scheme #7, BB

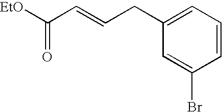

To 1-bromo-3-(bromomethyl)benzene (5.00 g, 20.00 mmol) was added ethyl acrylate (2.4 mL, 22.01 mmol), tri-n-butylamine (3.63 mL, 22.01 mmol), and palladium (II) acetate (0.449 g, 2.00 mmol). The neat reaction was placed in a 110° C. bath for 1 h. To the reaction was added DCM (10 mL) and the mixture was placed on silica gel (50 g) and eluted with DCM to give a crude fractionation of material. The solvent was removed under reduced pressure and the material suspended in 50% DCM/hexanes and applied to a silica gel (50 g) column and eluted with 50% DCM/hexanes. The best looking fractions were combined and the solvents removed under reduced pressure to give the title compound (2.30 g, 43% yield). Used directly in the next reaction.

Various modifications of the invention, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, and the like) cited in the present application is incorporated herein by reference in its entirety.

1. A compound of formula I: 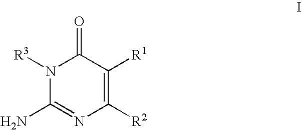
or a pharmaceutically acceptable salt, tautomer or in vivo-hydrolysable precursor thereof, wherein:
R¹ is halo, CN, OR^{a}, SR^{a}, C(O)R^{b}, C(O)NR^{c}R^{d}, C(O)OR^{a}, OC(O)R^{b}, OC(O)NR^{c}R^{d}, S(O)R^{b}, S(O)NR^{c}R^{d}, S(O)₂R^{b}, S(O)₂NR^{c}R^{d}, C₁₋₆ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl, wherein each of the C₁₋₆ alkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl is optionally substituted by 1, 2, 3, 4 or 5 A¹;
R² is —(CR^{2a}R^{2b})₂-Q;
R³ is H, C(O)R^{a}, C(O)OR^{b}, C(O)NR^{c}R^{d}, S(O)R^{a}, S(O)₂R^{a}, C₁₋₁₀ alkyl, C₂₋₁₀ alkenyl, C₂₋₁₀ alkynyl, cycloalkyl, heterocycloalkyl, arylalkyl, heteroarylalkyl, cycloalkylalkyl or heterocycloalkylalkyl, wherein the C₁₋₁₀ alkyl, C₂₋₁₀ alkenyl, C₂₋₁₀ alkynyl, cycloalkyl, heterocycloalkyl, arylalkyl, heteroarylalkyl, cycloalkylalkyl or heterocycloalkylalkyl are each optionally substituted with 1, 2, 3, 4 or 5 A²;
R^{2a} and R^{2b} are each, independently, H, halo, C₁₋₄ alkyl, C₁₋₄ haloalkyl, C₂₋₆ alkenyl, C₂₋₆ alkynyl, aryl, cycloalkyl, heteroaryl, heterocycloalkyl, CN, NO₂, OR^{a′}, SR^{a′}, C(O)R^{b′}, C(O)NR^{c′}R^{d′}, C(O)OR^{a′}, OC(O)R^{b′}, OC(O)NR^{c′}R^{d′}, NR^{c′}R^{d′}, NR^{c′}C(O)R^{d′}, NR^{c′}C(O)OR^{a′}, NR^{c′}S(O)₂R^{b′}, S(O)R^{b′}, S(O)NR^{c′}R^{d′}, S(O)₂R^{b′}, or S(O)₂NR^{c′}R^{d′};
Q is aryl, cycloalkyl, heteroaryl or heterocycloalkyl, each optionally substituted by 1, 2, 3, 4 or 5 Cy¹ or R^{Q};
Cy¹ is aryl, heteroaryl, cycloalkyl, or heterocycloalkyl, each optionally substituted with 1, 2, 3, 4 or 5 A³;
A¹, A², and A³ are each, independently, halo, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, NR^{c}S(O)R^{b}, NR^{c}S(O)₂R^{b}, S(O)R^{b}, S(O)NR^{c}R^{d}, S(O)₂R^{b}, S(O)₂NR^{c}R^{d}, C₁₋₄ alkoxy, C₁₋₄ haloalkoxy, amino, C₁₋₄ alkylamino, C₂₋₈ dialkylamino, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl or heterocycloalkyl, wherein each of the C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl or heterocycloalkyl is optionally substituted by 1, 2, 3, 4 or 5 halo, C₁₋₆ alkyl, C₂₋₆ alkenyl, C₂₋₆ alkynyl, C₁₋₄ haloalkyl, aryl, cycloalkyl, heteroaryl, heterocycloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, NR^{c}S(O)R^{b}, NR^{c}S(O)₂R^{b}, S(O)R^{b}, S(O)NR^{c}R^{d}, S(O)₂R^{b}, or S(O)₂NR^{c}R^{d};
R^{Q} is halo, CN, NO₂, OR^{a}, SR^{a}, C(O)R^{b}, C(O)NR^{c}R^{d}, C(O)OR^{a}, OC(O)R^{b}, OC(O)NR^{c}R^{d}, S(O)R^{b}, S(O)NR^{c}R^{d}, S(O)₂R^{b}, S(O)₂NR^{c}R^{d}, C₁₋₄ alkoxy, C₁₋₄ haloalkoxy, C₁₋₆ alkyl, C₂₋₆ alkenyl, C₂₋₆ alkynyl, arylalkyl, cycloalkylalkyl, heteroarylalkyl or heterocycloalkylalkyl, wherein each of the C₁₋₆ alkyl, C₂₋₆ alkenyl, C₂₋₆ alkynyl, arylalkyl, cycloalkylalkyl, heteroarylalkyl or heterocycloalkylalkyl is optionally substituted by 1, 2, 3, 4 or 5 halo, C₁₋₆ alkyl, C₂₋₆ alkenyl, C₂₋₆ alkynyl, C₁₋₄ haloalkyl, aryl, cycloalkyl, heteroaryl, heterocycloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, NR^{c}S(O)R^{b}, NR^{c}S(O)₂R^{b}, S(O)R^{b}, S(O)NR^{c}R^{d}, S(O)₂R^{b}, or S(O)₂NR^{c}R^{d};
R^{a} and R^{a′} are each, independently, H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl, wherein the C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl is optionally substituted with OH, amino, halo, C₁₋₆ alkyl, C₁₋₆ haloalkyl, aryl, arylalkyl, heteroaryl, heteroarylalkyl, cycloalkyl or heterocycloalkyl;
R^{b} and R^{b′} are each, independently, H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl, wherein the C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl is optionally substituted with OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;
R^{c} and R^{d} are each, independently, H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl, wherein the C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl is optionally substituted with OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;
or R^{c} and R^{d} together with the N atom to which they are attached form a 4-, 5-, 6- or 7-membered heterocycloalkyl group; and
R^{c′} and R^{d′} are each, independently, H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl, wherein the C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl is optionally substituted with OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;
or R^{c′} and R^{d′} together with the N atom to which they are attached form a 4-, 5-, 6- or 7-membered heterocycloalkyl group.

2. A compound of claim 1 wherein R¹ is halo, C₁₋₆ alkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl, wherein each of the C₁₋₆ alkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl is optionally substituted by 1, 2 or 3 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl.

3. A compound of claim 1 wherein R¹ is halo, C₁₋₆ alkyl, aryl, heteroaryl, cycloalkyl, heterocycloalkyl, arylalkyl, heteroarylalkyl, cycloalkylalkyl or heterocycloalkylalkyl.

4. A compound of claim 1 wherein R^{2a} and R^{2b} are each, independently, H, halo, C₁₋₄ alkyl, C₁₋₄ haloalkyl, C₂₋₆ alkenyl, C₂₋₆ alkynyl, aryl, cycloalkyl, heteroaryl, heterocycloalkyl, NO₂, OR^{a′}, SR^{a′}, OC(O)R^{b′}, OC(O)NR^{c′}R^{d′}, S(O)R^{b′}, S(O)NR^{c′}R^{d′}, S(O)₂R^{b′}, or S(O)₂NR^{c′}R^{d′}.

5. A compound of claim 1 wherein R^{2a} and R^{2b} are each, independently, H, halo, C₁₋₄ alkyl, C₁₋₄ haloalkyl, C₂₋₆ alkenyl, C₂₋₆ alkynyl, aryl, cycloalkyl, heteroaryl or heterocycloalkyl.

6. A compound of claim 1 wherein R^{2a} and R^{2b} are each, independently, H or C₁₋₄ alkyl.

7. A compound of claim 1 wherein R^{2a} and R^{2b} are both H.

8. A compound of claim 1 wherein Q is aryl or heteroaryl, each optionally substituted by 1, 2, 3, 4 or 5 Cy¹ or R^{Q}.

9. A compound of claim 1 wherein Q is aryl optionally substituted by 1, 2, 3, 4 or 5 Cy¹ or R^{Q}.

10. A compound of claim 1 wherein Q is aryl optionally substituted by 1, 2 or 3 R^{Q}.

11. A compound of claim 1 wherein Q is aryl substituted by Cy¹ and optionally substituted by 1, 2 or 3 R^{Q}.

12. A compound of claim 1 wherein:
Q is aryl substituted by Cy¹ and optionally substituted by 1, 2 or 3 R^{Q}; and
Cy¹ is aryl or heteroaryl, each optionally substituted by 1, 2, 3, 4 or 5 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl.

13. A compound of claim 1 wherein:
Q is phenyl wherein the phenyl is meta-substituted by Cy¹; and
Cy¹ is aryl or heteroaryl, each optionally substituted by 1, 2, 3, 4 or 5 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl.

14. A compound of claim 1 wherein:
Q is phenyl wherein the phenyl is meta-substituted by Cy¹; and
Cy¹ is aryl optionally substituted by 1, 2, 3, 4 or 5 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl.

15. A compound of claim 1 wherein R³ is C₁₋₁₀ alkyl, C₂₋₁₀ alkenyl, C₂₋₁₀ alkynyl, cycloalkyl, heterocycloalkyl, arylalkyl, heteroarylalkyl, cycloalkylalkyl or heterocycloalkylalkyl, wherein the C₁₋₁₀ alkyl, C₂₋₁₀ alkenyl, C₂₋₁₀ alkynyl, cycloalkyl, heterocycloalkyl, arylalkyl, heteroarylalkyl, cycloalkylalkyl or heterocycloalkylalkyl are each optionally substituted with 1, 2, 3, 4 or 5 A².

16. A compound of claim 1 wherein R³ is C₁₋₁₀ alkyl, arylalkyl, heteroarylalkyl, cycloalkylalkyl or heterocycloalkylalkyl, wherein the C₁₋₁₀ alkyl, arylalkyl, heteroarylalkyl, cycloalkylalkyl or heterocycloalkylalkyl are each optionally substituted with 1, 2, 3, 4 or 5 A².

17. A compound of claim 1 wherein:
R³ is C₁₋₁₀ alkyl, arylalkyl, heteroarylalkyl, cycloalkylalkyl or heterocycloalkylalkyl, wherein the C₁₋₁₀ alkyl, arylalkyl, heteroarylalkyl, cycloalkylalkyl or heterocycloalkylalkyl are each optionally substituted with 1, 2 or 3 A²; and
A² is halo, CN, NO₂, OR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, NR^{c}S(O)₂R^{b}, S(O)₂R^{b}, S(O)₂NR^{c}R^{d}, C₁₋₄ alkoxy, C₁₋₄ haloalkoxy, amino, C₁₋₄ alkylamino, C₂₋₈ dialkylamino, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl or heterocycloalkyl, wherein each of the C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl or heterocycloalkyl is optionally substituted by 1, 2, 3, 4 or 5 halo, C₁₋₆ alkyl, C₂₋₆ alkenyl, C₂₋₆ alkynyl, C₁₋₄ haloalkyl, aryl, cycloalkyl, heteroaryl, heterocycloalkyl, CN, NO₂, OR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, NR^{c}S(O)₂R^{b}, S(O)R^{b}, S(O)₂R^{b}, or S(O)₂NR^{c}R^{d}.

18. A compound of claim 1 wherein R³ is C₁₋₁₀ alkyl, arylalkyl, heteroarylalkyl, cycloalkylalkyl or heterocycloalkylalkyl.

19. A compound of claim 1 wherein R³ is C₁₋₁₀ alkyl.

20. A compound of claim 1 wherein:
R¹ is halo, C₁₋₆ alkyl, aryl, heteroaryl, cycloalkyl, heterocycloalkyl, arylalkyl, heteroarylalkyl, cycloalkylalkyl or heterocycloalkylalkyl;
R^{2a} and R^{2b} are each, independently, H or C₁₋₄ alkyl;
Q is aryl optionally substituted by 1, 2 or 3 R^{Q}; and
R³ is C₁₋₁₀ alkyl, arylalkyl, heteroarylalkyl, cycloalkylalkyl or heterocycloalkylalkyl, wherein the C₁₋₁₀ alkyl, arylalkyl, heteroarylalkyl, cycloalkylalkyl or heterocycloalkylalkyl are each optionally substituted with 1, 2, 3, 4 or 5 A².

21. A compound of claim 1 wherein:
R¹ is halo, C₁₋₆ alkyl, aryl, heteroaryl, cycloalkyl, heterocycloalkyl, arylalkyl, heteroarylalkyl, cycloalkylalkyl or heterocycloalkylalkyl;
R^{2a} and R^{2b} are each, independently, H or C₁₋₄ alkyl;
Q is phenyl optionally substituted by 1, 2 or 3 halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, or heterocycloalkylalkyl; and
R³ is C₁₋₁₀ alkyl, arylalkyl, heteroarylalkyl, cycloalkylalkyl or heterocycloalkylalkyl, wherein the C₁₋₁₀ alkyl, arylalkyl, heteroarylalkyl, cycloalkylalkyl or heterocycloalkylalkyl are each optionally substituted with 1, 2, 3, 4 or 5 halo, CN, NO₂, OR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, NR^{c}S(O)₂R^{b}, S(O)₂R^{b}, S(O)₂NR^{c}R^{d}, C₁₋₄ alkoxy, C₁₋₄ haloalkoxy, amino, C₁₋₄ alkylamino, C₂₋₈ dialkylamino, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl or heterocycloalkyl.

22. A compound of claim 21 wherein Q is phenyl meta-substituted by halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, or heterocycloalkylalkyl.

23. A compound of claim 1, wherein the compound has the structure of formula II: 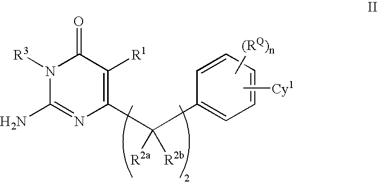
wherein:
R¹ is halo, C₁₋₆ alkyl, aryl, heteroaryl, cycloalkyl, heterocycloalkyl, arylalkyl, heteroarylalkyl, cycloalkylalkyl or heterocycloalkylalkyl;
R^{2a} and R^{2b} are each, independently, H or C₁₋₄ alkyl;
R³ is C₁₋₁₀ alkyl, arylalkyl, heteroarylalkyl, cycloalkylalkyl or heterocycloalkylalkyl, wherein the C₁₋₁₀ alkyl, arylalkyl, heteroarylalkyl, cycloalkylalkyl or heterocycloalkylalkyl are each optionally substituted with 1, 2, 3, 4 or 5 halo, CN, NO₂, OR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, NR^{c}S(O)₂R^{b}, S(O)₂R^{b}, S(O)₂NR^{c}R^{d}, C₁₋₄ alkoxy, C₁₋₄ haloalkoxy, amino, C₁₋₄ alkylamino, C₂₋₈ dialkylamino, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl or heterocycloalkyl;
R^{Q} is halo, CN, C₁₋₄ alkoxy, C₁₋₄ haloalkoxy, C₁₋₆ alkyl, C₂₋₆ alkenyl, C₂₋₆ alkynyl, arylalkyl, cycloalkylalkyl, heteroarylalkyl or heterocycloalkylalkyl;
Cy¹ is aryl or heteroaryl, each optionally substituted by 1, 2, 3, 4 or 5 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl; and
n is 0 or 1.

24. A compound of claim 1, wherein the compound has the structure of formula III: 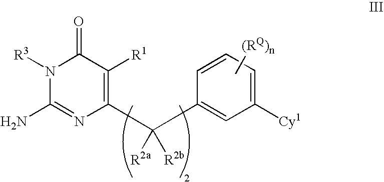
wherein:
R¹ is halo, C₁₋₆ alkyl, aryl, heteroaryl, cycloalkyl, heterocycloalkyl, arylalkyl, heteroarylalkyl, cycloalkylalkyl or heterocycloalkylalkyl;
R^{2a} and R^{2b} are each, independently, H or C₁₋₄ alkyl;
R³ is C₁₋₁₀ alkyl, arylalkyl, heteroarylalkyl, cycloalkylalkyl or heterocycloalkylalkyl, wherein the C₁₋₁₀ alkyl, arylalkyl, heteroarylalkyl, cycloalkylalkyl or heterocycloalkylalkyl are each optionally substituted with 1, 2, 3, 4 or 5 halo, CN, NO₂, OR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, NR^{c}S(O)₂R^{b}, S(O)₂R^{b}, S(O)₂NR^{c}R^{d}, C₁₋₄ alkoxy, C₁₋₄ haloalkoxy, amino, C₁₋₄ alkylamino, C₂₋₈ dialkylamino, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl or heterocycloalkyl;
R^{Q} is halo, CN, C₁₋₄ alkoxy, C₁₋₄ haloalkoxy, C₁₋₆ alkyl, C₂₋₆ alkenyl, C₂₋₆ alkynyl, arylalkyl, cycloalkylalkyl, heteroarylalkyl or heterocycloalkylalkyl;
Cy¹ is aryl or heteroaryl, each optionally substituted by 1, 2, 3, 4 or 5 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl; and
n is 0 or 1.

25. A compound of claim 24 wherein n is 0.

26. A compound of claim 24 wherein:
n is 0;
Cy¹ is phenyl optionally substituted by 1, 2, 3, 4 or 5 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl.

27. A compound of formula IV: 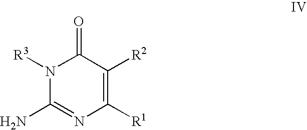
or a pharmaceutically acceptable salt, tautomer or in vivo-hydrolysable precursor thereof, wherein:
R¹ is H, halo, CN, OR^{a}, SR^{a}, C(O)R^{b}, C(O)NR^{c}R^{d}, C(O)OR^{a}, OC(O)R^{b}, OC(O)NR^{c}R^{d}, S(O)R^{b}, S(O)NR^{c}R^{d}, S(O)₂R^{b}, S(O)₂NR^{c}R^{d}, C₁₋₆ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl, wherein each of the C₁₋₆ alkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl is optionally substituted by 1, 2, 3, 4 or 5 A¹;
R² is —(CR^{2a}R^{2b})₂—Q;
R³ is C(O)R^{a}, C(O)OR^{b}, C(O)NR^{c}R^{d}, S(O)R^{a}, S(O)₂R^{a}, C₁₋₁₀ alkyl, C₂₋₁₀ alkenyl, C₂₋₁₀ alkynyl, cycloalkyl, heterocycloalkyl, arylalkyl, heteroarylalkyl, cycloalkylalkyl or heterocycloalkylalkyl, wherein the C₁₋₁₀ alkyl, C₂₋₁₀ alkenyl, C₂₋₁₀ alkynyl, cycloalkyl, heterocycloalkyl, arylalkyl, heteroarylalkyl, cycloalkylalkyl or heterocycloalkylalkyl are each optionally substituted with 1, 2, 3, 4 or 5 A²;
R^{2a} and R^{2b} are each, independently, H, halo, C₁₋₄ alkyl, C₁₋₄ haloalkyl, C₂₋₆ alkenyl, C₂₋₆ alkynyl, aryl, cycloalkyl, heteroaryl, heterocycloalkyl, CN, NO₂, OR^{a′}, SR^{a′}, C(O)R^{b′}, C(O)NR^{c′}R^{d′}, C(O)OR^{a′}, OC(O)R^{b′}, OC(O)NR^{c′}R^{d′}, NR^{c′}R^{d′}, NR^{c′}C(O)R^{d′}, NR^{c′}C(O)OR^{a′}, NR^{c′}S(O)₂R^{b′}, S(O)R^{b′}, S(O)NR^{c′}R^{d′}, S(O)₂R^{b′}, or S(O)₂NR^{c′}R^{d′};
Q is aryl, heteroaryl or cycloalkyl, each optionally substituted by 1, 2, 3, 4 or 5 Cy¹ or R^{Q};
Cy¹ is aryl, heteroaryl or cycloalkyl, each optionally substituted with 1, 2, 3, 4 or 5 A³;
A¹, A², and A³ are each, independently, halo, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, NR^{c}S(O)R^{b}, NR^{c}S(O)₂R^{b}, S(O)R^{b}, S(O)NR^{c}R^{d}, S(O)₂R^{b}, S(O)₂NR^{c}R^{d}, C₁₋₄ alkoxy, C₁₋₄ haloalkoxy, amino, C₁₋₄ alkylamino, C₂₋₈ dialkylamino, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl or heterocycloalkyl, wherein each of the C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl or heterocycloalkyl is optionally substituted by 1, 2, 3, 4 or 5 halo, C₁₋₆ alkyl, C₂₋₆ alkenyl, C₂₋₆ alkynyl, C₁₋₄ haloalkyl, aryl, cycloalkyl, heteroaryl, heterocycloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, NR^{c}S(O)R^{b}, NR^{c}S(O)₂R^{b}, S(O)R^{b}, S(O)NR^{c}R^{d}, S(O)₂R^{b}, or S(O)₂NR^{c}R^{d};
R^{Q} is halo, CN, NO₂, OR^{a}, SR^{a}, C(O)R^{b}, C(O)NR^{c}R^{d}, C(O)OR^{a}, OC(O)R^{b}, OC(O)NR^{c}R^{d}, S(O)R^{b}, S(O)NR^{c}R^{d}, S(O)₂R^{b}, S(O)₂NR^{c}R^{d}, C₁₋₄ alkoxy, C₁₋₄ haloalkoxy, C₁₋₆ alkyl, C₂₋₆ alkenyl, C₂₋₆ alkynyl, arylalkyl, cycloalkylalkyl, heteroarylalkyl or heterocycloalkylalkyl, wherein each of the C₁₋₆ alkyl, C₂₋₆ alkenyl, C₂₋₆ alkynyl, arylalkyl, cycloalkylalkyl, heteroarylalkyl or heterocycloalkylalkyl is optionally substituted by 1, 2, 3, 4 or 5 halo, C₁₋₆ alkyl, C₂₋₆ alkenyl, C₂₋₆ alkynyl, C₁₋₄ haloalkyl, aryl, cycloalkyl, heteroaryl, heterocycloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, NR^{c}S(O)R^{b}, NR^{c}S(O)₂R^{b}, S(O)R^{b}, S(O)NR^{c}R^{d}, S(O)₂R^{b}, or S(O)₂NR^{c}R^{d};
R^{a} and R^{a′} are each, independently, H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl, wherein the C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl is optionally substituted with OH, amino, halo, C₁₋₆ alkyl, C₁₋₆ haloalkyl, aryl, arylalkyl, heteroaryl, heteroarylalkyl, cycloalkyl or heterocycloalkyl;
R^{b} and R^{b′} are each, independently, H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl, wherein the C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl is optionally substituted with OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;
R^{c} and R^{d} are each, independently, H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl, wherein the C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl is optionally substituted with OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;
or R^{c} and R^{d} together with the N atom to which they are attached form a 4-, 5-, 6- or 7-membered heterocycloalkyl group; and
R^{c′} and R^{d′} are each, independently, H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl, wherein the C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl is optionally substituted with OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;
or R^{c′} and R^{d′} together with the N atom to which they are attached form a 4-, 5-, 6- or 7-membered heterocycloalkyl group.

28-48. (canceled)

49. A compound of formula VII: 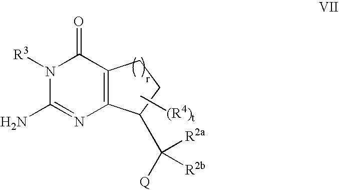
or a pharmaceutically acceptable salt, tautomer or in vivo-hydrolysable precursor thereof, wherein:
R³ is H, C(O)R^{a}, C(O)OR^{b}, C(O)NR^{c}R^{d}, S(O)R^{a}, S(O)₂R^{a}, C₁₋₁₀ alkyl, C₂₋₁₀ alkenyl, C₂₋₁₀ alkynyl, cycloalkyl, heterocycloalkyl, arylalkyl, heteroarylalkyl, cycloalkylalkyl or heterocycloalkylalkyl, wherein the C₁₋₁₀ alkyl, C₂₋₁₀ alkenyl, C₂₋₁₀ alkynyl, cycloalkyl, heterocycloalkyl, arylalkyl, heteroarylalkyl, cycloalkylalkyl or heterocycloalkylalkyl are each optionally substituted with 1, 2, 3, 4 or 5 A²;
R⁴ is halo, CN, OR^{a}, SR^{a}, C(O)R^{b}, C(O)NR^{c}R^{d}, C(O)OR^{a}, OC(O)R^{b}, OC(O)NR^{c}R^{d}, S(O)R^{b}, S(O)NR^{c}R^{d}, S(O)₂R^{b}, S(O)₂NR^{c}R^{d}, C₁₋₆ alkyl, C₁₋₆ haloalkyl, aryl, heteroaryl, cycloalkyl, heterocycloalkyl, arylalkyl, heteroarylalkyl, cycloalkylalkyl or heterocycloalkylalkyl, wherein the C₁₋₆ alkyl, aryl, heteroaryl, cycloalkyl, heterocycloalkyl, arylalkyl, heteroarylalkyl, cycloalkylalkyl or heterocycloalkylalkyl is optionally substituted by 1, 2, 3, 4 or 5 A¹;
R^{2a} and R^{2b} are each, independently, H, halo, C₁₋₄ alkyl, C₁₋₄ haloalkyl, C₂₋₆ alkenyl, C₂₋₆ alkynyl, aryl, cycloalkyl, heteroaryl, heterocycloalkyl, CN, NO₂, OR^{a′}, SR^{a′}, C(O)R^{b′}, C(O)NR^{c′}R^{d′}, C(O)OR^{a′}, OC(O)R^{b′}, OC(O)NR^{c′}R^{d′}, NR^{c′}R^{d′}, NR^{c′}C(O)R^{d′}, NR^{c′}C(O)OR^{a′}, NR^{c′}S(O)₂R^{b′}, S(O)R^{b′}, S(O)NR^{c′}R^{d′}, S(O)₂R^{b′}, or S(O)₂NR^{c′}R^{d′};
r is 0, 1, 2 or 3;
t is 0, 1, 2, 3, 4 or 5;
Q is aryl, cycloalkyl, heteroaryl or heterocycloalkyl, each optionally substituted by 1, 2, 3, 4 or 5 Cy¹ or R^{Q};
Cy¹ is aryl, heteroaryl, cycloalkyl, or heterocycloalkyl, each optionally substituted with 1, 2, 3, 4 or 5 A³;
A¹, A ², and A³ are each, independently, halo, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, NR^{c}S(O)R^{b}, NR^{c}S(O)₂R^{b}, S(O)R^{b}, S(O)NR^{c}R^{d}, S(O)₂R^{b}, S(O)₂NR^{c}R^{d}, C₁₋₄ alkoxy, C₁₋₄ haloalkoxy, amino, C₁₋₄ alkylamino, C₂₋₈ dialkylamino, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl or heterocycloalkyl, wherein each of the C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl or heterocycloalkyl is optionally substituted by 1, 2, 3, 4 or 5 halo, C₁₋₆ alkyl, C₂₋₆ alkenyl, C₂₋₆ alkynyl, C₁₋₄ haloalkyl, aryl, cycloalkyl, heteroaryl, heterocycloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, NR^{c}S(O)R^{b}, NR^{c}S(O)₂R^{b}, S(O)R^{b}, S(O)NR^{c}R^{d}, S(O)₂R^{b}, or S(O)₂NR^{c}R^{d};
R^{Q} is halo, CN, NO₂, OR^{a}, SR^{a}, C(O)R^{b}, C(O)NR^{c}R^{d}, C(O)OR^{a}, OC(O)R^{b}, OC(O)NR^{c}R^{d}, S(O)R^{b}, S(O)NR^{c}R^{d}, S(O)₂R^{b}, S(O)₂NR^{c}R^{d}, C₁₋₄ alkoxy, C₁₋₄ haloalkoxy, C₁₋₆ alkyl, C₂₋₆ alkenyl, C₂₋₆ alkynyl, arylalkyl, cycloalkylalkyl, heteroarylalkyl or heterocycloalkylalkyl, wherein each of the C₁₋₆ alkyl, C₂₋₆ alkenyl, C₂₋₆ alkynyl, arylalkyl, cycloalkylalkyl, heteroarylalkyl or heterocycloalkylalkyl is optionally substituted by 1, 2, 3, 4 or 5 halo, C₁₋₆ alkyl, C₂₋₆ alkenyl, C₂₋₆ alkynyl, C₁₋₄ haloalkyl, aryl, cycloalkyl, heteroaryl, heterocycloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, NR^{c}S(O)R^{b}, NR^{c}S(O)₂R^{b}, S(O)R^{b}, S(O)NR^{c}R^{d}, S(O)₂R^{b}, or S(O)₂NR^{c}R^{d};
R^{a} and R^{a′} are each, independently, H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl, wherein the C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl is optionally substituted with OH, amino, halo, C₁₋₆ alkyl, C₁₋₆ haloalkyl, aryl, arylalkyl, heteroaryl, heteroarylalkyl, cycloalkyl or heterocycloalkyl;
R^{b} and R^{b′} are each, independently, H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl, wherein the C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl is optionally substituted with OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;
R^{c} and R^{d} are each, independently, H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl, wherein the C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl is optionally substituted with OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;
or R^{c} and R^{d} together with the N atom to which they are attached form a 4-, 5-, 6- or 7-membered heterocycloalkyl group; and
R^{c′} and R^{d′} are each, independently, H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl, wherein the C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl is optionally substituted with OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;
or R^{c′} and R^{d′} together with the N atom to which they are attached form a 4-, 5-, 6- or 7-membered heterocycloalkyl group.

50-73. (canceled)

74. A compound of formula XI: 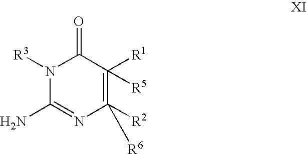
or a pharmaceutically acceptable salt, tautomer or in vivo-hydrolysable precursor thereof, wherein:
R¹ is C₁₋₆ alkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl, each optionally substituted by 1, 2, 3, 4 or 5 A¹;
R² is —(CR^{2a}R^{2b})ₘ-Q;
R³ is H, C(O)R^{a}, C(O)OR^{b}, C(O)NR^{c}R^{d}, S(O)R^{a}, S(O)₂R^{a}, C₁₋₁₀ alkyl, C₂₋₁₀ alkenyl, C₂₋₁₀ alkynyl, cycloalkyl, heterocycloalkyl, arylalkyl, heteroarylalkyl, cycloalkylalkyl or heterocycloalkylalkyl, wherein the C₁₋₁₀ alkyl, C₂₋₁₀ alkenyl, C₂₋₁₀ alkynyl, cycloalkyl, heterocycloalkyl, arylalkyl, heteroarylalkyl, cycloalkylalkyl or heterocycloalkylalkyl are each optionally substituted with 1, 2, 3, 4 or 5 A²;
R⁵ is H, C₁₋₆ alkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl, wherein each of the C₁₋₆ alkyl, aryl, heteroaryl, cycloalkyl, heterocycloalkyl, arylalkyl, heteroarylalkyl, cycloalkylalkyl or heterocycloalkylalkyl is optionally substituted by 1, 2, 3, 4 or 5 A⁴,
R⁶ is H, C₁₋₆ alkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl, wherein each of the C₁₋₆ alkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl is optionally substituted by 1, 2, 3, 4 or 5 A⁵;
R^{2a} and R^{2b} are each, independently, H, halo, C₁₋₄ alkyl, C₁₋₄ haloalkyl, C₂₋₆ alkenyl, C₂₋₆ alkynyl, aryl, cycloalkyl, heteroaryl, heterocycloalkyl, CN, NO₂, OR^{a′}, SR^{a′}, C(O)R^{b′}, C(O)NR^{c′}R^{d′}, C(O)OR^{a′}, OC(O)R^{b′}, OC(O)NR^{c′}R^{d′}, NR^{c′}R^{d′}, NR^{c′}C(O)R^{d′}, NR^{c′}C(O)OR^{a′}, NR^{c′}S(O)₂R^{b′}, S(O)R^{b′}, S(O)NR^{c′}R^{d′}, S(O)₂R^{b′}, or S(O)₂NR^{c′}R^{d′};
m is 0, 1, 2, 3 or 4;
Q is aryl, cycloalkyl, heteroaryl or heterocycloalkyl, each optionally substituted by 1, 2, 3, 4 or 5 Cy¹ or R^{Q};
Cy¹ is aryl, heteroaryl, cycloalkyl, or heterocycloalkyl, each optionally substituted with 1, 2, 3, 4 or 5 A³;
A¹, A², A³, A⁴, and A⁵ are each, independently, halo, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, NR^{c}S(O)R^{b}, NR^{c}S(O)₂R^{b}, S(O)R^{b}, S(O)NR^{c}R^{d}, S(O)₂R^{b}, S(O)₂NR^{c}R^{d}, C₁₋₄ alkoxy, C₁₋₄ haloalkoxy, amino, C₁₋₄ alkylamino, C₂₋₈ dialkylamino, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl or heterocycloalkyl, wherein each of the C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl or heterocycloalkyl is optionally substituted by 1, 2, 3, 4 or 5 halo, C₁₋₆ alkyl, C₂₋₆ alkenyl, C₂₋₆ alkynyl, C₁₋₄ haloalkyl, aryl, cycloalkyl, heteroaryl, heterocycloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, NR^{c}S(O)R^{b}, NR^{c}S(O)₂R^{b}, S(O)R^{b}, S(O)NR^{c}R^{d}, S(O)₂R^{b}, or S(O)₂NR^{c}R^{d};
R^{Q} is halo, CN, NO₂, OR^{a}, SR^{a}, C(O)R^{b}, C(O)NR^{c}R^{d}, C(O)OR^{a}, OC(O)R^{b}, OC(O)NR^{c}R^{d}, S(O)R^{b}, S(O)NR^{c}R^{d}, S(O)₂R^{b}, S(O)₂NR^{c}R^{d}, C₁₋₄ alkoxy, C₁₋₄ haloalkoxy, C₁₋₆ alkyl, C₂₋₆ alkenyl, C₂₋₆ alkynyl, arylalkyl, cycloalkylalkyl, heteroarylalkyl or heterocycloalkylalkyl, wherein each of the C₁₋₆ alkyl, C₂₋₆ alkenyl, C₂₋₆ alkynyl, arylalkyl, cycloalkylalkyl, heteroarylalkyl or heterocycloalkylalkyl is optionally substituted by 1, 2, 3, 4 or 5 halo, C₁₋₆ alkyl, C₂₋₆ alkenyl, C₂₋₆ alkynyl, C₁₋₄ haloalkyl, aryl, cycloalkyl, heteroaryl, heterocycloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, NR^{c}S(O)R^{b}, NR^{c}S(O)₂R^{b}, S(O)R^{b}, S(O)NR^{c}R^{d}, S(O)₂R^{b}, or S(O)₂NR^{c}R^{d};
R^{a} and R^{a′} are each, independently, H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl, wherein the C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl is optionally substituted with OH, amino, halo, C₁₋₆ alkyl, C₁₋₆ haloalkyl, aryl, arylalkyl, heteroaryl, heteroarylalkyl, cycloalkyl or heterocycloalkyl;
R^{b} and R^{b′} are each, independently, H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl, wherein the C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl is optionally substituted with OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;
R^{c} and R^{d} are each, independently, H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl, wherein the C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl is optionally substituted with OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;
or R^{c} and R^{d} together with the N atom to which they are attached form a 4-, 5-, 6- or 7-membered heterocycloalkyl group; and
R^{c′} and R^{d′} are each, independently, H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl, wherein the C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl is optionally substituted with OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;
or R^{c′} and R^{d′} together with the N atom to which they are attached form a 4-, 5-, 6- or 7-membered heterocycloalkyl group.

75-100. (canceled)

101. A compound selected from:
2-amino-6-(3-bromo-4-chlorophenyl)-5,6-dimethyl-5,6-dihydropyrimidin-4(3H)-one trifluoroacetate;
2-amino-6-[2-(3′-methoxybiphenyl-3-yl)ethyl]-3,5-dimethylpyrimidin-4(3H)-one trifluoroacetate;
2-amino-3,5-dimethyl-6-(2-phenylethyl)pyrimidin-4(3H)-one trifluoroacetate;
2-amino-6-{2-[3-(2-furyl)phenyl]ethyl}-3,5-dimethylpyrimidin-4(3H)-one trifluoroacetate;
2-amino-6-[2-(3-bromophenyl)ethyl]-3,5-dimethylpyrimidin-4(3H)-one;
2-amino-6-[2-(3-bromophenyl)ethyl]-5-methylpyrimidin-4(3H)-one;
2-amino-5-benzyl-6-[2-(3′-methoxybiphenyl-3-yl)ethyl]-3-methylpyrimidin-4(3H)-one trifluoroacetate;
2-amino-5-benzyl-6-[2-(3-bromophenyl)ethyl]-3-methylpyrimidin-4(3H)-one trifluoroacetate;
2-amino-3-methyl-5-phenyl-6-(2-phenylethyl)pyrimidin-4(3H)-one trifluoroacetate;
2-amino-5-bromo-3-methyl-6-(2-phenylethyl)pyrimidin-4(3H)-one;
2-amino-3-methyl-6-(2-phenylethyl)pyrimidin-4(3H)-one;
2-amino-6-(2-phenylethyl)pyrimidin-4(3H)-one;
2-amino-6-[2-(3-bromophenyl)ethyl]pyrimidin-4(3H)-one;
2-amino-8-[(3′-methoxybiphenyl-3-yl)methyl]-3-methyl-5,6,7,8-tetrahydroquinazolin-4(3H)-one trifluoroacetate;
2-amino-8-(3-bromobenzyl)-3-methyl-5,6,7,8-tetrahydroquinazolin-4(3H)-one;
2-amino-8-(3-bromobenzyl)-5,6,7,8-tetrahydroquinazolin-4(3H)-one trifluoroacetate;
2-amino-8-[(3′-methoxybiphenyl-3-yl)methyl]-3,8-dimethyl-5,6,7,8-tetrahydroquinazolin-4(3H)-one trifluoroacetate;
2-amino-8-(3-bromobenzyl)-3,8-dimethyl-5,6,7,8-tetrahydroquinazolin-4(3H)-one;
2-amino-8-(3-bromobenzyl)-8-methyl-5,6,7,8-tetrahydroquinazolin-4(3H)-one;
2-amino-3-methyl-5-(2-phenylethyl)pyrimidin-4(3H)-one trifluoroacetate;
2-amino-1-methyl-5-(2-phenylethyl)pyrimidin-4(1H)-one trifluoroacetate;
2-amino-5-(2-phenylethyl)pyrimidin-4(3H)-one trifluoroacetate;
2-amino-5-[2-(3′-methoxybiphenyl-3-yl)ethyl]-3-methylpyrimidin-4(3H)-one trifluoroacetate; and
2-amino-5-[2-(3′-methoxybiphenyl-3-yl)ethyl]pyrimidin-4(3H)-one trifluoroacetate,
or a pharmaceutically acceptable salt, tautomer, or in vivo-hydrolysable precursor thereof.

102. A pharmaceutical composition comprising as active ingredient a therapeutically effective amount of a compound according to claim 1 in association with a pharmaceutically acceptable excipient, carrier or diluent.

103-107. (canceled)

108. A method of inhibiting activity of BACE comprising contacting said BACE with a compound of claim 1.

109. A method of treating or preventing an Aβ-related pathology in a mammal, comprising administering to said patient a therapeutically effective amount of a compound of claim 1.

110. The method of claim 109, wherein said Aβ-related pathology is Downs syndrome, a β-amyloid angiopathy, cerebral amyloid angiopathy, hereditary cerebral hemorrhage, a disorder associated with cognitive impairment, MCI (“mild cognitive impairment”), Alzheimer Disease, memory loss, attention deficit symptoms associated with Alzheimer disease, neurodegeneration associated with Alzheimer disease, dementia of mixed vascular origin, dementia of degenerative origin, pre-senile dementia, senile dementia, dementia associated with Parkinson's disease, progressive supranuclear palsy or cortical basal degeneration.

111. The method of claim 109, wherein said mammal is a human.

112. A method of treating or preventing an Aβ-related pathology in a mammal, comprising administering to said patient a therapeutically effective amount of a compound of claim 1 and at least one cognitive enhancing agent, memory enhancing agent, or choline esterase inhibitor.

113. The method of claim 112, wherein said Aβ-related pathology is Downs syndrome, a β-amyloid angiopathy, cerebral amyloid angiopathy, hereditary cerebral hemorrhage, a disorder associated with cognitive impairment, MCI (“mild cognitive impairment”), Alzheimer Disease, memory loss, attention deficit symptoms associated with Alzheimer disease, neurodegeneration associated with Alzheimer disease, dementia of mixed vascular origin, dementia of degenerative origin, pre-senile dementia, senile dementia, dementia associated with Parkinson's disease, progressive supranuclear palsy or cortical basal degeneration.

114. The method of claim 112, wherein said mammal is a human.
